(19) United States
(12) Patent Application Publication
(10) Pub. No.: US 2022/0228153 A1
(43) Pub. Date: Jul. 21, 2022

(54) COMPOSITIONS AND METHODS FOR CD33 MODIFICATION

(71) Applicant: VOR BIOPHARMA INC., Cambridge, MA (US)

(72) Inventors: John Lydeard, Cambridge, MA (US); Bibhu Prasad Mishra, Wakefield, MA (US)

(73) Assignee: VOR BIOPHARMA INC., Cambridge, MA (US)

(21) Appl. No.: 17/613,387

(22) PCT Filed: May 22, 2020
(86) PCT No.: PCT/US2020/034391

§ 371 Date: Nov. 22, 2021

Related U.S. Application Data
(60) Provisional application No. 62/962,127, filed on Jan. 16, 2020.
(60) Provisional application No. 62/852,238, filed on May 23, 2019.

(51) Int. Cl. C12N 15/113; C12N 5/0789; C12N 9/22

(52) U.S. Cl. CPC C12N 15/1138; C12N 5/0647; C12N 2510/00; C12N 2310/20; C12N 2310/315; C12N 9/22

(57) ABSTRACT

Some aspects of this disclosure provides, e.g., novel cells having a modification (e.g., insertion or deletion) in the endogenous CD33 gene. Some aspects of the disclosure provide compositions, e.g., gRNAs, that can be used to make such a modification.

### RELATED APPLICATIONS

This application claims priority to U.S. Ser. No. 62/852,238 filed May 23, 2019 and U.S. Ser. No. 62/962,127 filed Jan. 16, 2020, the entire contents of each of which are incorporated herein by reference.

### SEQUENCE LISTING

The instant application contains a Sequence Listing which has been submitted electronically in ASCII format and is hereby incorporated by reference in its entirety. Said ASCII copy, created on May 21, 2020, is named V0291.70004WO00—Sequence Listing.txt and is 23,508 bytes in size.

### BACKGROUND

When a cancer patient is administered an anti-CD33 cancer therapy, the therapy can deplete not only CD33+ cancer cells, but also noncancerous CD33+ cells in an “on-target, off-leukemia” effect. Since hematopoietic stem cells (HSCs) and hematopoietic progenitor cells (HPCs) typically express CD33, the loss of the noncancerous CD33+ cells can deplete the hematopoietic system of the patient. To address this depletion, the subject can be administered rescue cells (e.g., HSCs and/or HPCs) comprising a modification in the CD33 gene. These CD33-modified cells can be resistant to the anti-CD33 cancer therapy, and can therefore repopulate the hematopoietic system during or after anti-CD33 therapy.

### SUMMARY OF THE INVENTION

This disclosure provides, e.g., novel cells having a modification (e.g., insertion or deletion) in the endogenous CD33 gene. The disclosure also provides compositions, e.g., gRNAs, that can be used to make such a modification.

### Enumerated Embodiments

1. A gRNA comprising a targeting domain which binds a target domain of Table 1 (e.g., a target domain of any of SEQ ID NOS: 1-8).
2. A gRNA comprising a targeting domain which binds a target domain of any of SEQ ID NOS: 2-4 or SEQ ID NOS: 6-8.
3. A gRNA comprising a targeting domain which binds a target domain of SEQ ID NO: 1.
4. A gRNA comprising a targeting domain which binds a target domain of SEQ ID NO: 5, wherein the targeting domain does not comprise SEQ ID NO: 1.
5. A gRNA comprising a targeting domain which binds a target domain SEQ ID NO: 5, wherein the targeting domain is at least 21 nucleotides in length.
6. The gRNA of any of the preceding embodiments, wherein the targeting domain base pairs or is complementary with at least 10, 11, 12, 13, 14, 15, 16, 17, 18, 19, or 20 nucleotides of the target domain, or wherein the targeting domain comprises 0, 1, 2, or 3 mismatches with the target domain.
7. The gRNA of any of the preceding embodiments, wherein the targeting domain comprises at least 16 (e.g., 17, 18, 19, 20, 21, 22, 23, 24, 25, or 26) consecutive nucleotides of SEQ ID NO: 13.
8. The gRNA of any of the preceding embodiments, wherein the targeting domain comprises at least 16 (e.g., 17, 18, 19, 20, 21, 22, 23, 24, 25, or 26) consecutive nucleotides of SEQ ID NO: 13, and base pairs or is complementary with at least 10, 11, 12, 13, 14, 15, 16, 17, 18, 19, or 20 nucleotides of the target domain.
9. The gRNA of any of the preceding embodiments, wherein said targeting domain is configured to provide a cleavage event (e.g., a single strand break or double strand break) within the target domain, e.g., immediately after nucleotide position 10, 11, 12, 13, 14, 15, 16, 17, 18, 19, or 20 of the target domain.
10. A gRNA comprising a targeting domain that comprises a sequence of any of SEQ ID NOS: 1-4.
11. A gRNA comprising a targeting domain, wherein the targeting domain comprises a sequence of SEQ ID NO: 9.
12. A gRNA comprising a targeting domain, wherein the targeting domain comprises a sequence of SEQ ID NO: 10.
13. A gRNA comprising a targeting domain, wherein the targeting domain comprises a sequence of SEQ ID NO: 11.
14. A gRNA comprising a targeting domain, wherein the targeting domain comprises a sequence of SEQ ID NO: 12.
15. The gRNA of any of the preceding embodiments, which is a single guide RNA (sgRNA).
16. The gRNA of any of the preceding embodiments, wherein the targeting domain is 16 nucleotides or more in length.
17. The gRNA of any of the preceding embodiments, wherein the targeting domain is 16, 17, 18, 19, 20, 21, 22, 23, 24, 25, or 26 nucleotides in length.
18. The gRNA of any of the preceding embodiments, wherein the targeting domain comprises a sequence of any of SEQ ID NOS: 1-4 or 9-12, or the reverse complement thereof, or a sequence having at least 90% or 95% identity to any of the foregoing, or a sequence having no more than 1, 2, or 3 mutations relative to any of the foregoing.
19. The gRNA of embodiment 18, wherein the 2 mutations are not adjacent to each other.
20. The gRNA of embodiment 18, wherein none of the 3 mutations are adjacent to each other.
21. The gRNA of any of embodiments 18-20, wherein the 1, 2, or 3 mutations are substitutions.
22. The gRNA of any of embodiments 18-20, wherein one or more of the mutations is an insertion or deletion.
23. The gRNA of any of the preceding embodiments, wherein the targeting domain comprises a sequence of any of SEQ ID NOS: 1-4.
24. The gRNA of any of the preceding embodiments, wherein the targeting domain comprises a sequence of SEQ ID NO: 1.
25. The gRNA of any of the preceding embodiments, wherein the targeting domain comprises a sequence of SEQ ID NO: 2.
26. The gRNA of any of the preceding embodiments, wherein the targeting domain comprises a sequence of SEQ ID NO: 3.
27 The gRNA of any of the preceding embodiments, wherein the targeting domain comprises a sequence of SEQ ID NO: 4.
28. The gRNA of any of the preceding embodiments, wherein the targeting domain comprises a sequence of SEQ ID NO: 9.
29. The gRNA of any of the preceding embodiments, wherein the targeting domain comprises a sequence of SEQ ID NO: 10.
30. The gRNA of any of the preceding embodiments, wherein the targeting domain comprises a sequence of SEQ ID NO: 11.
31. The gRNA of any of the preceding embodiments, wherein the targeting domain comprises a sequence of SEQ ID NO: 12.
32. The gRNA of any of the preceding embodiments, which comprises one or more chemical modifications (e.g., a chemical modification to a nucleobase, sugar, or backbone portion).
33. The gRNA of any of the preceding embodiments, which comprises one or more 2′O-methyl nucleotide, e.g., at a position described herein.
34. The gRNA of any of the preceding embodiments, which comprises one or more phosphorothioate or thioPACE linkage, e.g., at a position described herein.
35. The gRNA of any of the preceding embodiments, which binds a Cas9 molecule.
36. The gRNA of any one of the preceding embodiments, wherein the targeting domain is about 18-23, e.g., 20 nucleotides in length.
37. The gRNA of any of the preceding embodiments, which binds to a tracrRNA.
38. The gRNA of any of embodiments 1-36, which comprises a scaffold sequence.
39. The gRNA of any of the preceding embodiments, which comprises one or more of (e.g., all of):
a first complementarity domain;
a linking domain;
a second complementarity domain which is complementary to the first complementarity domain;
a proximal domain; and
a tail domain.
   40. The gRNA of any of the preceding embodiments, which comprises a first complementarity domain.
   41. The gRNA of any of the preceding embodiments, which comprises a linking domain.
   42. The gRNA of embodiment 40 or 41, which comprises a second complementarity domain which is complementary to the first complementarity domain.
   43. The gRNA of any of the preceding embodiments, which comprises a proximal domain.
   44. The gRNA of any of the preceding embodiments, which comprises a tail domain.
   45. The gRNA of any of embodiments 39-44, wherein the targeting domain is heterologous to one or more of (e.g., all of):
the first complementarity domain;
the linking domain;
the second complementarity domain which is complementary to the first complementarity domain;
the proximal domain; and
the tail domain.
   46. A kit or composition comprising:
a) a gRNA of any of embodiments 1-45, or a nucleic acid encoding the gRNA, and
b) a second gRNA, or a nucleic acid encoding the second gRNA.
   47. The kit or composition of embodiment 46, wherein the first gRNA comprises a targeting domain that comprises a sequence of CCCCAGGACTACTCACTCCT (SEQ ID NO: 1).
   48. The kit or composition of embodiment 46 or 47, wherein the second gRNA targets a lineage-specific cell-surface antigen.
   49. The kit or composition of any of embodiments 46-48 wherein the second gRNA targets a lineage-specific cell-surface antigen other than CD33.
   50. The kit or composition of any of embodiments 46-49, wherein the second gRNA targets CLL-1 (e.g., wherein the second gRNA comprises a targeting domain that comprises a sequence of GGTGGCTATTGTTTGCAGTG (SEQ ID NO: 23)).
   51. The kit or composition of any of embodiments 46-50, wherein the second gRNA targets CD123 (e.g., wherein the second gRNA comprises a targeting domain that comprises a sequence of TTTCTTGAGCTGCAGCTGGG (SEQ ID NO: 24) or AGTTCCCACATCCTGGTGCG (SEQ ID NO: 25)).
   52. The kit or composition of any of embodiments 46-51, wherein the second gRNA comprises a targeting domain that comprises a sequence of TTTCTTGAGCTGCAGCTGGG (SEQ ID NO: 24).
   53. The kit or composition of any of embodiments 46-52, wherein the second gRNA comprises a targeting domain that comprises a sequence of AGTTCCCACATCCTGGTGCG (SEQ ID NO: 25).
   54. The kit or composition of any of embodiments 46-53, wherein the second gRNA comprises a targeting domain that comprises a sequence of Table A.
   55. The kit or composition of any of embodiments 46-54, wherein the gRNA of (a) comprises a targeting domain that comprises a sequence of CCCCAGGACTACTCACTCCT (SEQ ID NO: 1) and the second gRNA comprises a targeting domain that comprises a sequence of GGTGGCTATTGTTTGCAGTG (SEQ ID NO: 23).
   56. The kit or composition of any of embodiments 46-54, wherein the gRNA of (a) comprises a targeting domain that comprises a sequence of CCCCAGGACTACTCACTCCT (SEQ ID NO: 1) and the second gRNA comprises a targeting domain that comprises a sequence of TTTCTTGAGCTGCAGCTGGG (SEQ ID NO: 24).
   57. The kit or composition of any of embodiments 46-54, wherein the gRNA of (a) comprises a targeting domain that comprises a sequence of CCCCAGGACTACTCACTCCT (SEQ ID NO: 1) and the second gRNA comprises a targeting domain that comprises a sequence of AGTTCCCACATCCTGGTGCG (SEQ ID NO: 25).
   58. The kit or composition of any of embodiments 46-57, which further comprises a third gRNA, or a nucleic acid encoding the third gRNA.
   59. The kit or composition of embodiment 58, wherein the third gRNA targets a lineage-specific cell-surface antigen.
   60. The kit or composition of embodiment 58, wherein the third gRNA targets CD33, CLL-1, or CD123.
   61. The kit or composition of any of embodiments 58-53, wherein the gRNA of (a) comprises a targeting domain that comprises a sequence of CCCCAGGACTACTCACTCCT (SEQ ID NO: 1), the second gRNA comprises a targeting domain that comprises a sequence of TTTCTTGAGCTGCAGCTGGG (SEQ ID NO: 24), and the third gRNA comprises a targeting domain that comprises a sequence of AGTTCCCACATCCTGGTGCG (SEQ ID NO: 25).
   62. The kit or composition of any of embodiments 58-60, wherein the gRNA of (a) comprises a targeting domain that comprises a sequence of CCCCAGGACTACTCACTCCT (SEQ ID NO: 1), the second gRNA comprises a targeting domain that comprises a sequence of TTTCTTGAGCTGCAGCTGGG (SEQ ID NO: 24), and the third gRNA comprises a targeting domain that comprises a sequence of GGTGGCTATTGTTTGCAGTG (SEQ ID NO: 23).
   63. The kit or composition of any of embodiments 58-60, wherein the gRNA of (a) comprises a targeting domain that comprises a sequence of CCCCAGGACTACTCACTCCT (SEQ ID NO: 1), the second gRNA comprises a targeting domain that comprises a sequence of AGTTCCCACATCCTGGTGCG (SEQ ID NO: 25), and the third gRNA comprises a targeting domain that comprises a sequence of GGTGGCTATTGTTTGCAGTG (SEQ ID NO: 23).
   64. The kit or composition of any of embodiments 58-63, which further comprises a fourth gRNA, or a nucleic acid encoding the fourth gRNA.
   65. The kit or composition of embodiment 64, wherein the fourth gRNA targets a lineage-specific cell-surface antigen.
   66. The kit or composition of embodiment 64, wherein the fourth gRNA targets CD33, CLL-1, or CD123.
   67. The kit or composition of any of embodiments 64-66, wherein the gRNA of (a) comprises a targeting domain that comprises a sequence of CCCCAGGACTACTCACTCCT (SEQ ID NO: 1), the second gRNA comprises a targeting domain that comprises a sequence of TTTCTTGAGCTGCAGCTGGG (SEQ ID NO: 24), the third gRNA comprises a targeting domain that comprises a sequence of AGTTCCCACATCCTGGTGCG (SEQ ID NO: 25), and the fourth gRNA comprises a targeting domain that comprises a sequence of GGTGGCTATTGTTTGCAGTG (SEQ ID NO: 23).
   68. The kit or composition of any of embodiments 64-67, wherein the gRNA of (a), the second gRNA, the third gRNA, and the fourth gRNA are admixed.
   69. The kit or composition of any of embodiments 64-67, wherein the gRNA of (a), the second gRNA, the third gRNA, and the fourth gRNA are in separate containers.
   70. The kit or composition of any of embodiments 46-67, wherein (a) and (b) are admixed.
   71. The kit or composition of any of embodiments 46-67, wherein (a) and (b) are in separate containers.
   72. The kit or composition of any of embodiments 46-71, wherein the nucleic acid of (a) and the nucleic acid of (b) are part of the same nucleic acid.
   73. The kit or composition of any of embodiments 46-71, wherein the nucleic acid of (a) and the nucleic acid of (b) are separate nucleic acids.
   74. A genetically engineered hematopoietic cell (e.g., hematopoietic stem or progenitor cell), which comprises:
(a) a mutation at a target domain of Table 1 (e.g., a target domain of any of SEQ ID NOS: 1-8); and
(b) a second mutation at a gene encoding a lineage-specific cell surface antigen other than CD33.
   75. The genetically engineered hematopoietic cell of embodiment 74, wherein the mutation of (a) is at a target domain of SEQ ID NO: 1.
   76. The genetically engineered hematopoietic cell of embodiment 74, wherein the mutation of (a) is at a target domain of SEQ ID NO: 2.
   77. The genetically engineered hematopoietic cell of embodiment 74, wherein the mutation of (a) is at a target domain of SEQ ID NO: 3.
   78. The genetically engineered hematopoietic cell of embodiment 74, wherein the mutation of (a) is at a target domain of SEQ ID NO: 4.
   79. The genetically engineered hematopoietic cell of any of embodiments 74-78, wherein the mutation of (a) comprises an insertion, a deletion, or a substitution (e.g., a single nucleotide variant).
   80. The genetically engineered hematopoietic cell of any of embodiments 74-79, which comprises an insertion of 1 nt or 2 nt, or a deletion of 1 nt, 2 nt, 4 nt, or 5 nt in CD33.
   81. The genetically engineered hematopoietic cell of any of embodiments 74-79, which comprises an indel as described herein, e.g., an indel produced by or producible by a gRNA described herein (e.g., any of gRNA A, gRNA B, gRNA C, or gRNA D).
   82. The genetically engineered hematopoietic cell of any of embodiments 74-79, which comprises an indel produced by or producible by a CRISPR system described herein, e.g., a method of Example 1, 2, 4, 5, or 6.
   83. The genetically engineered cell of embodiment 79, wherein the deletion is fully within the target domain of any of SEQ ID NOS: 1-8.
   84. The genetically engineered cell of embodiment 83, wherein the deletion is 1, 2, 4, 5, 7, 8, 10, 11, 13, 14, 16, or 17 nucleotides in length.
   85. The genetically engineered cell of embodiment 79, wherein the deletion has one or both endpoints outside of the target domain of any of SEQ ID NOS: 1-8.
   86. The genetically engineered cell of any of embodiments 79-85, wherein the mutation results in a frameshift.
   87. The genetically engineered hematopoietic cell of any of embodiments 79-85, wherein the second mutation comprises an insertion, a deletion, or a substitution (e.g., a single nucleotide variant).
   88. Use of a gRNA of any of embodiments 1-45 or a composition or kit of any of embodiments 39-66 for reducing expression of CD33 in a sample of hematopoietic cells stem or progenitor cells using a CRISPR/Cas9 system.
   89. Use of a CRISPR/Cas9 system for reducing expression of CD33 in a sample of hematopoietic cells stem or progenitor cells, wherein the gRNA of the CRISPR/Cas9 system is a gRNA of any of embodiments 1-45, or gRNAs of a composition or kit of any of embodiments 46-73.
   90. A method of producing a genetically engineered cell, comprising:
(i) providing a cell (e.g., a hematopoietic stem or progenitor cell, e.g., a wild-type hematopoietic stem or progenitor cell), and
(ii) introducing into the cell (a) a guide RNA (gRNA) of any of embodiments 1-45 or gRNAs of a composition or kit of any of embodiments 46-73; and (b) an endonuclease that binds the gRNA (e.g., a Cas9 molecule), thereby producing the genetically engineered cell.
   91. A method of producing a genetically engineered cell, comprising:
(i) providing a cell (e.g., a hematopoietic stem or progenitor cell, e.g., a wild-type hematopoietic stem or progenitor cell), and
(ii) introducing into the cell (a) a gRNA of any of embodiments 1-45 or gRNAs of a composition or kit of any of embodiments 46-73; and (b) a Cas9 molecule that binds the gRNA,
thereby producing the genetically engineered cell.
   92. The method or use of any of embodiments 88-91, which results in the genetically engineered hematopoietic stem or progenitor cell having a reduced expression level of CD33 as compared with a wild-type counterpart cell.
   93. The method or use of any of embodiments 88-92, which results in the genetically engineered hematopoietic stem or progenitor cell having a reduced expression level of CD33 that is less than 20% of the level of CD33 in a wild-type counterpart cell.
   94. The method or use of any of embodiments 88-93, which is performed on a plurality of hematopoietic stem or progenitor cells.
   95. The method or use of any of embodiments 88-94, which is performed on a cell population comprising a plurality of hematopoietic stem cells and a plurality of hematopoietic progenitor cells.
   96. The method or use of any of embodiments 88-95, which produces a cell population according to any of embodiments 182-206.
   97. The method of any of embodiment 88-96, wherein the nucleic acids of (a) and (b) are encoded on one vector, which is introduced into the cell.
   98. The method of embodiment 97, wherein the vector is a viral vector.
   99. The method of embodiment 98, wherein (a) and (b) are introduced into the cell as a pre-formed ribonucleoprotein complex.
   100. The method of embodiment 99, wherein the ribonucleoprotein complex is introduced into the cell via electroporation.
   101. The method of any of embodiments 88-100, wherein the endonuclease (e.g., a Cas9 molecule) is introduced into the cell by delivering into the cell a nucleic acid molecule (e.g., an mRNA molecule or a viral vector, e.g., AAV) encoding the endonuclease.
   102. The method of any of embodiments 88-101, wherein the cell (e.g., the hematopoietic stem or progenitor cell) is CD34+.
   103. The method of any of embodiments 88-102, wherein the hematopoietic stem or progenitor cell is from bone marrow cells or peripheral blood mononuclear cells (PBMCs) of a subject.
   104. The method of any of embodiments 88-103, wherein the subject has a hematopoietic disorder, e.g., a hematopoietic malignancy, e.g., a leukemia, e.g., AML.
   105. The method of any of embodiments 88-104, wherein the subject has a cancer, wherein cells of the cancer express CD33 (e.g., wherein at least a plurality of the cancer cells express CD33).
   106. The method or use of any of embodiments 88-105, which results in a mutation that causes a reduced expression level of CD33 as compared with a wild-type counterpart cell.
   107. The method or use of any of embodiments 88-106, which results in a mutation that causes a reduced expression level of wild-type CD33 as compared with a wild-type counterpart cell.
   108. The method or use of any of embodiments 88-107, which produces a genetically engineered hematopoietic stem or progenitor cell having a reduced expression level of CD33 as compared with a wild-type counterpart cell.
   109. The method or use of any of embodiments 88-108, which produces a genetically engineered hematopoietic stem or progenitor cell having a reduced expression level of wild-type CD33 as compared with a wild-type counterpart cell.
   110. A genetically engineered hematopoietic stem or progenitor cell, which is produced by a method of any of embodiments 88-109.
   111. A nucleic acid (e.g., DNA) encoding the gRNA of any of embodiments 1-45.
   112. A genetically engineered cell (e.g., a hematopoietic stem or progenitor cell), which comprises a mutation at a target domain of Table 1 (e.g., a target domain of any of SEQ ID NOS: 1-8), e.g., wherein the mutation is a result of the genetic engineering.
   113. A genetically engineered cell (e.g., a hematopoietic stem or progenitor cell), which comprises a mutation within 100, 90, 80, 70, 60, 50, 40, 30, 20, or 10 nucleotides (upstream or downstream) of a target domain of Table 1 (e.g., a target domain of any of SEQ ID NOS: 1-8).
   114. The genetically engineered cell of embodiment 113, wherein the mutation is within 100, 90, 80, 70, 60, 50, 40, 30, 20, or 10 nucleotides (upstream or downstream) of any of SEQ ID NOS: 1, 2, or 4.
   115. The genetically engineered cell of embodiment 113, wherein the mutation is within 100, 90, 80, 70, 60, 50, 40, 30, 20, or 10 nucleotides downstream of SEQ ID NO: 3.
   116. The genetically engineered cell of embodiment 113, wherein the mutation is within 60, 50, 40, 30, 20, or 10 nucleotides upstream of SEQ ID NO: 3.
   117. A genetically engineered hematopoietic stem or progenitor cell, which comprises a mutation at a target domain of SEQ ID NO: 1.
   118. A genetically engineered hematopoietic stem or progenitor cell, which comprises a mutation at a target domain of SEQ ID NO: 1, wherein the mutation results in a reduced expression level of CD33 as compared with a wild-type counterpart cell.
   119. A genetically engineered hematopoietic stem or progenitor cell, which comprises a mutation at a target domain of SEQ ID NO: 1, wherein the mutation results in a reduced expression level of CD33 that is less than 20% of the level of CD33 in a wild-type counterpart cell.
   120. A genetically engineered hematopoietic stem or progenitor cell, which comprises a mutation at a target domain of SEQ ID NO: 2.
   121. A genetically engineered hematopoietic stem or progenitor cell, which comprises a mutation at a target domain of SEQ ID NO: 2, wherein the mutation results in a reduced expression level of CD33 as compared with a wild-type counterpart cell.
   122. A genetically engineered hematopoietic stem or progenitor cell, which comprises a mutation at a target domain of SEQ ID NO: 2, wherein the mutation results in a reduced expression level of CD33 that is less than 20% of the level of CD33 in a wild-type counterpart cell.
   123. A genetically engineered hematopoietic stem or progenitor cell, which comprises a mutation at a target domain of SEQ ID NO: 3.
   124. A genetically engineered hematopoietic stem or progenitor cell, which comprises a mutation at a target domain of SEQ ID NO: 3, wherein the mutation results in a reduced expression level of CD33 as compared with a wild-type counterpart cell.
   125. A genetically engineered hematopoietic stem or progenitor cell, which comprises a mutation at a target domain of SEQ ID NO: 3, wherein the mutation results in a reduced expression level of CD33 that is less than 20% of the level of CD33 in a wild-type counterpart cell.
   126. A genetically engineered hematopoietic stem or progenitor cell, which comprises a mutation at a target domain of SEQ ID NO: 4.
   127. A genetically engineered hematopoietic stem or progenitor cell, which comprises a mutation at a target domain of SEQ ID NO: 4, wherein the mutation results in a reduced expression level of CD33 as compared with a wild-type counterpart cell.
   128. A genetically engineered hematopoietic stem or progenitor cell, which comprises a mutation at a target domain of SEQ ID NO: 4, wherein the mutation results in a reduced expression level of CD33 that is less than 20% of the level of CD33 in a wild-type counterpart cell.
   129. A genetically engineered hematopoietic stem or progenitor cell, which comprises a mutation at a target domain of SEQ ID NO: 5.
   130. A genetically engineered hematopoietic stem or progenitor cell, which comprises a mutation at a target domain of SEQ ID NO: 5, wherein the mutation results in a reduced expression level of CD33 as compared with a wild-type counterpart cell.
   131. A genetically engineered hematopoietic stem or progenitor cell, which comprises a mutation at a target domain of SEQ ID NO: 5, wherein the mutation results in a reduced expression level of CD33 that is less than 20% of the level of CD33 in a wild-type counterpart cell.
   132. A genetically engineered hematopoietic stem or progenitor cell, which comprises a mutation at a target domain of SEQ ID NO: 6.
   133. A genetically engineered hematopoietic stem or progenitor cell, which comprises a mutation at a target domain of SEQ ID NO: 6, wherein the mutation results in a reduced expression level of CD33 as compared with a wild-type counterpart cell.
   134. A genetically engineered hematopoietic stem or progenitor cell, which comprises a mutation at a target domain of SEQ ID NO: 6, wherein the mutation results in a reduced expression level of CD33 that is less than 20% of the level of CD33 in a wild-type counterpart cell.
   135. A genetically engineered hematopoietic stem or progenitor cell, which comprises a mutation at a target domain of SEQ ID NO: 7.
   136. A genetically engineered hematopoietic stem or progenitor cell, which comprises a mutation at a target domain of SEQ ID NO: 7, wherein the mutation results in a reduced expression level of CD33 as compared with a wild-type counterpart cell.
   137. A genetically engineered hematopoietic stem or progenitor cell, which comprises a mutation at a target domain of SEQ ID NO: 7, wherein the mutation results in a reduced expression level of CD33 that is less than 20% of the level of CD33 in a wild-type counterpart cell.
   138. A genetically engineered hematopoietic stem or progenitor cell, which comprises a mutation at a target domain of SEQ ID NO: 8.
   139. A genetically engineered hematopoietic stem or progenitor cell, which comprises a mutation at a target domain of SEQ ID NO: 8, wherein the mutation results in a reduced expression level of CD33 as compared with a wild-type counterpart cell.
   140. A genetically engineered hematopoietic stem or progenitor cell, which comprises a mutation at a target domain of SEQ ID NO: 8, wherein the mutation results in a reduced expression level of CD33 that is less than 20% of the level of CD33 in a wild-type counterpart cell.
   141. The genetically engineered cell of any of embodiments 112-140, comprising a predicted off target site which does not comprise a mutation or sequence change relative to the sequence of the site prior to gene editing of CD33.
   142. The genetically engineered cell of any of embodiments 112-141, comprising two predicted off target sites which do not comprise a mutation or sequence change relative to the sequence of the site prior to gene editing of CD33.
   143. The genetically engineered cell of any of embodiments 112-142, comprising at least 3, 4, 5, 6, 7, 8, 9, 10, 11, 12, 13, 14, 15, 16, 17, 18, 19, or 20 predicted off target sites which do not comprise a mutation or sequence change relative to the sequence of the site prior to gene editing of CD33.
   144. The genetically engineered cell of any of embodiments 112-143, which does not comprise a mutation in any predicted off-target site, e.g., in any site in the human genome having 1, 2, 3, or 4 mismatches relative to the target domain.
   145. The genetically engineered cell of any of any of embodiments 112-144, which does not comprise a mutation in any site in the human genome having 1 mismatch relative to the target domain.
   146. The genetically engineered cell of any of embodiments 112-145, which does not comprise a mutation in any site in the human genome having 1 or 2 mismatches relative to the target domain.
   147. The genetically engineered cell of any of embodiments 112-146, which does not comprise a mutation in any site in the human genome having 1, 2, or 3 mismatches relative to the target domain.
   148. The genetically engineered cell of any of embodiments 112-147, which does not comprise a mutation in any site in the human genome having 1, 2, 3, or 4 mismatches relative to the target domain.
   149. The genetically engineered cell of any of embodiments any of embodiments 112-148, wherein the mutation comprises an insertion, a deletion, or a substitution (e.g., a single nucleotide variant).
   150. The genetically engineered cell of embodiment 149, wherein the deletion is fully within the target domain of any of SEQ ID NOS: 1-8.
   151. The genetically engineered cell of embodiment 150, wherein the deletion is 1, 2, 4, 5, 7, 8, 10, 11, 13, 14, 16, or 17 nucleotides in length.
   152. The genetically engineered cell of embodiment 149, wherein the deletion has one or both endpoints outside of the target domain of any of SEQ ID NOS: 1-8.
   153. The genetically engineered cell of any of embodiments 112-152, wherein the mutation results in a frameshift.
   154. The genetically engineered cell (e.g., hematopoietic stem or progenitor cell) of any of embodiments 112-153, wherein the mutation results in a reduced expression level of wild-type CD33 as compared with a wild-type counterpart cell (e.g., less than 50%, 40%, 30%, 20%, 15%, 10%, or 5% of the level in the wild-type counterpart cell).
   155. The genetically engineered cell of any of embodiments 112-153, wherein the cell has a reduced level of wild-type CD33 protein as compared with a wild-type counterpart cell (e.g., less than 50%, 40%, 30%, 20%, 15%, 10%, or 5% of the level in the wild-type counterpart cell).
   156. The genetically engineered cell of any of embodiments 112-155, which does not express CD33.
   157. The genetically engineered cell (e.g., hematopoietic stem or progenitor cell) of any of embodiments 112-156, wherein the mutation results in a lack of expression of CD33.
   158. The genetically engineered cell (e.g., hematopoietic stem or progenitor cell) of any of embodiments 112-157, which expresses less than 20% of the CD33 expressed by a wild-type counterpart cell.
   159. The genetically engineered cell (e.g., hematopoietic stem or progenitor cell) of any of embodiments 112-158, wherein the reduced expression level of CD33 is in a cell differentiated from (e.g., terminally differentiated from) the hematopoietic stem or progenitor cell, and the wild-type counterpart cell is a cell differentiated from (e.g., terminally differentiated from) a wild-type hematopoietic stem or progenitor cell.
   160. The genetically engineered cell (e.g., hematopoietic stem or progenitor cell) of embodiment 158, wherein the cell differentiated from the hematopoietic stem or progenitor cell is a myeloblast, monoblast, monocyte, macrophage, or natural killer cell.
   161. The genetically engineered cell of any of embodiments 112-160, which is CD34+.
   162. The genetically engineered cell of any of embodiments 112-161, which is from bone marrow cells or peripheral blood mononuclear cells of a subject.
   163. The genetically engineered cell of embodiment 162, wherein the subject is a human patient having a hematopoietic malignancy, e.g., AML.
   164. The genetically engineered cell of embodiment 162 or 163, wherein the subject has a cancer, wherein cells of the cancer express CD33 (e.g., wherein at least a plurality of the cancer cells express CD33).
   165. The genetically engineered cell of embodiment 162, wherein the subject is a healthy human donor (e.g., an HLA-matched donor).
   166. The genetically engineered cell of any of embodiments 112-165, which further comprises a nuclease chosen from a CRISPR endonuclease, a zinc finger nuclease (ZFN), a transcription activator-like effector-based nuclease (TALEN), or a meganuclease, or a nucleic acid (e.g., DNA or RNA) encoding the nuclease, wherein optionally the nuclease is specific for CD33.
   167. The genetically engineered cell of any of embodiments 112-165, which further comprises a gRNA (e.g., a single guide RNA) specific for CD33, or a nucleic acid encoding the gRNA.
   168. The genetically engineered cell of embodiment 167, wherein the gRNA is a gRNA described herein, e.g., a gRNA of any of embodiments 1-45.
   169. The genetically engineered cell of any of embodiments 112-168, which was made by a process comprising contacting the cell with a nuclease chosen from a CRISPR endonuclease, a zinc finger nuclease (ZFN), a transcription activator-like effector-based nuclease (TALEN), or a meganuclease (e.g., by contacting the cell with the nuclease or a nucleic acid encoding the nuclease).
   170. The genetically engineered cell of any of embodiments 112-168, which was made by a process comprising contacting the cell with a nickase or a catalytically inactive Cas9 molecule (dCas9), e.g., fused to a functional domain e.g., a deaminase or demethylase domain (e.g., by contacting the cell with the nuclease or a nucleic acid encoding the nuclease).
   171. The genetically engineered cell of any of embodiments 112-170, in which both copies of CD33 are mutant.
   172. The genetically engineered cell of embodiment 171, wherein both copies of CD33 have the same mutation.
   173. The genetically engineered cell of embodiment 171, wherein the copies of CD33 have different mutations.
   174. The genetically engineered cell of any of embodiments 112-171, comprising a first copy of CD33 having a first mutation and a second copy of CD33 having a second mutation, wherein the first and second mutations are different.
   175. The genetically engineered cell of embodiment 174, wherein the first copy of CD33 comprises a first deletion.
   176. The genetically engineered cell of embodiment 174 or 175, wherein the second copy of CD33 comprises a second deletion.
   177. The genetically engineered cell of any of embodiments 174-176, wherein the first and second deletions overlap.
   178. The genetically engineered cell of any of embodiments 174-177, wherein an endpoint of the first deletion is within the second deletion.
   179. The genetically engineered cell of any of embodiments 174-178, wherein both endpoints of the first deletion are within the second deletion.
   180. The genetically engineered cell of any of embodiments 174-176, wherein the first and second deletion share an endpoint.
   181. The genetically engineered cell of any of embodiments 174-176, wherein the first and second mutation are each independently selected from: an insertion of 1 nt or 2 nt, or a deletion of 1 nt, 2 nt, 4 nt, or 5 nt.
   182. A cell population, comprising a plurality of the genetically engineered hematopoietic stem or progenitor cells of any embodiments 112-181 (e.g., comprising hematopoietic stem cells, hematopoietic progenitor cells, or a combination thereof).
   183. A cell population, comprising a plurality of genetically engineered hematopoietic stem or progenitor cells which comprise a mutation at a target domain of SEQ ID NO: 1.
   184. A cell population, comprising a plurality of genetically engineered hematopoietic stem or progenitor cells which comprise a mutation at a target domain of SEQ ID NO: 1, wherein the mutation results in a reduced expression level of CD33 as compared with a wild-type counterpart cell population.
   185. A cell population, comprising a plurality of genetically engineered hematopoietic stem or progenitor cells which comprise a mutation at a target domain of SEQ ID NO: 1, wherein the mutation results in a reduced expression level of CD33 that is less than 20% of the level of CD33 in a wild-type counterpart cell population.
   186. A cell population, comprising a plurality of genetically engineered hematopoietic stem or progenitor cells which comprise a mutation at a target domain of SEQ ID NO: 2.
   187. A cell population, comprising a plurality of genetically engineered hematopoietic stem or progenitor cells which comprise a mutation at a target domain of SEQ ID NO: 2, wherein the mutation results in a reduced expression level of CD33 as compared with a wild-type counterpart cell population.
   188. A cell population, comprising a plurality of genetically engineered hematopoietic stem or progenitor cells which comprise a mutation at a target domain of SEQ ID NO: 2, wherein the mutation results in a reduced expression level of CD33 that is less than 20% of the level of CD33 in a wild-type counterpart cell population.
   189. A cell population, comprising a plurality of genetically engineered hematopoietic stem or progenitor cells which comprise a mutation at a target domain of SEQ ID NO: 3.
   190. A cell population, comprising a plurality of genetically engineered hematopoietic stem or progenitor cells which comprise a mutation at a target domain of SEQ ID NO: 3, wherein the mutation results in a reduced expression level of CD33 as compared with a wild-type counterpart cell population.
   191. A cell population, comprising a plurality of genetically engineered hematopoietic stem or progenitor cells which comprise a mutation at a target domain of SEQ ID NO: 3, wherein the mutation results in a reduced expression level of CD33 that is less than 20% of the level of CD33 in a wild-type counterpart cell population.
   192. A cell population, comprising a plurality of genetically engineered hematopoietic stem or progenitor cells which comprise a mutation at a target domain of SEQ ID NO: 4.
   193. A cell population, comprising a plurality of genetically engineered hematopoietic stem or progenitor cells which comprise a mutation at a target domain of SEQ ID NO: 4, wherein the mutation results in a reduced expression level of CD33 as compared with a wild-type counterpart cell population.
   194. A cell population, comprising a plurality of genetically engineered hematopoietic stem or progenitor cells which comprise a mutation at a target domain of SEQ ID NO: 4, wherein the mutation results in a reduced expression level of CD33 that is less than 20% of the level of CD33 in a wild-type counterpart cell population.
   195. A cell population, comprising a plurality of genetically engineered hematopoietic stem or progenitor cells which comprise a mutation at a target domain of SEQ ID NO: 5.
   196. A cell population, comprising a plurality of genetically engineered hematopoietic stem or progenitor cells which comprise a mutation at a target domain of SEQ ID NO: 5, wherein the mutation results in a reduced expression level of CD33 as compared with a wild-type counterpart cell population.
   197. A cell population, comprising a plurality of genetically engineered hematopoietic stem or progenitor cells which comprise a mutation at a target domain of SEQ ID NO: 5, wherein the mutation results in a reduced expression level of CD33 that is less than 20% of the level of CD33 in a wild-type counterpart cell population.
   198. A cell population, comprising a plurality of genetically engineered hematopoietic stem or progenitor cells which comprise a mutation at a target domain of SEQ ID NO: 6.
   199. A cell population, comprising a plurality of genetically engineered hematopoietic stem or progenitor cells which comprise a mutation at a target domain of SEQ ID NO: 6, wherein the mutation results in a reduced expression level of CD33 as compared with a wild-type counterpart cell population.
   200. A cell population, comprising a plurality of genetically engineered hematopoietic stem or progenitor cells which comprise a mutation at a target domain of SEQ ID NO: 6, wherein the mutation results in a reduced expression level of CD33 that is less than 20% of the level of CD33 in a wild-type counterpart cell population.
   201. A cell population, comprising a plurality of genetically engineered hematopoietic stem or progenitor cells which comprise a mutation at a target domain of SEQ ID NO: 7.
   202. A cell population, comprising a plurality of genetically engineered hematopoietic stem or progenitor cells which comprise a mutation at a target domain of SEQ ID NO: 7, wherein the mutation results in a reduced expression level of CD33 as compared with a wild-type counterpart cell population.
   203. A cell population, comprising a plurality of genetically engineered hematopoietic stem or progenitor cells which comprise a mutation at a target domain of SEQ ID NO: 7, wherein the mutation results in a reduced expression level of CD33 that is less than 20% of the level of CD33 in a wild-type counterpart cell population.
   204. A cell population, comprising a plurality of genetically engineered hematopoietic stem or progenitor cells which comprise a mutation at a target domain of SEQ ID NO: 8.
   205. A cell population, comprising a plurality of genetically engineered hematopoietic stem or progenitor cells which comprise a mutation at a target domain of SEQ ID NO: 8, wherein the mutation results in a reduced expression level of CD33 as compared with a wild-type counterpart cell population.
   206. A cell population, comprising a plurality of genetically engineered hematopoietic stem or progenitor cells which comprise a mutation at a target domain of SEQ ID NO: 8, wherein the mutation results in a reduced expression level of CD33 that is less than 20% of the level of CD33 in a wild-type counterpart cell population.
   207. The cell population of any of embodiments 182-206, wherein the cell population can differentiate into a cell type which expresses CD33 at a level that is reduced with regard to the level of CD33 expressed by the same differentiated cell type which is derived from a CD33-wildtype hematopoietic stem or progenitor cell.
   208. The cell population of any of embodiments 182-207, wherein the hematopoietic stem or progenitor cells are engineered such that a myeloid progenitor cell descended therefrom is deficient in CD33 levels as compared with a myeloid progenitor cell descended from a CD33-wildtype hematopoietic stem or progenitor cell.
   209. The cell population of any of embodiments 182-208, wherein the hematopoietic stem or progenitor cells are engineered such that a myeloid cell (e.g., a terminally differentiated myeloid cell) descended therefrom is deficient in CD33 levels as compared with a myeloid cell (e.g., a terminally differentiated myeloid cell) descended from a CD33-wildtype hematopoietic stem or progenitor cell.
   210. The cell population of any of embodiments 182-209, which further comprises one or more cells that comprise one or more non-engineered CD33 genes.
   211. The cell population of any of embodiments 182-210, which further comprises one or more cells that are homozygous wild-type for CD33.
   212. The cell population of any of embodiments 182-211, wherein about 0-1%, 1-2%, 2-5%, 5-10%, 10-15%, or 15-20% of cells in the population are homozygous wild-type for CD33, e.g., are hematopoietic stem or progenitor cells that are homozygous wild-type for CD33.
   213. The cell population of any of embodiments 182-212 which further comprises one or more cells that are heterozygous wild-type for CD33.
   214. The cell population of any of embodiments 182-213, wherein about 0-1%, 1-2%, 2-5%, 5-10%, 10-15%, or 15-20% of cells in the population are heterozygous wild-type for CD33, e.g., are hematopoietic stem or progenitor cells that comprise one wild-type copy of CD33 and one mutant copy of CD33.
   215. The cell population of any of embodiments 182-214, wherein at least 40%, 50%, 60%, 70%, 75%, 80%, 85%, 90%, or 95% of the copies of CD33 in the population are mutant.
   216. The cell population of any of embodiments 182-215, which comprises a plurality of different CD33 mutations, e.g., which comprises at least 2, 3, 4, 5, 6, 7, 8, 9, or 10 different mutations.
   217. The cell population of any of embodiments 182-216, which comprises at least 2, 3, 4, 5, 6, 7, 8, 9, or 10 different mutations.
   218. The cell population of any of embodiments 182-216, which comprises at 2, 3, 4, 5, 6, 7, 8, 9, or 10 different insertions.
   219. The cell population of any of embodiments 182-218, which comprises a plurality of insertions and a plurality of deletions.
   220. The cell population of any of embodiments 182-219, which expresses less than 20% of the CD33 expressed by a wild-type counterpart cell population.
   221. The cell population of any of embodiments 182-220, wherein at least 40%, 50%, 60%, 70%, 75%, 80%, 85%, 90%, or 95% of cells in the population do not express CD33, e.g., as determined using a flow cytometry assay, e.g., wherein cells are contacted with an anti-CD33 antibody (e.g., antibody P67.7), e.g., using an assay according to Example 1.
   222. The cell population of any of embodiments 182-221, wherein the reduced expression level of CD33 is in a cell differentiated from (e.g., terminally differentiated from) the hematopoietic stem or progenitor cell, and the wild-type counterpart cell is a cell differentiated from (e.g., terminally differentiated from) a wild-type hematopoietic stem or progenitor cell.
   223. The cell population of embodiment 222, wherein the cell differentiated from the hematopoietic stem or progenitor cell is a myeloblast, monoblast, monocyte, macrophage, or natural killer cell.
   224. The cell population of any of embodiments 182-223, in which at least 80%, 85%, 90%, or 95% of cells in the population are viable cells.
   225. The cell population of any of embodiments 182-224, wherein one or more of the genetically engineered cells of the population (e.g., at least 10%, 20%, 30%, or 40% of the genetically engineered cells in the population) is a LT-HSC.
   226. The cell population of any of embodiments 182-225, wherein one or more of the genetically engineered cells of the population (e.g., at least 10%, 20%, 30%, or 40% of the genetically engineered cells in the population) is CD38−CD34+CD45RA−CD90+CD49f+, e.g., as determined by flow cytometry, e.g., according to an assay of Example 6.
   227. The cell population of any of embodiments 182-226, which, when administered to a subject, produces CD45+ cells in the subject, e.g., when assayed at 8, 12, or 16 weeks after administration.
   228. The cell population of embodiment 227, which produces levels of hCD45+ cells comparable to the levels of CD45+ cells produced with an otherwise similar cell population that is CD33 wildtype.
   229. The cell population of embodiments 227 or 228, which produces levels of CD45+ cells that is at least 70%, 80%, 85%, 90%, or 95% the levels of hCD45+ cells produced by an otherwise similar cell population that is CD33 wildtype.
   230. The cell population of any of embodiments 182-229, which, when administered to a subject, produces CD14+ cells in the subject, e.g., when assayed at 8, 12, or 16 weeks after administration.
   231. The cell population of embodiment 230, which produces levels of hCD45+ cells comparable to the levels of CD14+ cells produced with an otherwise similar cell population that is CD33 wildtype.
   232. The cell population of embodiments 230 or 231, which produces levels of CD45+ cells that is at least 70%, 80%, 85%, 90%, or 95% the levels of hCD14+ cells produced by an otherwise similar cell population that is CD33 wildtype.
   233. The cell population of any of embodiments 182-232, which, when administered to a subject, produces CD11b+ cells in the subject, e.g., when assayed at 8, 12, or 16 weeks after administration.
   234. The cell population of embodiment 233, which produces levels of hCD45+ cells comparable to the levels of CD11b+ cells produced with an otherwise similar cell population that is CD33 wildtype.
   235. The cell population of embodiments 233 or 235, which produces levels of CD45+ cells that is at least 70%, 80%, 85%, 90%, or 95% the levels of hCD11b+ cells produced by an otherwise similar cell population that is CD33 wildtype.
   236. The cell population of any of embodiments 182-235, which, when administered to a subject, produces CD19+ cells in the subject, e.g., when assayed at 8, 12, or 16 weeks after administration.
   237. The cell population of embodiment 236, which produces levels of hCD45+ cells comparable to the levels of CD19+ cells produced with an otherwise similar cell population that is CD33 wildtype.
   238. The cell population of embodiments 236 or 237, which produces levels of CD45+ cells that is at least 70%, 80%, 85%, 90%, or 95% the levels of hCD19+ cells produced by an otherwise similar cell population that is CD33 wildtype.
   239. The cell population of any of embodiments 182-238, which, when administered to a subject, produces lymphoid cells, monocytes, granulocytes, or neutrophils, or any combination thereof, in the subject, e.g., when assayed at 8, 12, or 16 weeks after administration.
   240. The cell population of embodiment 239, which produces levels of lymphoid cells, monocytes, granulocytes, or neutrophils, or any combination thereof comparable to the levels of said cell type produced with an otherwise similar cell population that is CD33 wildtype.
   241. The cell population of embodiments 239 or 240, which produces levels of lymphoid cells, monocytes, granulocytes, or neutrophils, or any combination thereof that is at least 70%, 80%, 85%, 90%, or 95% the levels of said cell type produced by an otherwise similar cell population that is CD33 wildtype.
   242. The cell population of any of embodiments 227-241, wherein the produced cells are detected in a blood sample, a bone marrow sample, or a spleen sample obtained from the subject.
   243. The cell population of any of embodiments 182-242, which, when administered to a subject, persists for at least 8, 12, or 16 weeks in the subject.
   244. The cell population of any of embodiments 182-243, which, when administered to a subject, provides multilineage hematopoietic reconstitution.
   245. The cell population of any of embodiments 182-244, which, when administered to a subject, produces uncommitted progenitor cells, optionally wherein the level of uncommitted progenitor cells, is comparable to the levels of said cell type produced with an otherwise similar cell population that is CD33 wildtype, optionally wherein the level is at least 70%, 80%, 85%, 90%, or 95% the levels of said cell type produced by an otherwise similar cell population that is CD33 wildtype.
   246. The cell population of any of embodiments 182-244, which, when administered to a subject, produces hCD34+hCD38− cells, optionally wherein the level of uncommitted progenitor cells, is comparable to the levels of said cell type produced with an otherwise similar cell population that is CD33 wildtype, optionally wherein the level is at least 70%, 80%, 85%, 90%, or 95% the levels of said cell type produced by an otherwise similar cell population that is CD33 wildtype.
   247. The cell population of any of embodiments 182-246, which, when administered to a subject, produces committed progenitor cells, optionally wherein the level of uncommitted progenitor cells, is comparable to the levels of said cell type produced with an otherwise similar cell population that is CD33 wildtype, optionally wherein the level is at least 70%, 80%, 85%, 90%, or 95% the levels of said cell type produced by an otherwise similar cell population that is CD33 wildtype.
   248. The cell population of any of embodiments 182-247, which, when administered to a subject, produces hCD34+hCD38+ cells, optionally wherein the level of uncommitted progenitor cells, is comparable to the levels of said cell type produced with an otherwise similar cell population that is CD33 wildtype, optionally wherein the level is at least 70%, 80%, 85%, 90%, or 95% the levels of said cell type produced by an otherwise similar cell population that is CD33 wildtype.
   249. The cell population of any of embodiments 182-248, which, when administered to a subject, produces CD3+ T cells, optionally wherein the level of uncommitted progenitor cells, is comparable to the levels of said cell type produced with an otherwise similar cell population that is CD33 wildtype, optionally wherein the level is at least 70%, 80%, 85%, 90%, or 95% the levels of said cell type produced by an otherwise similar cell population that is CD33 wildtype.
   250. The cell population of any of embodiments 182-249, which, when administered to a subject, produces CD123+ cells, optionally wherein the level of uncommitted progenitor cells, is comparable to the levels of said cell type produced with an otherwise similar cell population that is CD33 wildtype, optionally wherein the level is at least 70%, 80%, 85%, 90%, or 95% the levels of said cell type produced by an otherwise similar cell population that is CD33 wildtype.
   251. The cell population of any of embodiments 182-250, which, when administered to a subject, produces CD10+ cells, optionally wherein the level of uncommitted progenitor cells, is comparable to the levels of said cell type produced with an otherwise similar cell population that is CD33 wildtype, optionally wherein the level is at least 70%, 80%, 85%, 90%, or 95% the levels of said cell type produced by an otherwise similar cell population that is CD33 wildtype.
   252. The cell population of any of embodiments 182-251, which comprises hematopoietic stem cells and hematopoietic progenitor cells.
   253. A pharmaceutical composition comprising the genetically engineered hematopoietic stem or progenitor cell of any of embodiments 112-181.
   254. A pharmaceutical composition comprising the cell population of any of embodiments 182-251.
   255. A mixture, e.g., a reaction mixture, comprising any two or all of:
a) a gRNA of any of embodiments 1-45, or gRNAs of a composition or kit of any of embodiments 46-73;
b) a cell, e.g., a hematopoietic cell, e.g., an HSC or HPC, e.g., a genetically engineered cell of any of embodiments 112-181.
   256. The mixture, e.g., reaction mixture, of embodiment 255, wherein the cell is a wild-type cell or a cell having a mutation in CD33.
   257. A kit comprising any two or more (e.g., three or all) of:
a) a gRNA of any of embodiments 1-45 or gRNAs of a composition or kit of any of embodiments 46-73;
b) a cell, e.g., a hematopoietic cell, e.g., an HSC or HPC, e.g., a genetically engineered cell of any of embodiments 112-181;
c) a Cas9 molecule; and
d) agent that targets CD33, e.g., an agent as described herein.
   258. The kit of embodiment 253, which comprises (a) and (b), (a) and (c), (a) and d), (b) and (c), (b) and (d), or (c) and (d).
   259. A method of making the genetically engineered cell (e.g., hematopoietic stem or progenitor cell) of any of embodiments 112-181, or the cell population of any of embodiments 182-251, which comprises:
(i) providing a cell (e.g., a hematopoietic stem or progenitor cell, e.g., a wild-type hematopoietic stem or progenitor cell), and
(ii) introducing into the cell a nuclease (e.g., an endonuclease) that cleaves the target domain, thereby producing a genetically engineered hematopoietic stem or progenitor cell.
   260. The method of embodiment 259, wherein (ii) comprises introducing into the cell a gRNA that binds the target domain (e.g., a gRNA of any of embodiments 1-45) and an endonuclease that binds the gRNA.
   261. The method of embodiment 259, wherein the endonuclease is a ZFN, TALEN, or meganuclease.
   262. A method of supplying HSCs, HPCs, or HSPCs to a subject, comprising administering to the subject a plurality of cells of embodiment 112-181, or the cell population of any of embodiments 182-251.
   263. A method, comprising administering to a subject in need thereof a plurality of cells of embodiment 112-181, or the cell population of any of embodiments 182-251.
   264. The method of embodiment 262 or 263, wherein the subject has a cancer, wherein cells of the cancer express CD33 (e.g., wherein at least a plurality of the cancer cells express CD33).
   265. The method of any of embodiments 262-264, which further comprises administering to the subject an effective amount of an agent that targets CD33, and wherein the agent comprises an antigen-binding fragment that binds CD33.
   266. The method of embodiment 265, wherein the agent that targets CD33 is an immune cell expressing a chimeric antigen receptor (CAR), which comprises the antigen-binding fragment that binds CD33.
   267. A genetically engineered hematopoietic stem or progenitor cell of any of embodiments 112-181 or a cell population of any of embodiments 182-251 for use in treating a hematopoietic disorder, wherein the treating comprises administering to a subject in need thereof an effective amount of the genetically engineered hematopoietic stem or progenitor cell or the cell population, and further comprises administering to the subject an effective amount of an agent that targets CD33, wherein the agent comprises an antigen-binding fragment that binds CD33.
   268. An agent that targets CD33, wherein the agent comprises an antigen-binding fragment that binds CD33, for use in treating a hematopoietic disorder, wherein the treating comprises administering to a subject in need thereof an effective amount of the agent that targets CD33, and further comprises administering to the subject an effective amount of a genetically engineered hematopoietic stem or progenitor cell of any of embodiments 112-181 or a cell population of any of embodiments 182-251.
   269. A combination of a genetically engineered hematopoietic stem or progenitor cell of any of embodiments 112-181 or a cell population of any of embodiments 182-251, and an agent that targets CD33, wherein the agent comprises an antigen-binding fragment that binds CD33, for use in treating a hematopoietic disorder, wherein the treating comprises administering to a subject in need thereof an effective amount of the genetically engineered hematopoietic stem or progenitor cell or the cell population, and the agent that binds CD33.
   270. A genetically engineered hematopoietic stem or progenitor cell of any of embodiments 112-181 or a cell population of any of embodiments 182-251 for use in cancer immunotherapy.
   275. A genetically engineered hematopoietic stem or progenitor cell of any of embodiments 112-181 or a cell population of any of embodiments 182-251 for use in cancer immunotherapy, wherein the subject has a hematopoietic disorder.
   276. A genetically engineered hematopoietic stem or progenitor cell of any of embodiments 112-181 or a cell population of any of embodiments 182-251 for use in hematopoietic repopulation of a subject having a hematopoietic disorder.
   277. A genetically engineered hematopoietic stem or progenitor cell of any of embodiments 112-181 or a cell population of any of embodiments 182-251 for use in a method of treating a hematopoietic disorder, whereby the genetically engineered hematopoietic stem or progenitor cell described herein or a cell population described herein repopulate the subject.
   278. A genetically engineered hematopoietic stem or progenitor cell of any of embodiments 112-181 or a cell population of any of embodiments 182-251 for use in reducing cytotoxic effects of an agent that targets CD33 in immunotherapy.
   275. A genetically engineered hematopoietic stem or progenitor cell of any of embodiments 112-181 or a cell population of any of embodiments 182-251 for use in an immunotherapy method using an agent that targets CD33, whereby the genetically engineered hematopoietic stem or progenitor cell described herein or a cell population described herein reduces cytotoxic effects of the agent that targets CD33.
   276. The method, cell, agent, or combination of any of embodiments 262-275, wherein the genetically engineered hematopoietic stem or progenitor cell or the cell population is administered concomitantly with the agent that targets CD33.
   277. The method, cell, agent, or combination of any of embodiments 262-275, wherein the genetically engineered hematopoietic stem or progenitor cell or the cell population is administered prior to the agent that targets CD33.
   278. The method, cell, agent, or combination of any of embodiments 262-275, wherein the agent that targets CD33 is administered prior to the genetically engineered hematopoietic stem or progenitor cell or the cell population.
   279. The method, cell, agent, or combination of any of embodiments 262-278, wherein the immune cell is a T cell.
   280. The method, cell, agent, or combination of any of embodiments 262-279, wherein the immune cell, the genetically engineered hematopoietic stem and/or progenitor cell, or both, are allogeneic. 281. The method, cell, agent, or combination of any of embodiments 262-279, wherein the immune cell, the genetically engineered hematopoietic stem and/or progenitor cell, or both, are autologous.
   282. The method, cell, agent, or combination of any of embodiments 262-281, wherein the antigen-binding fragment in the chimeric receptor is a single-chain antibody fragment (scFv) that specifically binds human CD33.
   283. The method, cell, agent, or combination of any of embodiments 262-282, wherein hematopoietic disorder is a cancer, and wherein at least a plurality of cancer cells in the cancer express CD33.
   284. The method, cell, agent, or combination of any of embodiments 262-283, wherein the subject has a hematopoietic malignancy, e.g., a hematopoietic malignancy chosen from Hodgkin's lymphoma, non-Hodgkin's lymphoma, leukemia (e.g., acute myeloid leukemia, acute lymphoid leukemia, chronic myelogenous leukemia, acute lymphoblastic leukemia or chronic lymphoblastic leukemia, and chronic lymphoid leukemia), or multiple myeloma.

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, the Drawings, the Examples, and the Claims.

### BRIEF DESCRIPTION OF THE DRAWINGS

FIG. 1 is a graph showing the gene editing efficiency of different CD33 gRNAs as measured by TIDE analysis. The x axis indicates the gRNA assayed and the y axis indicates the percentage of cells having insertions or deletions at the gRNA target locus. The four bars for each gRNA indicate the four different donors of the HSCs.

FIG. 2 is a graph showing the gene editing efficiency of different CD33 gRNAs as measured by FACS analysis. The x axis indicates the gRNA assayed and the y axis indicates the percentage of cells that are positive for CD33 surface expression. The four bars for each gRNA indicate the four different donors of the HSCs.

FIG. 3 is a graph showing the gene editing efficiency of different CD33 gRNAs as measured by TIDE analysis. The x axis indicates the gRNA assayed and the y axis indicates the percentage of cells having insertions or deletions at the gRNA target locus. The four bars for each gRNA indicate the four different donors of the HSCs.

FIG. 4 is a graph showing the gene editing efficiency of different CD33 gRNAs as measured by FACS analysis. The x axis indicates the gRNA assayed and the y axis indicates the percentage of cells that are positive for CD33 surface expression. The three bars for each gRNA indicate the three different donors of the HSCs.

FIGS. 5A-5D include diagrams showing the results of a TIDE assay showing efficient multiplex genomic editing of both CD19 and CD33. 5A: a chart showing genomic editing of CD19, CD33, and CD19+CD33 in NALM-6 cells. 5B: a chart showing genomic editing of CD19, CD33, and CD19+CD33 in HSCs. 5C: a chart showing genomic editing of CD19, CD33, and CD19+CD33 in HL-60 cells. 5D: a chart showing genomic editing of CD19, CD33, and both CD19 and CD33 in NALM-6 cells.

FIGS. 6A-6C include diagrams showing the results of a nucleofection assay showing the effect of multiplex genomic editing of both CD19 and CD33 on viability in HSCs and cell lines as compared to single RNA nucleofection. The gRNAs used in the nucleofections are indicated on the X-axis. 6A: a chart showing percent viability of HSC cells following genome editing. 6B: a chart showing percent viability of Nalm-6 cells following genome editing. From left to right, each set of three bars corresponds to zero, 24 h, and 48 h. 6C: a chart showing percent viability of HL-60 cells following genome editing. From left to right, each set of four bars corresponds to zero, 48 h, 96 h, and 7d.

FIG. 7 shows target expression on AML cell lines. The expression of CD33, CD123, and CLL1 in MOLM-13 and THP-1 cells and an unstained control was determined by flow cytometric analysis. The X-axis indicates the intensity of antibody staining and the Y-axis corresponds to number of cells.

FIG. 8 shows CD33- and CD123-modified MOLM-13 cells. The expression of CD33 and CD123 in wild-type (WT), CD33−/−, CD123−/− and CD33−/−CD123−/− MOLM-13 cells was assessed by flow cytometry. For the generation of CD33−/− or CD123−/− MOLM-13 cells, WT MOLM-13 cells were electroporated with CD33- or CD123-targeting RNP, followed by flow cytometric sorting of CD33- or CD123-negative cells. CD33−/−CD123−/−MOLM-13 cells were generated by electroporating CD33−/− cells with CD123-targeting RNP and sorted for CD123-negative population. The X-axis indicates the intensity of antibody staining and the Y-axis corresponds to number of cells.

FIG. 9 shows an in vitro cytotoxicity assay of CD33 and CD123 CAR-Ts. Anti-CD33 CAR-T and anti-CD123 CAR-T were incubated with wild-type (WT), CD33^{-/-}, CD123⁻/−, and CD33^{-/-} CD123^{-/-} MOLM-13 cells, and cytotoxicity was assessed by flow cytometry.

Non-transduced T cells were used as mock CAR-T control. The CARpool group was composed of 1:1 pooled combination of anti-CD33 and anti-CD123 CAR-T cells. Student's t test was used. ns=not significant; *P <0.05; **P <0.01. The Y-axis indicates the percentage of specific killing.

FIG. 10 shows CD33- and CLL1-modified HL-60 cells. The expression of CD33 and CLL1 in wild-type (WT), CD33^{-/-}, CLL1^{-/-}, and CD33^{-/-} CLL1^{-/-} HL-60 cells was assessed by flow cytometry. For the generation of CD33^{-/-} or CLL1^{-/-} HL-60 cells, WT HL-60 cells were electroporated with CD33- or CLL1-targeting RNP, followed by flow cytometric sorting of CD33- or CLL1-negative cells. CD33^{-/-} CLL1^{-/-} HL-60 cells were generated by electroporating CD33^{-/-} cells with CLL1-targeting RNP and sorted for CLL1-negative population. The X-axis indicates the intensity of antibody staining and the Y-axis corresponds to number of cells.

FIG. 11 shows an in vitro cytotoxicity assay of CD33 and CLL1 CAR-Ts. Anti-CD33 CAR-T and anti-CLL1 CAR-T were incubated with wild-type (WT), CD33^{-/-}, CLL1^{-/-}, and CD33^{-/-} CLL1^{-/-} HL-60 cells, and cytotoxicity was assessed by flow cytometry. Non-transduced T cells were used as mock CAR-T control. The CARpool group was composed of 1:1 pooled combination of anti-CD33 and anti-CLL-1 CAR-T cells. Student's t test was used. ns=not significant; *P <0.05; **P <0.01, ***P <0.001, ****P <0.0001. The Y-axis indicates the percentage of specific killing.

FIG. 12 shows gene-editing efficiency of CD34+ cells. Human CD34+ cells were electroporated with Cas9 protein and CD33-, CD123-, or CLL1-targeting gRNAs, either alone or in combination. Editing efficiency of CD33, CD123, or CLL1 locus was determined by Sanger sequencing and TIDE analysis. The Y-axis indicates the editing efficiency (% by TIDE).

FIGS. 13A-13C shows in vitro colony formation of gene-edited CD34+ cells. Control or CD33, CD123, CLL-1-modified CD34+ cells were plated in Methocult 2 days after electroporation and scored for colony formation after 14 days. BFU-E: burst forming unit-erythroid; CFU-GM: colony forming unit-granulocyte/macrophage; CFU-GEMM: colony forming unit of multipotential myeloid progenitor cells (generate granulocytes, erythrocytes, monocytes, and megakaryocytes). Student's t test was used.

FIGS. 14A-14C include diagrams and a table showing analysis of populations of CD34+HSCs edited with CD33 gRNA A, at various times following treatment with gemtuzumab ozogamicin (GO). 14A: a photograph showing analysis of CD33 editing following treatment with gemtuzumab ozogamicin. Percentage of edited cells in the sample edited using CD33 gRNA A (“KO”) was assessed by TIDE analysis. 14B: a chart showing the percent CD14+ cells (myeloid differentiation) in the indicated cell populations in the absence of gemtuzumab ozogamicin over time as indicated. 14C: a chart showing the percent CD14+ cells (myeloid differentiation) in the indicated cell populations following treatment with gemtuzumab ozogamicin over time as indicated.

FIG. 15 shows the viability of CD33KO mPB CD34+ HSPCs edited by gRNA A, gRNA B, gRNA O, or gCtrl (control) over the time indicated post-electroporation and editing.

FIG. 16 is a schematic of the flow cytometry analysis and gating protocol used to analyze cells isolated from the blood, spleen, and bone marrow of NSG mice engrafted with CD33KO cells or control cells.

FIGS. 17A-17D shows quantification of hCD33+ cells, hCD45+ cells, hCD14+ cells, or CD11b+ cells per μL of blood, respectively, at week 8 following engraftment in mice with control cells or CD33KO cells edited by the gRNA indicated (gRNAs from left to right on the X-axis: control, O, A or B).

FIGS. 18A-18C shows quantification of the percentage of hCD45+ cells at weeks 8, 12, or 16, respectively, in the blood following engraftment in mice with control cells or CD33KO cells edited by the gRNA indicated (gRNAs from left to right on the X-axis: control, O, A or B).

FIGS. 19A-19C shows quantification of the percentage of hCD33+ cells at weeks 8, 12, or 16, respectively, in the blood following engraftment in mice with control cells or CD33KO cells edited by the gRNA indicated (gRNAs from left to right on the X-axis: control, O, A or B).

FIGS. 20A-20C shows quantification of the percentage of hCD19+ cells at weeks 8, 12, or 16, respectively, in the blood following engraftment in mice with control cells or CD33KO cells edited by the gRNA indicated (gRNAs from left to right on the X-axis: control, O, A or B).

FIGS. 21A-21C shows quantification of the percentage of hCD14+ cells at weeks 8, 12, or 16, respectively, in the blood following engraftment in mice with control cells or CD33KO cells edited by the gRNA indicated (gRNAs from left to right on the X-axis: control, O, A or B).

FIGS. 22A-22C shows quantification of the percentage of hCD11b+ cells at weeks 8, 12, or 16, respectively, in the blood following engraftment in mice with control cells or CD33KO cells edited by the gRNA indicated (gRNAs from left to right on the X-axis: control, O, A or B).

FIGS. 23A-23C shows quantification of the percent of CD33+CD14+(left graphs) or CD33KO derived monocytes (hCD33−CD14+) (right graphs) at weeks 8, 12, or 16, respectively, in the blood following engraftment in mice with control cells or CD33KO cells edited by the gRNA indicated (gRNAs from left to right on the X-axis: control, O, A or B).

FIGS. 24A-24B shows quantification of the percentage of hCD45+ cells or hCD33+ cells, respectively in the bone marrow at week 16 following engraftment in mice with control cells or CD33KO cells edited by the gRNA indicated (gRNAs from left to right on the X-axis: control, O, A or B).

FIGS. 25A-25D shows quantification of the percentage of hCD19+ cells, hCD14+ cells, hCD11b+ cells, or hCD3+ cells, respectively, at week 16 in the bone marrow, following engraftment in mice with control cells or CD33KO cells edited by the gRNA indicated (gRNAs from left to right on the X-axis: control, O, A or B).

FIGS. 26A-26B shows quantification of the percentage of hCD33+CD14+ cells or hCD33−CD14+ cells, respectively, in the bone marrow at week 16 following engraftment in mice with control cells or CD33KO cells edited by the gRNA indicated (gRNAs from left to right on the X-axis, control: O, A or B).

FIGS. 27A-D shows quantification of the percentage of hCD34+ cells, hCD38+ cells, hCD34+38− uncommitted progenitor cells, or hCD34+CD38+ committed progenitor cells, respectively, at week 16 in the bone marrow, following engraftment in mice with control cells or CD33KO cells edited by the gRNA indicated (gRNAs from left to right on the X-axis: control, O, A or B).

FIG. 28A demonstrates the percentage of edited cells in mice administered CD33KO cells that were edited with the following gRNAs: gRNA O (left panel), gRNA A (center panel), or gRNA B (right panel). FIGS. 28B-28D demonstrate the top 5 INDEL species representing different editing events observed in the isolated bone marrow cells for each gRNA used (gRNA O, gRNA A, and gRNA B, respectively) in generating the CD33KO cells. The 5 INDEL species from left to right on the X-axis for gRNA O are: −1 bp, −2 bp, +1 bp, −2 bp, and −5 bp. The 5 INDEL species from left to right on the X-axis for gRNA A are: −1 bp, +1 bp, −1 bp, −3 bp, and −2 bp. The 5 INDEL species from left to right on the X-axis for gRNA A are: +1 bp, −3 bp, −1 bp, −2 bp, and −1 bp.

FIGS. 29A-29F shows quantification of the percentage of hCD45+ cells, hCD33+ cells, hCD14+ cells, hCD11b+ cells, hCD19+ cells, or hCD3+ cells, respectively in the spleen at week 16 following engraftment in mice with control cells or CD33KO cells edited by the gRNA indicated (gRNAs from left to right on the X-axis: control, O, A or B).

FIGS. 30A-30C shows quantification of the percentage hCD11b+ cells, hCD33+CD11b+ cells, or hCD33−CD11b+ cells, respectively in the blood at week 16 following engraftment in mice with control cells or CD33KO cells edited by the gRNA indicated (gRNAs from left to right on the X-axis: control, O, A or B).

FIGS. 31A-31C shows quantification of the percentage hCD11b+ cells, hCD33+CD11b+ cells, or hCD33−CD11b+ cells, respectively in the bone marrow at week 16 following engraftment in mice with control cells or CD33KO cells edited by the gRNA indicated (gRNAs from left to right on the X-axis: control, O, A or B).

FIG. 32A shows quantification of the percentage of hCD123+ cells in the blood at week 16 following engraftment in mice with control cells or CD33KO cells edited by the gRNA indicated (gRNAs from left to right on the X-axis, control, O, A or B). FIG. 32B shows quantification of the percentage of hCD123+ cells (left) or hCD10+ cells (right) in the bone marrow at week 16 following engraftment in mice with control cells or CD33KO cells edited by the gRNA indicated (gRNAs from left to right on the X-axis: control, O, A or B).

### DETAILED DESCRIPTION OF THE INVENTION

### Definitions

The term “binds”, as used herein with reference to a gRNA interaction with a target domain, refers to the gRNA molecule and the target domain forming a complex. The complex may comprise two strands forming a duplex structure, or three or more strands forming a multi-stranded complex. The binding may constitute a step in a more extensive process, such as the cleavage of the target domain by a Cas endonuclease. In some embodiments, the gRNA binds to the target domain with perfect complementarity, and in other embodiments, the gRNA binds to the target domain with partial complementarity, e.g., with one or more mismatches. In some embodiments, when a gRNA binds to a target domain, the full targeting domain of the gRNA base pairs with the targeting domain. In other embodiments, only a portion of the target domain and/or only a portion of the targeting domain base pairs with the other. In an embodiment, the interaction is sufficient to mediate a target domain-mediated cleavage event.

A “Cas9 molecule” as that term is used herein, refers to a molecule or polypeptide that can interact with a gRNA and, in concert with the gRNA, home or localize to a site which comprises a target domain. Cas9 molecules include naturally occurring Cas9 molecules and engineered, altered, or modified Cas9 molecules that differ, e.g., by at least one amino acid residue, from a naturally occurring Cas9 molecule.

The terms “gRNA” and “guide RNA” are used interchangeably throughout and refer to a nucleic acid that promotes the specific targeting or homing of a gRNA/Cas9 molecule complex to a target nucleic acid. A gRNA can be unimolecular (having a single RNA molecule), sometimes referred to herein as sgRNAs, or modular (comprising more than one, and typically two, separate RNA molecules). A gRNA may bind to a target domain in the genome of a host cell. The gRNA (e.g., the targeting domain thereof) may be partially or completely complementary to the target domain. The gRNA may also comprise a “scaffold sequence,” (e.g., a tracrRNA sequence), that recruits a Cas9 molecule to a target domain bound to a gRNA sequence (e.g., by the targeting domain of the gRNA sequence). The scaffold sequence may comprise at least one stem loop structure and recruits an endonuclease. Exemplary scaffold sequences can be found, for example, in Jinek, et al. Science (2012) 337(6096):816-821, Ran, et al. Nature Protocols (2013) 8:2281-2308, PCT Application No. WO2014/093694, and PCT Application No. WO2013/176772.

The term “mutation” is used herein to refer to a genetic change (e.g., insertion, deletion, or substitution) in a nucleic acid compared to a reference sequence, e.g., the corresponding wild-type nucleic acid. In some embodiments, a mutation to a gene detargetizes the protein produced by the gene. In some embodiments, a detargetized CD33 protein is not bound by, or is bound at a lower level by, an agent that targets CD33.

The “targeting domain” of the gRNA is complementary to the “target domain” on the target nucleic acid. The strand of the target nucleic acid comprising the nucleotide sequence complementary to the core domain of the gRNA is referred to herein as the “complementary strand” of the target nucleic acid. Guidance on the selection of targeting domains can be found, e.g., in Fu Y et al, Nat Biotechnol 2014 (doi: 10.1038/nbt.2808) and Sternberg S H et al., Nature 2014 (doi: 10.1038/nature13011).

### Nucleases

In some embodiments, a cell (e.g., HSC or HPC) described herein is made using a nuclease described herein. Exemplary nucleases include Cas molecules (e.g., Cas9 or Cas12a), TALENs, ZFNs, and meganucleases. In some embodiments, a nuclease is used in combination with a CD33 gRNA described herein (e.g., according to Table 2).

Cas9 Molecules

In some embodiments, a CD33 gRNA described herein is complexed with a Cas9 molecule. Various Cas9 molecules can be used. In some embodiments, a Cas9 molecule is selected that has the desired PAM specificity to target the gRNA/Cas9 molecule complex to the target domain in CD33. In some embodiments, genetically engineering a cell also comprises introducing one or more (e.g., 1, 2, 3 or more) Cas9 molecules into the cell.

Cas9 molecules of a variety of species can be used in the methods and compositions described herein. In embodiments, the Cas9 molecule is of, or derived from, S. pyogenes (SpCas9), S. aureus (SaCas9) or S. thermophilus. Additional suitable Cas9 molecules include those of, or derived from, Staphylococcus aureus, Neisseria meningitidis (NmCas9), Acidovorax avenae, Actinobacillus pleuropneumoniae, Actinobacillus succinogenes, Actinobacillus suis, Actinomyces sp., cycliphilus denitrificans, Aminomonas paucivorans, Bacillus cereus, Bacillus smithii, Bacillus thuringiensis, Bacteroides sp., Blastopirellula marina, Bradyrhizobium sp., Brevibacillus laterosporus, Campylobacter coli, Campylobacter jejuni (CjCas9), Campylobacter lari, Candidatus Puniceispirillum, Clostridium cellulolyticum, Clostridium perfringens, Corynebacterium accolens, Corynebacterium diphtheria, Corynebacterium matruchotii, Dinoroseobacter shibae, Eubacterium dolichum, gamma proteobacterium, Gluconacetobacter diazotrophicus, Haemophilus parainfluenzae, Haemophilus sputorum, Helicobacter canadensis, Helicobacter cinaedi, Helicobacter mustelae, Ilyobacter polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, Neisseria meningitidis, Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus lugdunensis, Streptococcus sp., Subdoligranulum sp., Tistrella mobilis, Treponema sp., or Verminephrobacter eiseniae.

In some embodiments, the Cas9 molecule is a naturally occurring Cas9 molecule. In some embodiments, the Cas9 molecule is an engineered, altered, or modified Cas9 molecule that differs, e.g., by at least one amino acid residue, from a reference sequence, e.g., the most similar naturally occurring Cas9 molecule or a sequence of Table 50 of WO2015157070, which is herein incorporated by reference in its entirety.

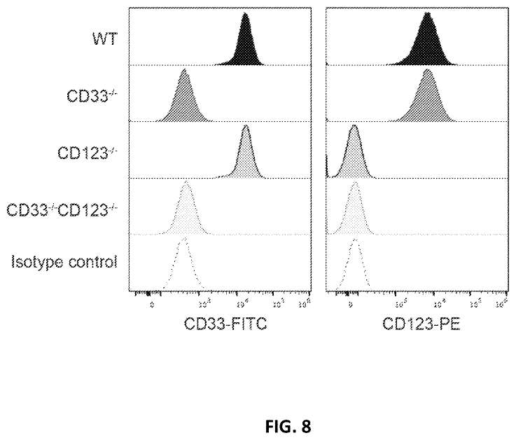

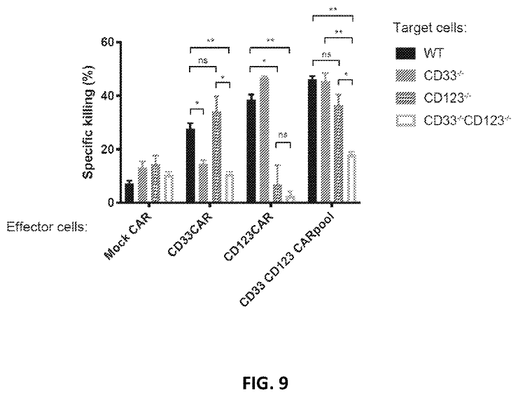

A naturally occurring Cas9 molecule typically comprises two lobes: a recognition (REC) lobe and a nuclease (NUC) lobe; each of which further comprises domains described, e.g., in WO2015157070, e.g., in FIGS. 9A-9B therein (which application is incorporated herein by reference in its entirety).

The REC lobe comprises the arginine-rich bridge helix (BH), the REC1 domain, and the REC2 domain. The REC lobe appears to be a Cas9-specific functional domain. The BH domain is a long alpha helix and arginine rich region and comprises amino acids 60-93 of the sequence of S. pyogenes Cas9. The REC1 domain is involved in recognition of the repeat: anti-repeat duplex, e.g., of a gRNA or a tracrRNA. The REC1 domain comprises two REC1 motifs at amino acids 94 to 179 and 308 to 717 of the sequence of S. pyogenes Cas9. These two REC1 domains, though separated by the REC2 domain in the linear primary structure, assemble in the tertiary structure to form the REC1 domain. The REC2 domain, or parts thereof, may also play a role in the recognition of the repeat: anti-repeat duplex. The REC2 domain comprises amino acids 180-307 of the sequence of S. pyogenes Cas9.

The NUC lobe comprises the RuvC domain (also referred to herein as RuvC-like domain), the HNH domain (also referred to herein as HNH-like domain), and the PAM-interacting (PI) domain. The RuvC domain shares structural similarity to retroviral integrase superfamily members and cleaves a single strand, e.g., the non-complementary strand of the target nucleic acid molecule. The RuvC domain is assembled from the three split RuvC motifs (RuvC I, RuvCII, and RuvCIII, which are often commonly referred to in the art as RuvCI domain, or N-terminal RuvC domain, RuvCII domain, and RuvCIII domain) at amino acids 1-59, 718-769, and 909-1098, respectively, of the sequence of S. pyogenes Cas9. Similar to the REC1 domain, the three RuvC motifs are linearly separated by other domains in the primary structure, however in the tertiary structure, the three RuvC motifs assemble and form the RuvC domain. The HNH domain shares structural similarity with HNH endonucleases, and cleaves a single strand, e.g., the complementary strand of the target nucleic acid molecule. The HNH domain lies between the RuvC II-III motifs and comprises amino acids 775-908 of the sequence of S. pyogenes Cas9. The PI domain interacts with the PAM of the target nucleic acid molecule, and comprises amino acids 1099-1368 of the sequence of S. pyogenes Cas9.

Crystal structures have been determined for naturally occurring bacterial Cas9 molecules (Jinek et al., Science, 343(6176): 1247997, 2014) and for S. pyogenes Cas9 with a guide RNA (e.g., a synthetic fusion of crRNA and tracrRNA) (Nishimasu et al., Cell, 156:935-949, 2014; and Anders et al., Nature, 2014, doi: 10.1038/nature13579).

In some embodiments, a Cas9 molecule described herein has nuclease activity, e.g., double strand break activity. In some embodiments, the Cas9 molecule has been modified to inactivate one of the catalytic residues of the endonuclease. In some embodiments, the Cas9 molecule is a nickase and produces a single stranded break. See, e.g., Dabrowska et al. Frontiers in Neuroscience (2018) 12(75). It has been shown that one or more mutations in the RuvC and HNH catalytic domains of the enzyme may improve Cas9 efficiency. See, e.g., Sarai et al. Currently Pharma. Biotechnol. (2017) 18(13). In some embodiments, the Cas9 molecule is fused to a second domain, e.g., a domain that modifies DNA or chromatin, e.g., a deaminase or demethylase domain. In some such embodiments, the Cas9 molecule is modified to eliminate its endonuclease activity.

In some embodiments, a Cas9 molecule described herein is administered together with a template for homology directed repair (HDR). In some embodiments, a Cas9 molecule described herein is administered without a HDR template.

In some embodiments, the Cas9 molecule is modified to enhance specificity of the enzyme (e.g., reduce off-target effects, maintain robust on-target cleavage). In some embodiments, the Cas9 molecule is an enhanced specificity Cas9 variant (e.g., eSPCas9). See, e.g., Slaymaker et al. Science (2016) 351 (6268): 84-88. In some embodiments, the Cas9 molecule is a high fidelity Cas9 variant (e.g., SpCas9-HF1). See, e.g., Kleinstiver et al. Nature (2016) 529: 490-495.

Various Cas9 molecules are known in the art and may be obtained from various sources and/or engineered/modified to modulate one or more activities or specificities of the enzymes. In some embodiments, the Cas9 molecule has been engineered/modified to recognize one or more PAM sequence. In some embodiments, the Cas9 molecule has been engineered/modified to recognize one or more PAM sequence that is different than the PAM sequence the Cas9 molecule recognizes without engineering/modification. In some embodiments, the Cas9 molecule has been engineered/modified to reduce off-target activity of the enzyme.

In some embodiments, the nucleotide sequence encoding the Cas9 molecule is modified further to alter the specificity of the endonuclease activity (e.g., reduce off-target cleavage, decrease the endonuclease activity or lifetime in cells, increase homology-directed recombination and reduce non-homologous end joining). See, e.g., Komor et al. Cell (2017) 168: 20-36. In some embodiments, the nucleotide sequence encoding the Cas9 molecule is modified to alter the PAM recognition of the endonuclease. For example, the Cas9 molecule SpCas9 recognizes PAM sequence NGG, whereas relaxed variants of the SpCas9 comprising one or more modifications of the endonuclease (e.g., VQR SpCas9, EQR SpCas9, VRER SpCas9) may recognize the PAM sequences NGA, NGAG, NGCG. PAM recognition of a modified Cas9 molecule is considered “relaxed” if the Cas9 molecule recognizes more potential PAM sequences as compared to the Cas9 molecule that has not been modified. For example, the Cas9 molecule SaCas9 recognizes PAM sequence NNGRRT, whereas a relaxed variant of the SaCas9 comprising one or more modifications (e.g., KKH SaCas9) may recognize the PAM sequence NNNRRT. In one example, the Cas9 molecule FnCas9 recognizes PAM sequence NNG, whereas a relaxed variant of the FnCas9 comprising one or more modifications of the endonuclease (e.g., RHA FnCas9) may recognize the PAM sequence YG. In one example, the Cas9 molecule is a Cpf1 endonuclease comprising substitution mutations S542R and K607R and recognize the PAM sequence TYCV. In one example, the Cas9 molecule is a Cpf1 endonuclease comprising substitution mutations S542R, K607R, and N552R and recognize the PAM sequence TATV. See, e.g., Gao et al. Nat. Biotechnol. (2017) 35(8): 789-792.

In some embodiments, more than one (e.g., 2, 3, or more) Cas molecules, e.g., Cas9 molecules, are used. In some embodiments, at least one of the Cas9 molecule is a Cas9 enzyme. In some embodiments, at least one of the Cas molecules is a Cpf1 enzyme. In some embodiments, at least one of the Cas9 molecule is derived from Streptococcus pyogenes. In some embodiments, at least one of the Cas9 molecule is derived from Streptococcus pyogenes and at least one Cas9 molecule is derived from an organism that is not Streptococcus pyogenes. In some embodiments, the Cas9 molecule is a base editor. Base editor endonuclease generally comprises a catalytically inactive Cas9 molecule fused to a function domain. See, e.g., Eid et al. Biochem. J. (2018) 475(11): 1955-1964; Rees et al. Nature Reviews Genetics (2018) 19:770-788. In some embodiments, the catalytically inactive Cas9 molecule is dCas9. In some embodiments, the, the catalytically inactive Cas9 molecule (dCas9) is fused to one or more uracil glycosylase inhibitor (UGI) domains. In some embodiments, the endonuclease comprises a dCas9 fused to an adenine base editor (ABE), for example an ABE evolved from the RNA adenine deaminase TadA. In some embodiments, the endonuclease comprises a dCas9 fused to cytidine deaminase enzyme (e.g., APOBEC deaminase, pmCDA1, activation-induced cytidine deaminase (AID)). In some embodiments, the catalytically inactive Cas9 molecule has reduced activity and is nCas9. In some embodiments, the Cas9 molecule comprises a nCas9 fused to one or more uracil glycosylase inhibitor (UGI) domains. In some embodiments, the Cas9 molecule comprises a nCas9 fused to an adenine base editor (ABE), for example an ABE evolved from the RNA adenine deaminase TadA. In some embodiments, the Cas9 molecule comprises a nCas9 fused to cytidine deaminase enzyme (e.g., APOBEC deaminase, pmCDA1, activation-induced cytidine deaminase (AID)).

Examples of base editors include, without limitation, BE1, BE2, BE3, HF-BE3, BE4, BE4max, BE4-Gam, YE1-BE3, EE-BE3, YE2-BE3, YEE-CE3, VQR-BE3, VRER-BE3, SaBE3, SaBE4, SaBE4-Gam, Sa(KKH)-BE3, Target-AID, Target-AID-NG, xBE3, eA3A-BE3, BE-PLUS, TAM, CRISPR-X, ABE7.9, ABE7.10, ABE7.10*, xABE, ABESa, VQR-ABE, VRER-ABE, Sa(KKH)-ABE, and CRISPR-SKIP. Additional examples of base editors can be found, for example, in US Publication No. 2018/0312825A1, US Publication No. 2018/0312828A1, and PCT Publication No. WO 2018/165629A1, which are incorporated by reference herein in their entireties.

In some embodiments, the base editor has been further modified to inhibit base excision repair at the target site and induce cellular mismatch repair. Any of the Cas9 molecules described herein may be fused to a Gam domain (bacteriophage Mu protein) to protect the Cas9 molecule from degradation and exonuclease activity. See, e.g., Eid et al. Biochem. J. (2018) 475(11): 1955-1964.

In some embodiments, the Cas9 molecule belongs to class 2 type V of Cas endonuclease. Class 2 type V Cas endonucleases can be further categorized as type V-A, type V-B, type V-C, and type V-U. See, e.g., Stella et al. Nature Structural & Molecular Biology (2017). In some embodiments, the Cas molecule is a type V-A Cas endonuclease, such as a Cpf1 nuclease. In some embodiments, the Ca Cas9 molecule is a type V-B Cas endonuclease, such as a C2c1 endonuclease. See, e.g., Shmakov et al. Mol Cell (2015) 60: 385-397. In some embodiments, the Cas molecule is Mad7. Alternatively or in addition, the Cas9 molecule is a Cpf1 nuclease or a variant thereof. As will be appreciated by one of skill in the art, the Cpf1 nuclease may also be referred to as Cas12a. See, e.g., Strohkendl et al. Mol. Cell (2018) 71: 1-9. In some embodiments, a composition or method described herein involves, or a host cell expresses, a Cpf1 nuclease derived from Provetella spp. or Francisella spp., Acidaminococcus sp. (AsCpf1), Lachnospiraceae bacterium (LpCpf1), or Eubacterium rectale. In some embodiments, the nucleotide sequence encoding the Cpf1 nuclease may be codon optimized for expression in a host cell. In some embodiments, the nucleotide sequence encoding the Cpf1 endonuclease is further modified to alter the activity of the protein.

In some embodiments, catalytically inactive variants of Cas molecules (e.g., of Cas9 or Cas12a) are used according to the methods described herein. A catalytically inactive variant of Cpf1 (Cas12a) may be referred to dCas12a. As described herein, catalytically inactive variants of Cpf1 maybe fused to a function domain to form a base editor. See, e.g., Rees et al. Nature Reviews Genetics (2018) 19:770-788. In some embodiments, the catalytically inactive Cas9 molecule is dCas9. In some embodiments, the endonuclease comprises a dCas12a fused to one or more uracil glycosylase inhibitor (UGI) domains. In some embodiments, the Cas9 molecule comprises a dCas12a fused to an adenine base editor (ABE), for example an ABE evolved from the RNA adenine deaminase TadA. In some embodiments, the Cas molecule comprises a dCas12a fused to cytidine deaminase enzyme (e.g., APOBEC deaminase, pmCDA1, activation-induced cytidine deaminase (AID)).

Alternatively or in addition, the Cas9 molecule may be a Cas14 endonuclease or variant thereof. Cas14 endonucleases are derived from archaea and tend to be smaller in size (e.g., 400-700 amino acids). Additionally Cas14 endonucleases do not require a PAM sequence. See, e.g., Harrington et al. Science (2018).

Any of the Cas9 molecules described herein may be modulated to regulate levels of expression and/or activity of the Cas9 molecule at a desired time. For example, it may be advantageous to increase levels of expression and/or activity of the Cas9 molecule during particular phase(s) of the cell cycle. It has been demonstrated that levels of homology-directed repair are reduced during the G1 phase of the cell cycle, therefore increasing levels of expression and/or activity of the Cas9 molecule during the S phase, G2 phase, and/or M phase may increase homology-directed repair following the Cas endonuclease editing. In some embodiments, levels of expression and/or activity of the Cas9 molecule are increased during the S phase, G2 phase, and/or M phase of the cell cycle. In one example, the Cas9 molecule fused to the N-terminal region of human Geminin. See, e.g., Gutschner et al. Cell Rep. (2016) 14(6): 1555-1566. In some embodiments, levels of expression and/or activity of the Cas9 molecule are reduced during the G1 phase. In one example, the Cas9 molecule is modified such that it has reduced activity during the G1 phase. See, e.g., Lomova et al. Stem Cells (2018).

Alternatively or in addition, any of the Cas9 molecules described herein may be fused to an epigenetic modifier (e.g., a chromatin-modifying enzyme, e.g., DNA methylase, histone deacetylase). See, e.g., Kungulovski et al. Trends Genet. (2016) 32(2):101-113. Cas9 molecule fused to an epigenetic modifier may be referred to as “epieffectors” and may allow for temporal and/or transient endonuclease activity. In some embodiments, the Cas9 molecule is a dCas9 fused to a chromatin-modifying enzyme.

Zinc Finger Nucleases

In some embodiments, a cell or cell population described herein is produced using zinc finger (ZFN) technology. In some embodiments, the ZFN recognizes a target domain described herein, e.g., in Table 1. In general, zinc finger mediated genomic editing involves use of a zinc finger nuclease, which typically comprises a zinc finger DNA binding domain and a nuclease domain. The zinc finger binding domain may be engineered to recognize and bind to any target domain of interest, e.g., may be designed to recognize a DNA sequence ranging from about 3 nucleotides to about 21 nucleotides in length, or from about 8 to about 19 nucleotides in length. Zinc finger binding domains typically comprise at least three zinc finger recognition regions (e.g., zinc fingers).

Restriction endonucleases (restriction enzymes) capable of sequence-specific binding to DNA (at a recognition site) and cleaving DNA at or near the site of binding are known in the art and may be used to form ZFN for use in genomic editing. For example, Type IIS restriction endonucleases cleave DNA at sites removed from the recognition site and have separable binding and cleavage domains. In one example, the DNA cleavage domain may be derived from the FokI endonuclease.

TALENs

In some embodiments, a cell or cell population described herein is produced using TALEN technology. In some embodiments, the TALEN recognizes a target domain described herein, e.g., in Table 1. In general, TALENs are engineered restriction enzymes that can specifically bind and cleave a desired target DNA molecule. A TALEN typically contains a Transcriptional Activator-Like Effector (TALE) DNA-binding domain fused to a DNA cleavage domain. The DNA binding domain may contain a highly conserved 33-34 amino acid sequence with a divergent 2 amino acid RVD (repeat variable dipeptide motif) at positions 12 and 13. The RVD motif determines binding specificity to a nucleic acid sequence and can be engineered to specifically bind a desired DNA sequence. In one example, the DNA cleavage domain may be derived from the FokI endonuclease. In some embodiments, the FokI domain functions as a dimer, using two constructs with unique DNA binding domains for sites in the target genome with proper orientation and spacing.

A TALEN specific to a target gene of interest can be used inside a cell to produce a double-stranded break (DSB). A mutation can be introduced at the break site if the repair mechanisms improperly repair the break via non-homologous end joining. For example, improper repair may introduce a frame shift mutation. Alternatively, a foreign DNA molecule having a desired sequence can be introduced into the cell along with the TALEN. Depending on the sequence of the foreign DNA and chromosomal sequence, this process can be used to correct a defect or introduce a DNA fragment into a target gene of interest, or introduce such a defect into the endogenous gene, thus decreasing expression of the target gene.

Some exemplary, non-limiting embodiments of endonucleases and nuclease variants suitable for use in connection with the guide RNAs and genetic engineering methods provided herein have been described above. Additional suitable nucleases and nuclease variants will be apparent to those of skill in the art based on the present disclosure and the knowledge in the art. The disclosure is not limited in this respect.

gRNA Sequences and Configurations

gRNA Configuration Generally

A gRNA can comprise a number of domains. In an embodiment, a unimolecular, sgRNA, or chimeric, gRNA comprises, e.g., from 5′ to 3′:
a targeting domain (which is complementary to a target nucleic acid in the CD33 gene;
a first complementarity domain;
a linking domain;
a second complementarity domain (which is complementary to the first complementarity domain);
a proximal domain; and
optionally, a tail domain.

Each of these domains is now described in more detail.

The targeting domain may comprise a nucleotide sequence that is complementary, e.g., at least 80, 85, 90, or 95% complementary, e.g., fully complementary, to the target sequence on the target nucleic acid. The targeting domain is part of an RNA molecule and will therefore comprise the base uracil (U), while any DNA encoding the gRNA molecule will comprise the base thymine (T). While not wishing to be bound by theory, in an embodiment, it is believed that the complementarity of the targeting domain with the target sequence contributes to specificity of the interaction of the gRNA/Cas9 molecule complex with a target nucleic acid. It is understood that in a targeting domain and target sequence pair, the uracil bases in the targeting domain will pair with the adenine bases in the target sequence. In an embodiment, the target domain itself comprises in the 5′ to 3′ direction, an optional secondary domain, and a core domain. In an embodiment, the core domain is fully complementary with the target sequence. In an embodiment, the targeting domain is 5 to 50 nucleotides in length. The targeting domain may be between 15-25 nucleotides, 18-22 nucleotides, or 19-21 nucleotides in length. In some embodiments, the targeting domain is 15, 16, 17, 18, 19, 20, 21, 22, 23, 24, or 25 nucleotides in length. In some embodiments, the targeting domain is between 10-30, or between 15-25, nucleotides in length.

In some embodiments, a targeting domain comprises a core domain and a secondary targeting domain, e.g., as described in International Application WO2015157070, which is incorporated by reference in its entirety. In an embodiment, the core domain comprises about 8 to about 13 nucleotides from the 3′ end of the targeting domain (e.g., the most 3′ 8 to 13 nucleotides of the targeting domain). In an embodiment, the secondary domain is positioned 5′ to the core domain. In many embodiments, the core domain has exact complementarity with the corresponding region of the target sequence. In other embodiments, the core domain can have 1 or more nucleotides that are not complementary with the corresponding nucleotide of the target sequence.

The first complementarity domain is complementary with the second complementarity domain, and in an embodiment, has sufficient complementarity to the second complementarity domain to form a duplexed region under at least some physiological conditions. In an embodiment, the first complementarity domain is 5 to 30 nucleotides in length. In an embodiment, the first complementarity domain comprises 3 subdomains, which, in the 5′ to 3′ direction are: a 5′ subdomain, a central subdomain, and a 3′ subdomain. In an embodiment, the 5′ subdomain is 4 to 9, e.g., 4, 5, 6, 7, 8 or 9 nucleotides in length. In an embodiment, the central subdomain is 1, 2, or 3, e.g., 1, nucleotide in length. In an embodiment, the 3′ subdomain is 3 to 25, e.g., 4 to 22, 4 to 18, or 4 to 10, or 3, 4, 5, 6, 7, 8, 9, 10, 11, 12, 13, 14, 15, 16, 17, 18, 19, 20, 21, 22, 23, 24, or 25 nucleotides in length. The first complementarity domain can share homology with, or be derived from, a naturally occurring first complementarity domain. In an embodiment, it has at least 50% homology with a S. pyogenes, S. aureus or S. thermophilus, first complementarity domain.

The sequence and placement of the above-mentioned domains are described in more detail in WO2015157070, which is herein incorporated by reference in its entirety, including p. 88-112 therein.

A linking domain serves to link the first complementarity domain with the second complementarity domain of a unimolecular gRNA. The linking domain can link the first and second complementarity domains covalently or non-covalently. In an embodiment, the linkage is covalent. In an embodiment, the linking domain is, or comprises, a covalent bond interposed between the first complementarity domain and the second complementarity domain. In some embodiments, the linking domain comprises one or more, e.g., 2, 3, 4, 5, 6, 7, 8, 9, or 10 nucleotides. In some embodiments, the linking domain comprises at least one non-nucleotide bond, e.g., as disclosed in WO2018126176, the entire contents of which are incorporated herein by reference.

The second complementarity domain is complementary, at least in part, with the first complementarity domain, and in an embodiment, has sufficient complementarity to the second complementarity domain to form a duplexed region under at least some physiological conditions. In an embodiment, the second complementarity domain can include a sequence that lacks complementarity with the first complementarity domain, e.g., a sequence that loops out from the duplexed region. In an embodiment, the second complementarity domain is 5 to 27 nucleotides in length. In an embodiment, the second complementarity domain is longer than the first complementarity region. In an embodiment, the complementary domain is 5, 6, 7, 8, 9, 10, 11, 12, 13, 14, 15, 16, 17, 18, 19, 20, 21, 22, 23, 24 or 25 nucleotides in length. In an embodiment, the second complementarity domain comprises 3 subdomains, which, in the 5′ to 3′ direction are: a 5′ subdomain, a central subdomain, and a 3′ subdomain. In an embodiment, the 5′ subdomain is 3 to 25, e.g., 4 to 22, 4 to 18, or 4 to 10, or 3, 4, 5, 6, 7, 8, 9, 10, 11, 12, 13, 14, 15, 16, 17, 18, 19, 20, 21, 22, 23, 24, or 25 nucleotides in length. In an embodiment, the central subdomain is 1, 2, 3, 4 or 5, e.g., 3, nucleotides in length. In an embodiment, the 3′ subdomain is 4 to 9, e.g., 4, 5, 6, 7, 8 or 9 nucleotides in length. In an embodiment, the 5′ subdomain and the 3′ subdomain of the first complementarity domain, are respectively, complementary, e.g., fully complementary, with the 3′ subdomain and the 5′ subdomain of the second complementarity domain.

In an embodiment, the proximal domain is 5 to 20 nucleotides in length. In an embodiment, the proximal domain can share homology with or be derived from a naturally occurring proximal domain. In an embodiment, it has at least 50% homology with an S. pyogenes, S. aureus or S. thermophilus, proximal domain.

A broad spectrum of tail domains are suitable for use in gRNsA. In an embodiment, the tail domain is 0 (absent), 1, 2, 3, 4, 5, 6, 7, 8, 9, or 10 nucleotides in length. In some embodiments, the tail domain nucleotides are from or share homology with a sequence from the 5′ end of a naturally occurring tail domain. In an embodiment, the tail domain includes sequences that are complementary to each other and which, under at least some physiological conditions, form a duplexed region. In an embodiment, the tail domain is absent or is 1 to 50 nucleotides in length. In an embodiment, the tail domain can share homology with or be derived from a naturally occurring proximal tail domain. In an embodiment, it has at least 50% homology with an S. pyogenes, S. aureus or S. thermophilus, tail domain. In an embodiment, the tail domain includes nucleotides at the 3′ end that are related to the method of in vitro or in vivo transcription.

In some embodiments, modular gRNA comprises:
a first strand comprising, e.g., from 5′ to 3′;
   a targeting domain (which is complementary to a target nucleic acid in the CD33 gene) and
   a first complementarity domain; and
a second strand, comprising, preferably from 5′ to 3′:
   optionally, a 5′ extension domain;
   a second complementarity domain;
   a proximal domain; and
   optionally, a tail domain.

In some embodiments, the gRNA is chemically modified. For instance, the gRNA may comprise one or more modification chosen from phosphorothioate backbone modification, 2′-O-Me-modified sugars (e.g., at one or both of the 3′ and 5′ termini), 2′F-modified sugar, replacement of the ribose sugar with the bicyclic nucleotide-cEt, 3′thioPACE (MSP), or any combination thereof. Suitable gRNA modifications are described, e.g., in Randar et al. PNAS Dec. 22, 2015 112 (51) E7110-E7117 and Hendel et al., Nat Biotechnol. 2015 September; 33(9): 985-989, each of which is incorporated herein by reference in its entirety. In some embodiments, a gRNA described herein comprises one or more 2′-O-methyl-3′-phosphorothioate nucleotides, e.g., at least 2, 3, 4, 5, or 6 2′-O-methyl-3′-phosphorothioate nucleotides. In some embodiments, a gRNA described herein comprises modified nucleotides (e.g., 2′-O-methyl-3′-phosphorothioate nucleotides) at the three terminal positions and the 5′ end and/or at the three terminal positions and the 3′ end. In some embodiments, the gRNA may comprise one or more modified nucleotides, e.g., as described in International Applications WO/2017/214460, WO/2016/089433, and WO/2016/164356, which are incorporated by reference their entirety.

In some embodiments, a gRNA described herein is chemically modified. For example, the gRNA may comprise one or more 2′-O modified nucleotide, e.g., 2′-O-methyl nucleotide. In some embodiments, the gRNA comprises a 2′-O modified nucleotide, e.g., 2′-O-methyl nucleotide at the 5′ end of the gRNA. In some embodiments, the gRNA comprises a 2′-O modified nucleotide, e.g., 2′-O-methyl nucleotide at the 3′ end of the gRNA. In some embodiments, the gRNA comprises a 2′-O-modified nucleotide, e.g., 2′-O-methyl nucleotide at both the 5′ and 3′ ends of the gRNA. In some embodiments, the gRNA is 2′-O-modified, e.g. 2′-O-methyl-modified at the nucleotide at the 5′ end of the gRNA, the second nucleotide from the 5′ end of the gRNA, and the third nucleotide from the 5′ end of the gRNA. In some embodiments, the gRNA is 2′-O-modified, e.g. 2′-O-methyl-modified at the nucleotide at the 3′ end of the gRNA, the second nucleotide from the 3′ end of the gRNA, and the third nucleotide from the 3′ end of the gRNA. In some embodiments, the gRNA is 2′-O-modified, e.g. 2′-O-methyl-modified at the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA. In some embodiments, the gRNA is 2′-O-modified, e.g. 2′-O-methyl-modified at the second nucleotide from the 3′ end of the gRNA, the third nucleotide from the 3′ end of the gRNA, and at the fourth nucleotide from the 3′ end of the gRNA. In some embodiments, the nucleotide at the 3′ end of the gRNA is not chemically modified. In some embodiments, the nucleotide at the 3′ end of the gRNA does not have a chemically modified sugar. In some embodiments, the gRNA is 2′-O-modified, e.g. 2′-O-methyl-modified, at the nucleotide at the 5′ end of the gRNA, the second nucleotide from the 5′ end of the gRNA, the third nucleotide from the 5′ end of the gRNA, the second nucleotide from the 3′ end of the gRNA, the third nucleotide from the 3′ end of the gRNA, and the fourth nucleotide from the 3′ end of the gRNA. In some embodiments, the 2′-O-methyl nucleotide comprises a phosphate linkage to an adjacent nucleotide. In some embodiments, the 2′-O-methyl nucleotide comprises a phosphorothioate linkage to an adjacent nucleotide. In some embodiments, the 2′-O-methyl nucleotide comprises a thioPACE linkage to an adjacent nucleotide.

In some embodiments, the gRNA may comprise one or more 2′-O-modified and 3′phosphorous-modified nucleotide, e.g., a 2′-O-methyl 3′phosphorothioate nucleotide. In some embodiments, the gRNA comprises a 2′-O-modified and 3′phosphorous-modified, e.g., 2′-O-methyl 3′phosphorothioate nucleotide at the 5′ end of the gRNA. In some embodiments, the gRNA comprises a 2′-O-modified and 3′phosphorous-modified, e.g., 2′-O-methyl 3′phosphorothioate nucleotide at the 3′ end of the gRNA. In some embodiments, the gRNA comprises a 2′-O-modified and 3′phosphorous-modified, e.g., 2′-O-methyl 3′phosphorothioate nucleotide at the 5′ and 3′ ends of the gRNA. In some embodiments, the gRNA comprises a backbone in which one or more non-bridging oxygen atoms has been replaced with a sulfur atom. In some embodiments, the gRNA is 2′-O-modified and 3′phosphorous-modified, e.g. 2′-O-methyl 3′phosphorothioate-modified at the nucleotide at the 5′ end of the gRNA, the second nucleotide from the 5′ end of the gRNA, and the third nucleotide from the 5′ end of the gRNA. In some embodiments, the gRNA is 2′-O-modified and 3′phosphorous-modified, e.g. 2′-O-methyl 3′phosphorothioate-modified at the nucleotide at the 3′ end of the gRNA, the second nucleotide from the 3′ end of the gRNA, and the third nucleotide from the 3′ end of the gRNA. In some embodiments, the gRNA is 2′-O-modified and 3′phosphorous-modified, e.g. 2′-O-methyl 3′phosphorothioate-modified at the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA. In some embodiments, the gRNA is 2′-O-modified and 3′phosphorous-modified, e.g. 2′-O-methyl 3′phosphorothioate-modified at the second nucleotide from the 3′ end of the gRNA, the third nucleotide from the 3′ end of the gRNA, and the fourth nucleotide from the 3′ end of the gRNA. In some embodiments, the nucleotide at the 3′ end of the gRNA is not chemically modified. In some embodiments, the nucleotide at the 3′ end of the gRNA does not have a chemically modified sugar. In some embodiments, the gRNA is 2′-O-modified and 3′phosphorous-modified, e.g. 2′-O-methyl 3′phosphorothioate-modified at the nucleotide at the 5′ end of the gRNA, the second nucleotide from the 5′ end of the gRNA, the third nucleotide from the 5′ end of the gRNA, the second nucleotide from the 3′ end of the gRNA, the third nucleotide from the 3′ end of the gRNA, and the fourth nucleotide from the 3′ end of the gRNA.

In some embodiments, the gRNA may comprise one or more 2′-O-modified and 3′-phosphorous-modified, e.g., 2′-O-methyl 3′thioPACE nucleotide. In some embodiments, the gRNA comprises a 2′-O-modified and 3′phosphorous-modified, e.g., 2′-O-methyl 3′thioPACE nucleotide at the 5′ end of the gRNA. In some embodiments, the gRNA comprises a 2′-O-modified and 3′phosphorous-modified, e.g., 2′-O-methyl 3′thioPACE nucleotide at the 3′ end of the gRNA. In some embodiments, the gRNA comprises a 2′-O-modified and 3′phosphorous-modified, e.g., 2′-O-methyl 3′thioPACE nucleotide at the 5′ and 3′ ends of the gRNA. In some embodiments, the gRNA comprises a backbone in which one or more non-bridging oxygen atoms have been replaced with a sulfur atom and one or more non-bridging oxygen atoms have been replaced with an acetate group. In some embodiments, the gRNA is 2′-O-modified and 3′phosphorous-modified, e.g. 2′-O-methyl 3′ thioPACE-modified at the nucleotide at the 5′ end of the gRNA, the second nucleotide from the 5′ end of the gRNA, and the third nucleotide from the 5′ end of the gRNA. In some embodiments, the gRNA is 2′-O-modified and 3′phosphorous-modified, e.g. 2′-O-methyl 3′thioPACE-modified at the nucleotide at the 3′ end of the gRNA, the second nucleotide from the 3′ end of the gRNA, and the third nucleotide from the 3′ end of the gRNA. In some embodiments, the gRNA is 2′-O-modified and 3′phosphorous-modified, e.g. 2′-O-methyl 3′thioPACE-modified at the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA. In some embodiments, the gRNA is 2′-O-modified and 3′phosphorous-modified, e.g. 2′-O-methyl 3′thioPACE-modified at the second nucleotide from the 3′ end of the gRNA, the third nucleotide from the 3′ end of the gRNA, and the fourth nucleotide from the 3′ end of the gRNA. In some embodiments, the nucleotide at the 3′ end of the gRNA is not chemically modified. In some embodiments, the nucleotide at the 3′ end of the gRNA does not have a chemically modified sugar. In some embodiments, the gRNA is 2′-O-modified and 3′phosphorous-modified, e.g. 2′-O-methyl 3′thioPACE-modified at the nucleotide at the 5′ end of the gRNA, the second nucleotide from the 5′ end of the gRNA, the third nucleotide from the 5′ end of the gRNA, the second nucleotide from the 3′ end of the gRNA, the third nucleotide from the 3′ end of the gRNA, and the fourth nucleotide from the 3′ end of the gRNA.

In some embodiments, the gRNA comprises a chemically modified backbone. In some embodiments, the gRNA comprises a phosphorothioate linkage. In some embodiments, one or more non-bridging oxygen atoms have been replaced with a sulfur atom. In some embodiments, the nucleotide at the 5′ end of the gRNA, the second nucleotide from the 5′ end of the gRNA, and the third nucleotide from the 5′ end of the gRNA each comprise a phosphorothioate linkage. In some embodiments, the nucleotide at the 3′ end of the gRNA, the second nucleotide from the 3′ end of the gRNA, and the third nucleotide from the 3′ end of the gRNA each comprise a phosphorothioate linkage. In some embodiments, the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA each comprise a phosphorothioate linkage. In some embodiments, the second nucleotide from the 3′ end of the gRNA, the third nucleotide from the 3′ end of the gRNA, and at the fourth nucleotide from the 3′ end of the gRNA each comprise a phosphorothioate linkage. In some embodiments, the nucleotide at the 5′ end of the gRNA, the second nucleotide from the 5′ end of the gRNA, the third nucleotide from the 5′ end, the second nucleotide from the 3′ end of the gRNA, the third nucleotide from the 3′ end of the gRNA, and the fourth nucleotide from the 3′ end of the gRNA each comprise a phosphorothioate linkage.

In some embodiments, the gRNA comprises a thioPACE linkage. In some embodiments, the gRNA comprises a backbone in which one or more non-bridging oxygen atoms have been replaced with a sulfur atom and one or more non-bridging oxygen atoms have been replaced with an acetate group. In some embodiments, the nucleotide at the 5′ end of the gRNA, the second nucleotide from the 5′ end of the gRNA, and the third nucleotide from the 5′ end of the gRNA each comprise a thioPACE linkage. In some embodiments, the nucleotide at the 3′ end of the gRNA, the second nucleotide from the 3′ end of the gRNA, and the third nucleotide from the 3′ end of the gRNA each comprise a thioPACE linkage. In some embodiments, the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA each comprise a thioPACE linkage. In some embodiments, the second nucleotide from the 3′ end of the gRNA, the third nucleotide from the 3′ end of the gRNA, and at the fourth nucleotide from the 3′ end of the gRNA each comprise a thioPACE linkage. In some embodiments, the nucleotide at the 5′ end of the gRNA, the second nucleotide from the 5′ end of the gRNA, the third nucleotide from the 5′ end, the second nucleotide from the 3′ end of the gRNA, the third nucleotide from the 3′ end of the gRNA, and the fourth nucleotide from the 3′ end of the gRNA each comprise a thioPACE linkage.

Some exemplary, non-limiting embodiments of modifications, e.g., chemical modifications, suitable for use in connection with the guide RNAs and genetic engineering methods provided herein have been described above. Additional suitable modifications, e.g., chemical modifications, will be apparent to those of skill in the art based on the present disclosure and the knowledge in the art, including, but not limited to those described in Hendel, A. et al., Nature Biotech., 2015, Vol 33, No. 9; in WO/2017/214460; in WO/2016/089433; and/or in WO/2016/164356; each one of which is herein incorporated by reference in its entirety.

gRNAs Targeting CD33

The present disclosure provides a number of useful gRNAs that can target an endonuclease to human CD33. Table 1 below illustrates target domains in human endogenous CD33 that can be bound by gRNAs described herein.

TABLE 1
Target domains of human CD33 bound by various gRNAs described
herein. For each target domain, the first sequence represents the
sequence corresponding to the targeting domain sequence of the gRNA,
and the second sequence is the reverse complement thereof.

| gRNA Name | Target Domain Sequences |
|---|---|
| gRNA A | CCCCAGGACTACTCACTCCT (SEQ ID NO: 1) |
| | AGGAGTGAGTAGTCCTGGGG (SEQ ID NO: 5) |
| gRNA B | ACCGAGGAGTGAGTAGTCCT (SEQ ID NO: 2) |
| | AGGACTACTCACTCCTCGGT (SEQ ID NO: 6) |
| gRNA C | GGTGGGGGCAGCTGACAACC (SEQ ID NO: 3) |
| | GGTTGTCAGCTGCCCCCACC (SEQ ID NO: 7) |
| gRNA D | CGGTGCTCATAATCACCCCA (SEQ ID NO: 4) |
| | TGGGGTGATTATGAGCACCG (SEQ ID NO: 8) |

TABLE 2
Targeting domains of gRNAs complementary to human CD33. For each
gRNA, the first sequence represents the DNA equivalent including
thymine, and the second sequence represents an RNA equivalent
that includes uracil in place of thymine.

| gRNA Name | gRNA Sequence | PAM |
|---|---|---|
| gRNA A | CCCCAGGACTACTCACTCCT (SEQ ID NO: 1) | |
| | CCCCAGGACUACUCACUCCU (SEQ ID NO: 9) | CGG |
| gRNA B | ACCGAGGAGTGAGTAGTCCT (SEQ ID NO: 2) | |
| | ACCGAGGAGUGAGUAGUCCU (SEQ ID NO: 10) | GGG |
| gRNA C | GGTGGGGGCAGCTGACAACC (SEQ ID NO: 3) | |
| | GGUGGGGGCAGCUGACAACC (SEQ ID NO: 11) | AGG |
| gRNA D | CGGTGCTCATAATCACCCCA (SEQ ID NO: 4) | |
| | CGGUGCUCAUAAUCACCCCA (SEQ ID NO: 12) | CGG |

The CD33 (CCDS33084.1) cDNA sequence is provided below as SEQ ID NO: 13. Exon 3 is underlined.

(SEQ ID NO: 13)
ATGCCGCTGCTGCTACTGCTGCCCCTGCTGTGGGCAGGGGCCCTGGCTA
TGGATCCAAATTTCTGGCTGCAAGTGCAGGAGTCAGTGACGGTACAGGA
GGGTTTGTGCGTCCTCGTGCCCTGCACTTTCTTCCATCCCATACCCTAC
TACGACAAGAACTCCCCAGTTCATGGTTACTGGTTCCGGGAAGGAGCCA
TTATATCCAGGGACTCTCCAGTGGCCACAAACAAGCTAGATCAAGAAGT
ACAGGAGGAGACTCAGGGCAGATTCCGCCTCCTTGGGGATCCCAGTAGG
AACAACTGCTCCCTGAGCATCGTAGACGCCAGGAGGAGGGATAATGGTT
CATACTTCTTTCGGATGGAGAGAGGAAGTACCAAATACAGTTACAAATC
TCCCCAGCTCTCTGTGCATGTGACAGACTTGACCCACAGGCCCAAAATC
CTCATCCCTGGCACTCTAGAACCCGGCCACTCCAAAAACCTGACCTGCT
CTGTGTCCTGGGCCTGTGAGCAGGGAACACCCCCGATCTTCTCCTGGTT
GTCAGCTGCCCCCACCTCCCTGGGCCCCAGGACTACTCACTCCTCGGTG
CTCATAATCACCCCACGGCCCCAGGACCACGGCACCAACCTGACCTGTC
AGGTGAAGTTCGCTGGAGCTGGTGTGACTACGGAGAGAACCATCCAGCT
CAACGTCACCTATGTTCCACAGAACCCAACAACTGGTATCTTTCCAGGA
GATGGCTCAGGGAAACAAGAGACCAGAGCAGGAGTGGTTCATGGGGCCA
TTGGAGGAGCTGGTGTTACAGCCCTGCTCGCTCTTTGTCTCTGCCTCAT
CTTCTTCATAGTGAAGACCCACAGGAGGAAAGCAGCCAGGACAGCAGTG
GGCAGGAATGACACCCACCCTACCACAGGGTCAGCCTCCCCGAAACACC
AGAAGAAGTCCAAGTTACATGGCCCCACTGAAACCTCAAGCTGTTCAGG
TGCCGCCCCTACTGTGGAGATGGATGAGGAGCTGCATTATGCTTCCCTC
AACTTTCATGGGATGAATCCTTCCAAGGACACCTCCACCGAATACTCAG
AGGTCAGGACCCAGTGA

Exon 3 of CD33 is provided separately below as SEQ ID NO: 14. Underlining indicates the regions complementary to gRNA A, gRNA B, gRNA C, gRNA D (or the reverse complement thereof). Note that the target regions for gRNA A, gRNA B, and gRNA D partially overlap.

(SEQ ID NO: 14)
ACTTGACCCACAGGCCCAAAATCCTCATCCCTGGCACTCTAGAACCCGG
CCACTCCAAAAACCTGACCTGCTCTGTGTCCTGGGCCTGTGAGCAGGGA
ACACCCCCGATCTTCTCCTGGTTGTCAGCTGCCCCCACCTCCCTGGGCC
CCAGGACTACTCACTCCTCGGTGCTCATAATCACCCCACGGCCCCAGGA
CCACGGCACCAACCTGACCTGTCAGGTGAAGTTCGCTGGAGCTGGTGTG
ACTACGGAGAGAACCATCCAGCTCAACGTCACCT

Dual gRNA Compositions and Uses Thereof

In some embodiments, a gRNA described herein (e.g., a gRNA of Table 2) can be used in combination with a second gRNA, e.g., for directing nucleases to two sites in a genome. For instance, in some embodiments it is desired to produce a hematopoietic cell that is deficient for CD33 and a second lineage-specific cell surface antigen, e.g., so that the cell can be resistant to two agents: an anti-CD33 agent and an agent targeting the second lineage-specific cell surface antigen. In some embodiments, it is desirable to contact a cell with two different gRNAs that target different regions of CD33, in order to make two cuts and create a deletion between the two cut sites. Accordingly, the disclosure provides various combinations of gRNAs.

In some embodiments, two or more (e.g., 3, 4, or more) gRNAs described herein are admixed. In some embodiments, each gRNA is in a separate container. In some embodiments, a kit described herein (e.g., a kit comprising one or more gRNAs according to Table 2) also comprises a Cas9 molecule, or a nucleic acid encoding the Cas9 molecule.

In some embodiments, the first and second gRNAs are gRNAs according to Table 2 or variants thereof.

In some embodiments, the first gRNA is a CD33 gRNA described herein (e.g., a gRNA of Table 2 or a variant thereof) and the second gRNA targets a lineage-specific cell-surface antigen chosen from: BCMA, CD19, CD20, CD30, ROR1, B7H6, B7H3, CD23, CD38, C-type lectin like molecule-1, CS1, IL-5, L1-CAM, PSCA, PSMA, CD138, CD133, CD70, CD7, CD13, NKG2D, NKG2D ligand, CLEC12A, CD11, CD123, CD56, CD34, CD14, CD66b, CD41, CD61, CD62, CD235a, CD146, CD326, LMP2, CD22, CD52, CD10, CD3/TCR, CD79/BCR, and CD26.

In some embodiments, the first gRNA is a CD33 gRNA described herein (e.g., a gRNA according to Table 2 or a variant thereof) and the second gRNA targets a lineage-specific cell-surface antigen associated with a specific type of cancer, such as, without limitation, CD20, CD22 (Non-Hodgkin's lymphoma, B-cell lymphoma, chronic lymphocytic leukemia (CLL)), CD52 (B-cell CLL), CD33 (Acute myelogenous leukemia (AML)), CD10 (gp100) (Common (pre-B) acute lymphocytic leukemia and malignant melanoma), CD3/T-cell receptor (TCR) (T-cell lymphoma and leukemia), CD79/B-cell receptor (BCR) (B-cell lymphoma and leukemia), CD26 (epithelial and lymphoid malignancies), human leukocyte antigen (HLA)-DR, HLA-DP, and HLA-DQ (lymphoid malignancies), RCAS1 (gynecological carcinomas, biliary adenocarcinomas and ductal adenocarcinomas of the pancreas) as well as prostate specific membrane antigen.

In some embodiments, the first gRNA is a CD33 gRNA described herein (e.g., a gRNA according to Table 2 or a variant thereof) and the second gRNA targets a lineage-specific cell-surface antigen chosen from: CD7, CD13, CD19, CD22, CD20, CD25, CD32, CD38, CD44, CD45, CD47, CD56, 96, CD117, CD123, CD135, CD174, CLL-1, folate receptor β, IL1RAP, MUC1, NKG2D/NKG2DL, TIM-3, or WT1.

In some embodiments, the first gRNA is a CD33 gRNA described herein (e.g., a gRNA according to Table 2 or a variant thereof) and the second gRNA targets a lineage-specific cell-surface antigen chosen from: CD1a, CD1b, CD1c, CD1d, CD1e, CD2, CD3, CD3d, CD3e, CD3g, CD4, CD5, CD6, CD7, CD8a, CD8b, CD9, CD10, CD11a, CD11b, CD11c, CD11d, CDw12, CD13, CD14, CD15, CD16, CD16b, CD17, CD18, CD19, CD20, CD21, CD22, CD23, CD24, CD25, CD26, CD27, CD28, CD29, CD30, CD31, CD32a, CD32b, CD32c, CD34, CD35, CD36, CD37, CD38, CD39, CD40, CD41, CD42a, CD42b, CD42c, CD42d, CD43, CD44, CD45, CD45RA, CD45RB, CD45RC, CD45RO, CD46, CD47, CD48, CD49a, CD49b, CD49c, CD49d, CD49e, CD49f, CD50, CD51, CD52, CD53, CD54, CD55, CD56, CD57, CD58, CD59, CD60a, CD61, CD62E, CD62L, CD62P, CD63, CD64a, CD65, CD65s, CD66a, CD66b, CD66c, CD66F, CD68, CD69, CD70, CD71, CD72, CD73, CD74, CD75, CD75S, CD77, CD79a, CD79b, CD80, CD81, CD82, CD83, CD84, CD85A, CD85C, CD85D, CD85E, CD85F, CD85G, CD85H, CD85I, CD85J, CD85K, CD86, CD87, CD88, CD89, CD90, CD91, CD92, CD93, CD94, CD95, CD96, CD97, CD98, CD99, CD99R, CD100, CD101, CD102, CD103, CD104, CD105, CD106, CD107a, CD107b, CD108, CD109, CD110, CD111, CD112, CD113, CD114, CD115, CD116, CD117, CD118, CD119, CD120a, CD120b, CD121a, CD121b, CD121a, CD121b, CD122, CD123, CD124, CD125, CD126, CD127, CD129, CD130, CD131, CD132, CD133, CD134, CD135, CD136, CD137, CD138, CD139, CD140a, CD140b, CD141, CD142, CD143, CD14, CDw145, CD146, CD147, CD148, CD150, CD152, CD152, CD153, CD154, CD155, CD156a, CD156b, CD156c, CD157, CD158b1, CD158b2, CD158d, CD158e1/e2, CD158f, CD158g, CD158 h, CD158i, CD158j, CD158k, CD159a, CD159c, CD160, CD161, CD163, CD164, CD165, CD166, CD167a, CD168, CD169, CD170, CD171, CD172a, CD172b, CD172g, CD173, CD174, CD175, CD175s, CD176, CD177, CD178, CD179a, CD179b, CD180, CD181, CD182, CD183, CD184, CD185, CD186, CD191, CD192, CD193, CD194, CD195, CD196, CD197, CDw198, CDw199, CD200, CD201, CD202b, CD203c, CD204, CD205, CD206, CD207, CD208, CD209, CD210a, CDw210b, CD212, CD213a1, CD213a2, CD215, CD217, CD218a, CD218b, CD220, CD221, CD222, CD223, CD224, CD225, CD226, CD227, CD228, CD229, CD230, CD231, CD232, CD233, CD234, CD235a, CD235b, CD236, CD236R, CD238, CD239, CD240, CD241, CD242, CD243, CD244, CD245, CD246, CD247, CD248, CD249, CD252, CD253, CD254, CD256, CD257, CD258, CD261, CD262, CD263, CD264, CD265, CD266, CD267, CD268, CD269, CD270, CD272, CD272, CD273, CD274, CD275, CD276, CD277, CD278, CD279, CD280, CD281, CD282, CD283, CD284, CD286, CD288, CD289, CD290, CD292, CDw293, CD294, CD295, CD296, CD297, CD298, CD299, CD300a, CD300c, CD300e, CD301, CD302, CD303, CD304, CD305, CD306, CD307a, CD307b, CD307c, CD307d, CD307e, CD309, CD312, CD314, CD315, CD316, CD317, CD318, CD319, CD320, CD321, CD322, CD324, CD325, CD326, CD327, CD328, CD329, CD331, CD332, CD333, CD334, CD335, CD336, CD337, CD338, CD339, CD340, CD344, CD349, CD350, CD351, CD352, CD353, CD354, CD355, CD357, CD358, CD359, CD360, CD361, CD362 or CD363.

In some embodiments, the first gRNA is a CD33 gRNA described herein (e.g., a gRNA according to Table 2 or a variant thereof) and the second gRNA targets a lineage-specific cell-surface antigen chosen from: CD19; CD123; CD22; CD30; CD171; CS-1 (also referred to as CD2 subset 1, CRACC, SLAMF7, CD319, and 19A24); C-type lectin-like molecule-1 (CLECL1); epidermal growth factor receptor variant III (EGFRvIII); ganglioside G2 (CD2); ganglioside GD3 (aNeu5Ac(2-8)aNeu5Ac(2-3)bDGalp(1-4)bDGlep(1-1)Cer); TNF receptor family member B cell maturation (BCMA), Tn antigen ((Tn Ag) or (GalNAc.alpha.-Ser/Thr)); prostate-specific membrane antigen (PSMA); Receptor tyrosine kinase-like orphan receptor 1 (ROR1); Fms-Like tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor I receptor (IGF-I receptor), carbonic anhydrase IX (CAIX), Proteasome (Prosome, Macropain) Subunit, Beta Type 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(1-4)bDG1cp(1-1)Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRCSD); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex; locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1), ETS translocation-variant gene 6, located on chromosome 12p (ETV6-AML); sperm protein 17 (SPA17); X Antigen Family, member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCTA-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-1AP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxy esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2), lymphocyte antigen 75 (LY75); Glypican-3 (GPC3); Fc receptor-like 5 (FCRL5); and immunoglobulin lambda-like polypeptide 1 (IGLL1).

In some embodiments, the first gRNA is a CD33 gRNA described herein (e.g., a gRNA according to Table 2 or a variant thereof) and the second gRNA targets a lineage-specific cell-surface antigen chosen from: CD11a, CD18, CD19, CD20, CD31, CD34, CD44, CD45, CD47, CD51, CD58, CD59, CD63, CD97, CD99, CD100, CD102, CD123, CD127, CD133, CD135, CD157, CD172b, CD217, CD300a, CD305, CD317, CD321, and CLL1.

In some embodiments, the first gRNA is a CD33 gRNA described herein (e.g., a gRNA according to Table 2 or a variant thereof) and the second gRNA targets a lineage-specific cell-surface antigen chosen from: CD123, CLL1, CD38, CD135 (FLT3), CD56 (NCAM1), CD117 (c-KIT), FRβ (FOLR2), CD47, CD82, TNFRSF1B (CD120B), CD191, CD96, PTPRJ (CD148), CD70, LILRB2 (CD85D), CD25 (IL2Ralpha), CD44, CD96, NKG2D Ligand, CD45, CD7, CD15, CD19, CD20, CD22, CD37, and CD82.

In some embodiments, the first gRNA is a CD33 gRNA described herein (e.g., a gRNA according to Table 2 or a variant thereof) and the second gRNA targets a lineage-specific cell-surface antigen chosen from: CD7, CD11a, CD15, CD18, CD19, CD20, CD22, CD25, CD31, CD34, CD37, CD38, CD44, CD45, CD47, CD51, CD56, CD58, CD59, CD63, CD70, CD82, CD85D, CD96, CD97, CD99, CD100, CD102, CD117, CD120B, CD123, CD127, CD133, CD135, CD148, CD157, CD172b, CD191, CD217, CD300a, CD305, CD317, CD321, CLL1, FRβ (FOLR2), or NKG2D Ligand.

In some embodiments, the first gRNA is a CD33 gRNA described herein (e.g., a gRNA according to Table 2 or a variant thereof) and the second gRNA targets CLL-1. In some embodiments, the first gRNA is a CD33 gRNA described herein (e.g., a gRNA according to Table 2 or a variant thereof) and the second gRNA targets CD123.

In some embodiments, the first gRNA is a CD33 gRNA described herein (e.g., a gRNA according to Table 2 or a variant thereof) and the second gRNA comprises a sequence from Table A. In some embodiments, the first gRNA is a CD33 gRNA comprising a targeting domain, wherein the targeting domain comprises a sequence of SEQ ID NO: 9, and the second gRNA comprises a targeting domain corresponding to a sequence of Table A. In some embodiments, the first gRNA is a CD33 gRNA comprising a targeting domain, wherein the targeting domain comprises a sequence of SEQ ID NO: 10, and the second gRNA comprises a targeting domain corresponding to a sequence of Table A. In some embodiments, the first gRNA is a CD33 gRNA comprising a targeting domain, wherein the targeting domain comprises a sequence of SEQ ID NO: 11, and the second gRNA comprises a targeting domain corresponding to a sequence of Table A. In some embodiments, the first gRNA is a CD33 gRNA comprising a targeting domain, wherein the targeting domain comprises a sequence of SEQ ID NO: 12, and the second gRNA comprises a targeting domain corresponding to a sequence of Table A. In some embodiments, the second gRNA is a gRNA disclosed in any of WO2017/066760, WO2019/046285, WO/2018/160768, or Borot et al. PNAS Jun. 11, 2019 116 (24) 11978-11987, each of which is incorporated herein by reference in its entirety.

TABLE A
Exemplary gRNA spacer sequences. Certain gRNA spacer sequences are
followed by a PAM sequence, indicated by a space in the text.

| gRNA target | gRNA spacer sequence | SEQ ID NO: |
|---|---|---|
| hCD33 | ACCTGTCAGGTGAAGTTCGC TGG | 26 |
| hCD33 | TGGCCGGGTTCTAGAGTGCC AGG | 27 |
| hCD33 | GGCCGGGTTCTAGAGTGCCA GGG | 28 |
| hCD33 | CACCGAGGAGTGAGTAGTCC TGG | 29 |
| hCD33 | TCCAGCGAACTTCACCTGAC AGG | 30 |
| CD33 (in intron 1) | GCTGTGGGGAGAGGGGTTGT | 31 |
| CD33 (in intron 1) | CTGTGGGGAGAGGGGTTGTC | 32 |
| CD33 (in intron 1) | TGGGGAAACGAGGGTCAGCT | 33 |
| CD33 (in intron 1) | GGGCCCCTGTGGGGAAACGA | 34 |
| CD33 (in intron 1) | AGGGCCCCTGTGGGGAAACG | 35 |
| CD33 (in intron 1) | GCTGACCCTCGTTTCCCCAC | 36 |
| CD33 (in intron 1) | CTGACCCTCGTTTCCCCACA | 37 |
| CD33 (in intron 1) | TGACCCTCGTTTCCCCACAG | 38 |
| CD33 (in intron 1) | CCATAGCCAGGGCCCCTGTG | 39 |
| CD33 (in intron 2) | GCATGTGACAGGTGAGGCAC | 40 |
| CD33 (in intron 2) | TGAGGCACAGGCTTCAGAAG | 41 |
| CD33 (in intron 2) | AGGCTTCAGAAGTGGCCGCA | 42 |
| CD33 (in intron 2) | GGCTTCAGAAGTGGCCGCAA | 43 |
| CD33 (in intron 2) | GTACCCATGAACTTCCCTTG | 44 |
| CD33 (in intron 2) | GTGGCCGCAAGGGAAGTTCA | 45 |
| CD33 (in intron 2) | TGGCCGCAAGGGAAGTTCAT | 46 |
| CD33 (in intron 2) | GGAAGTTCATGGGTACTGCA | 47 |
| CD33 (in intron 2) | TTCATGGGTACTGCAGGGCA | 48 |
| CD33 (in intron 2) | CTAAACCCCTCCCAGTACCA | 49 |
| CD33 (in intron 1) | CACTCACCTGCCCACAGCAG | 50 |
| CD33 (in intron 1) | CCCTGCTGTGGGCAGGTGAG | 51 |
| CD33 (in intron 1) | TGGGCAGGTGAGTGGCTGTG | 52 |
| CD33 (in intron 1) | GGTGAGTGGCTGTGGGGAGA | 53 |
| CD33 (in intron 1) | GTGAGTGGCTGTGGGGAGAG | 54 |
| CD33 (exon 2) | ATCCATAGCCAGGGCCCCTG | 55 |
| CD33 (exon 2) | TCCATAGCCAGGGCCCCTGT | 56 |
| CD33 (exon 2) | CCATAGCCAGGGCCCCTGTG | 39 |
| CD33 (exon 2) | TCGTTTCCCCACAGGGGCCC | 57 |
| CD33 (exon 2) | TGGCTATGGATCCAAATTTC | 58 |
| CD33 (exon 2) | TGGGGAAACGAGGGTCAGCT | 33 |
| CD33 (exon 2) | GGGCCCCTGTGGGGAAACGA | 34 |
| CD33 (exon 2) | AGAAATTTGGATCCATAGCC AGG | 59 |
| CD33 (exon 3) | ATCCCTGGCACTCTAGAACC CGG | 60 |
| CD33 (exon 3) | CCTCACTAGACTTGACCCAC AGG | 61 |

### Cells Comprising Two Mutations

In some embodiments, an engineered cell described herein comprises two mutations, the first mutation being in CD33 and the second mutation being in a second lineage-specific cell surface antigen. Such a cell can, in some embodiments, be resistant to two agents: an anti-CD33 agent and an agent targeting the second lineage-specific cell surface antigen. In some embodiments, such a cell can be produced using two or more gRNAs described herein, e.g., a gRNA of Table 2 and a second gRNA. In some embodiments, the cell can be produced using, e.g., a ZFN or TALEN. The disclosure also provides populations comprising cells described herein.

In some embodiments, the second mutation is at a gene encoding a lineage-specific cell-surface antigen, e.g., one listed in the preceding section. In some embodiments, the second mutation is at a site listed in Table A.

Typically, a mutation effected by the methods and compositions provided herein, e.g., a mutation in a target gene, such as, for example, CD33 and/or any other target gene mentioned in this disclosure, results in a loss of function of a gene product encoded by the target gene, e.g., in the case of a mutation in the CD33 gene, in a loss of function of a CD33 protein. In some embodiments, the loss of function is a reduction in the level of expression of the gene product, e.g., reduction to a lower level of expression, or a complete abolishment of expression of the gene product. In some embodiments, the mutation results in the expression of a non-functional variant of the gene product. For example, in the case of the mutation generating a premature stop codon in the encoding sequence, a truncated gene product, or, in the case of the mutation generating a nonsense or mis sense mutation, a gene product characterized by an altered amino acid sequence, which renders the gene product non-functional. In some embodiments, the function of a gene product is binding or recognition of a binding partner. In some embodiments, the reduction in expression of the gene product, e.g., of CD33, of the second lineage-specific cell-surface antigen, or both, is to less than or equal to 50%, less than or equal to 40%, less than or equal to 30%, less than or equal to 20%, less than or equal to 10%, less than or equal to 5%, less than or equal to 2%, or less than or equal to 1% of the level in a wild-type or non-engineered counterpart cell.

In some embodiments, at least 40%, at least 50%, at least 60%, at least 70%, at least 75%, at least 80%, at least 85%, at least 90%, or at least 95% of copies of CD33 in the population of cells generated by the methods and/or using the compositions provided herein have a mutation. In some embodiments, at least 40%, at least 50%, at least 60%, at least 70%, at least 75%, at least 80%, at least 85%, at least 90%, or at least 95% of copies of the second lineage-specific cell surface antigen in the population of cells have a mutation. In some embodiments, at least 40%, at least 50%, at least 60%, at least 70%, at least 75%, at least 80%, at least 85%, at least 90%, or at least 95% of copies of CD33 and of the second lineage-specific cell surface antigen in the population of cells have a mutation. In some embodiments, the population comprises one or more wild-type cells. In some embodiments, the population comprises one or more cells that comprise one wild-type copy of CD33. In some embodiments, the population comprises one or more cells that comprise one wild-type copy of the second lineage-specific cell surface antigen.

### Cells

In some embodiments, a cell (e.g., an HSC or HPC) having a modification of CD33 is made using a nuclease and/or a gRNA described herein. In some embodiments, a cell (e.g., HSC or HPC) having a modification of CD33 and a modification of a second lineage-specific cell surface antigen is made using a nuclease and/or a gRNA described herein. It is understood that the cell can be made by contacting the cell itself with the nuclease and/or a gRNA, or the cell can be the daughter cell of a cell that was contacted with the nuclease and/or gRNA. In some embodiments, a cell described herein (e.g., an HSC) is capable of reconstituting the hematopoietic system of a subject. In some embodiments, a cell described herein (e.g., an HSC) is capable of one or more of (e.g., all of): engrafting in a human subject, producing myeloid lineage cells, and producing and lymphoid lineage cells.

In some embodiments, the cell comprises only one genetic modification. In some embodiments, the cell is only genetically modified at the CD33 locus. In some embodiments, the cell is genetically modified at a second locus. In some embodiments, the cell does not comprise a transgenic protein, e.g., does not comprise a CAR.

In some embodiments, a modified cell described herein comprises substantially no CD33 protein. In some embodiments, a modified cell described herein comprises substantially no wild-type CD33 protein, but comprises mutant CD33 protein. In some embodiments, the mutant CD33 protein is not bound by an agent that targets CD33 for therapeutic purposes.

In some embodiments, the cells are hematopoietic cells, e.g., hematopoietic stem cells. Hematopoietic stem cells (HSCs) are typically capable of giving rise to both myeloid and lymphoid progenitor cells that further give rise to myeloid cells (e.g., monocytes, macrophages, neutrophils, basophils, dendritic cells, erythrocytes, platelets, etc) and lymphoid cells (e.g., T cells, B cells, NK cells), respectively. HSCs are characterized by the expression of the cell surface marker CD34 (e.g., CD34+), which can be used for the identification and/or isolation of HSCs, and absence of cell surface markers associated with commitment to a cell lineage.

In some embodiments, a population of cells described herein comprises a plurality of hematopoietic stem cells; in some embodiments, a population of cells described herein comprises a plurality of hematopoietic progenitor cells; and in some embodiments, a population of cells described herein comprises a plurality of hematopoietic stem cells and a plurality of hematopoietic progenitor cells.

In some embodiments, the HSCs are obtained from a subject, such as a human subject. Methods of obtaining HSCs are described, e.g., in PCT/US2016/057339, which is herein incorporated by reference in its entirety. In some embodiments, the HSCs are peripheral blood HSCs. In some embodiments, the mammalian subject is a non-human primate, a rodent (e.g., mouse or rat), a bovine, a porcine, an equine, or a domestic animal. In some embodiments, the HSCs are obtained from a human subject, such as a human subject having a hematopoietic malignancy. In some embodiments, the HSCs are obtained from a healthy donor. In some embodiments, the HSCs are obtained from the subject to whom the immune cells expressing the chimeric receptors will be subsequently administered. HSCs that are administered to the same subject from which the cells were obtained are referred to as autologous cells, whereas HSCs that are obtained from a subject who is not the subject to whom the cells will be administered are referred to as allogeneic cells.

In some embodiments, at least 40%, at least 50%, at least 60%, at least 70%, at least 75%, at least 80%, at least 85%, at least 90%, or at least 95% of copies of CD33 in the population of cells have a mutation. By way of example, a population can comprise a plurality of different CD33 mutations and each mutation of the plurality contributes to the percent of copies of CD33 in the population of cells that have a mutation.

In some embodiments, the expression of CD33 on the genetically engineered hematopoietic cell is compared to the expression of CD33 on a naturally occurring hematopoietic cell (e.g., a wild-type counterpart). In some embodiments, the genetic engineering results in a reduction in the expression level of CD33 by at least 50%, at least 60%, at least 70%, at least 80%, at least 90%, at least 91%, at least 92%, at least 93%, at least 94%, at least 95%, at least 96%, at least 97%, at least 98%, or at least 99% as compared to the expression of CD33 on a naturally occurring hematopoietic cell (e.g., a wild-type counterpart). For example, in some embodiments, the genetically engineered hematopoietic cell expresses less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, or less than 1% of CD33 as compared to a naturally occurring hematopoietic cell (e.g., a wild-type counterpart).

In some embodiments, the genetic engineering results in a reduction in the expression level of wild-type CD33 by at least 50%, at least 60%, at least 70%, at least 80%, at least 90%, at least 91%, at least 92%, at least 93%, at least 94%, at least 95%, at least 96%, at least 97%, at least 98%, or at least 99% as compared to the expression of the level of wild-type CD33 on a naturally occurring hematopoietic cell. That is, in some embodiments, the genetically engineered hematopoietic cell expresses less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, or less than 1% of CD33 as compared to a naturally occurring hematopoietic cell (e.g., a wild-type counterpart).

In some embodiments, the genetic engineering results in a reduction in the expression level of wild-type lineage-specific cell surface antigen (e.g., CD33) by at least 50%, at least 60%, at least 70%, at least 80%, at least 90%, at least 91%, at least 92%, at least 93%, at least 94%, at least 95%, at least 96%, at least 97%, at least 98%, or at least 99% as compared to a suitable control (e.g., a cell or plurality of cells). In some embodiments, the suitable control comprises the level of the wild-type lineage-specific cell surface antigen measured or expected in a plurality of non-engineered cells from the same subject. In some embodiments, the suitable control comprises the level of the wild-type lineage-specific cell surface antigen measured or expected in a plurality of cells from a healthy subject. In some embodiments, the suitable control comprises the level of the wild-type lineage-specific cell surface antigen measured or expected in a population of cells from a pool of healthy individuals (e.g., 10, 20, 50, or 100 individuals). In some embodiments, the suitable control comprises the level of the wild-type lineage-specific cell surface antigen measured or expected in a subject in need of a treatment described herein, e.g., an anti-CD33 therapy, e.g., wherein the subject has a cancer, wherein cells of the cancer express CD33

In some embodiments, a method of making described herein comprises a step of providing a wild-type cell, e.g., a wild-type hematopoietic stem or progenitor cell. In some embodiments, the wile-type cell is an un-edited cell comprising (e.g., expressing) two functional copies of the lineage-specific cell surface antigen (e.g., CD33, CD123, and/or CLL1). In some embodiments, the cell comprises a CD33 gene sequence according to SEQ ID NO: 13. In some embodiments, the cell comprises a CD33 gene sequence encoding a CD33 protein that is encoded in SEQ ID NO: 13, e.g., the CD33 gene sequence may comprise one or more silent mutations relative to SEQ ID NO: 13. In some embodiments, the cell used in the method is a naturally occurring cell or a non-engineered cell. In some embodiments, the wild-type cell expresses the lineage-specific cell surface antigen (e.g., CD33), or gives rise to a more differentiated cell that expresses the lineage-specific cell surface antigen at a level comparable to (or within 90%-110%, 80%-120%, 70%-130%, 60-140%, or 50%-150% of) HL60 or MOLM-13 cells. In some embodiments, the wild-type cell binds an antibody that binds the lineage-specific cell surface antigen (e.g., an anti-CD33 antibody, e.g., P67.6), or gives rise to a more differentiated cell that binds the antibody at a level comparable to (or within 90%-110%, 80%-120%, 70%-130%, 60-140%, or 50%-150% of) binding of the antibody to HL60 or MOLM-13 cells. Antibody binding may be measured, for example, by flow cytometry, e.g., as described in Example 4.

### Methods of Treatment and Administration

In some embodiments, an effective number of CD33-modified cells described herein is administered in combination with an anti-CD33 therapy, e.g., an anti-CD33 cancer therapy. In some embodiments, an effective number of cells comprising a modified CD33 and a modified second lineage-specific cell surface antigen are administered in combination with an anti-CD33 therapy, e.g., an anti-CD33 cancer therapy. In some embodiments, the anti-CD33 therapy comprises an antibody, an ADC, or an immune cell expressing a CAR.

It is understood that when agents (e.g., CD33-modified cells and an anti-CD33 therapy) are administered in combination, the agent may be administered at the same time or at different times in temporal proximity. Furthermore, the agents may be admixed or in separate volumes. For example, in some embodiments, administration in combination includes administration in the same course of treatment, e.g., in the course of treating a cancer with an anti-CD33 therapy, the subject may be administered an effective number of CD33-modified cells concurrently or sequentially, e.g., before, during, or after the treatment, with the anti-CD33 therapy.

In some embodiments, the agent that targets a CD33 as described herein is an immune cell that expresses a chimeric receptor, which comprises an antigen-binding fragment (e.g., a single-chain antibody) capable of binding to CD33. The immune cell may be, e.g., a T cell (e.g., a CD4+ or CD8+ T cell) or an NK cell.

A Chimeric Antigen Receptor (CAR) can comprise a recombinant polypeptide comprising at least an extracellular antigen binding domain, a transmembrane domain and a cytoplasmic signaling domain comprising a functional signaling domain, e.g., one derived from a stimulatory molecule. In one some embodiments, the cytoplasmic signaling domain further comprises one or more functional signaling domains derived from at least one costimulatory molecule, such as 4-1BB (i.e., CD137), CD27 and/or CD28 or fragments of those molecules. The extracellular antigen binding domain of the CAR may comprise a CD33-binding antibody fragment. The antibody fragment can comprise one or more CDRs, the variable region (or portions thereof), the constant region (or portions thereof), or combinations of any of the foregoing.

Amino acid and nucleic acid sequences of an exemplary heavy chain variable region and light chain variable region of an anti-human CD33 antibody are provided below. The CDR sequences are shown in boldface and underlined in the amino acid sequences.

Amino acid sequence of anti-CD33 Heavy Chain
Variable Region
(SEQ ID NO: 15)
QVQLQQPGAEVVKPGASVKMSCKASGYTFTSYYIHWIKQTPGQGLEWVG
VIYPGNDDISYNQKFQGKATLTADKSSTTAYMQLSSLTSEDSAVYYCAR
EVRLRYFDVWGQGTTVTVSS
Amino acid sequence of anti-CD33 Light Chain
Variable Region
(SEQ ID NO: 16)
EIVLIQSPGSLAVSPGERVIMSCKSSQSVFFSSSQKNYLAWYQQIPGQS
PRLLIYWASTRESGVPDRFTGSGSGTDFTLTISSVQPEDLAIYYCHQYL
SSRTFGQGTKLEIKR

The anti-CD33 antibody binding fragment for use in constructing the agent that targets CD33 as described herein may comprise the same heavy chain and/or light chain CDR regions as those in SEQ ID NO:15 and SEQ ID NO:16. Such antibodies may comprise amino acid residue variations in one or more of the framework regions. In some instances, the anti-CD33 antibody fragment may comprise a heavy chain variable region that shares at least 70% sequence identity (e.g., 75%, 80%, 85%, 90%, 95%, or higher) with SEQ ID NO:15 and/or may comprise a light chain variable region that shares at least 70% sequence identity (e.g., 75%, 80%, 85%, 90%, 95%, or higher) with SEQ ID NO:16.
Exemplary chimeric receptor component sequences are provided in Table 3 below.

TABLE 3
Exemplary components of a chimeric receptor

| Chimeric receptor component | Amino acid sequence |
|---|---|
| Antigen-binding fragment | Light chain-GSTSSGSGKPGSGEGSTKG |
| | (SEQ ID NO: 17)-Heavy chain |
| CD28 costimulatory domain | IEVMYPPPYLDNEKSNGTIIHVKGKHLCPSP |
| | LFPGPSKPFWVLVVVGGVLACYSLLVTVA |
| | FIIFWVRSKRSRLLHSDYMNMTPRRPGPTR |
| | KHYQPYAPPRDFAAYRS (SEQ ID NO: 18) |
| ICOS costimulatory domain (boldface), | LSIFDPPPFKVTLTGGYLHIYESQLCCQLKF |
| ICOS transmembrane domain (italics) | WLPIGCAAFVVVCILGCILICWLTKKKYSSS |
| and a portion of the extracellular | VHDPNGEYMFMRAVNTAKKSRLTDVTL |
| domain of ICOS (underlined) | (SEQ ID NO: 19) |
| ICOS costimulatory domain | CWLTKKKYSSSVHDPNGEYMFMRAVNTA |
| | KKSRLTDVTL (SEQ ID NO: 20) |
| CD28/ICOS chimera (the ICOS portion | IEVMYPPPYLDNEKSNGTIIHVKGKHLCPSPL |
| shown in underline) including the hinge | FPGPSKPFWVLVVVGGVLACYSLLVTVA |
| domain (italics) and transmembrane | FIIFWVRSKRSRLLHSDYMFMRAVNTAKK |
| domain (bold) from CD28 | SRLTDVTL (SEQ ID NO: 21) |
| CD3ζ cytoplasmic signaling domain | RVKFSRSADAPAYQQGQNQLYNELNLGRR |
| | EEYDVLDKRRGRDPEMGGKPQRRKNPQE |
| | GLYNELQKDKMAEAYSEIGMKGERRRGK |
| | GHDGLYQGLSTATKDTYDALHMQALPPR |
| | (SEQ ID NO: 22) |

A typical number of cells, e.g., immune cells or hematopoietic cells, administered to a mammal (e.g., a human) can be, for example, in the range of one million to 100 billion cells; however, amounts below or above this exemplary range are also within the scope of the present disclosure.

In some embodiments, the agent that targets CD33 is an antibody-drug conjugate (ADC). The ADC may be a molecule comprising an antibody or antigen-binding fragment thereof conjugated to a toxin or drug molecule. Binding of the antibody or fragment thereof to the corresponding antigen allows for delivery of the toxin or drug molecule to a cell that presents the antigen on the its cell surface (e.g., target cell), thereby resulting in death of the target cell.

In some embodiments, the antigen-bind fragment of the antibody-drug conjugate has the same heavy chain CDRs as the heavy chain variable region provided by SEQ ID NO: 15 and the same light chain CDRs as the light chain variable region provided by SEQ ID NO: 16. In some embodiments, the antigen-bind fragment of the antibody-drug conjugate has the heavy chain variable region provided by SEQ ID NO: 15 and the same light chain variable region provided by SEQ ID NO: 16.

Toxins or drugs compatible for use in antibody-drug conjugates are known in the art and will be evident to one of ordinary skill in the art. See, e.g., Peters et al. Biosci. Rep. (2015) 35(4): e00225; Beck et al. Nature Reviews Drug Discovery (2017) 16:315-337; Marin-Acevedo et al. J. Hematol. Oncol. (2018) 11: 8; Elgundi et al. Advanced Drug Delivery Reviews (2017) 122: 2-19.

In some embodiments, the antibody-drug conjugate may further comprise a linker (e.g., a peptide linker, such as a cleavable linker) attaching the antibody and drug molecule. Examples of antibody-drug conjugates include, without limitation, brentuximab vedotin, glembatumumab vedotin/CDX-011, depatuxizumab mafodotin/ABT-414, PSMA ADC, polatuzumab vedotin/RG7596/DCDS4501A, denintuzumab mafodotin/SGN-CD19A, AGS-16C3F, CDX-014, RG7841/DLYE5953A, RG7882/DMUC406A, RG7986/DCDS0780A, SGN-LIV1A, enfortumab vedotin/ASG-22ME, AG-15ME, AGS67E, telisotuzumab vedotin/ABBV-399, ABBV-221, ABBV-085, GSK-2857916, tisotumab vedotin/HuMax-TF-ADC, HuMax-Axl-ADC, pinatuzumab veodtin/RG7593/DCDT2980S, lifastuzumab vedotin/RG7599/DNIB0600A, indusatumab vedotin/MLN-0264/TAK-264, vandortuzumab vedotin/RG7450/DSTP3086S, sofituzumab vedotin/RG7458/DMUC5754A, RG7600/DMOT4039A, RG7336/DEDN6526A, ME1547, PF-06263507/ADC 5T4, trastuzumab emtansine/T-DM1, mirvetuximab soravtansine/IMGN853, coltuximab ravtansine/SAR3419, naratuximab emtansine/IMGN529, indatuximab ravtansine/BT-062, anetumab ravtansine/BAY 94-9343, SAR408701, SAR428926, AMG 224, PCA062, HKT288, LY3076226, SAR566658, lorvotuzumab mertansine/IMGN901, cantuzumab mertansine/SB-408075, cantuzumab ravtansine/IMGN242, laprituximab emtansine/IMGN289, IMGN388, bivatuzumab mertansine, AVE9633, BIIB015, MLN2704, AMG 172, AMG 595, LOP 628, vadastuximab talirine/SGN-CD33A, SGN-CD70A, SGN-CD19B, SGN-CD123A, SGN-CD352A, rovalpituzumab tesirine/SC16LD6.5, SC-002, SC-003, ADCT-301/HuMax-TAC-PBD, ADCT-402, MEDI3726/ADC-401, IMGN779, IMGN632, gemtuzumab ozogamicin, inotuzumab ozogamicin/CMC-544, PF-06647263, CMD-193, CMB-401, trastuzumab duocarmazine/SYD985, BMS-936561/MDX-1203, sacituzumab govitecan/IMMU-132, labetuzumab govitecan/IMMU-130, DS-8201a, U3-1402, milatuzumab doxorubicin/IMMU-110/hLL1-DOX, BMS-986148, RC48-ADC/hertuzumab-vc-MMAE, PF-06647020, PF-06650808, PF-06664178/RN927C, lupartumab amadotin/BAY1129980, aprutumab ixadotin/BAY1187982, ARX788, AGS62P1, XMT-1522, AbGn-107, MEDI4276, DSTA4637S/RG7861. In one example, the antibody-drug conjugate is gemtuzumab ozogamicin.

In some embodiments, binding of the antibody-drug conjugate to the epitope of the cell-surface lineage-specific protein induces internalization of the antibody-drug conjugate, and the drug (or toxin) may be released intracellularly. In some embodiments, binding of the antibody-drug conjugate to the epitope of a cell-surface lineage-specific protein induces internalization of the toxin or drug, which allows the toxin or drug to kill the cells expressing the lineage-specific protein (target cells). In some embodiments, binding of the antibody-drug conjugate to the epitope of a cell-surface lineage-specific protein induces internalization of the toxin or drug, which may regulate the activity of the cell expressing the lineage-specific protein (target cells). The type of toxin or drug used in the antibody-drug conjugates described herein is not limited to any specific type.

### EXAMPLES

### Example 1: Genetic Editing of CD33 in Human Cells

Design of sgRNA Constructs

The sgRNAs indicated in Table 4 were designed by manual inspection for the SpCas9 PAM (5′-NGG-3′) with close proximity to the target region and prioritized according to predicted specificity by minimizing potential off-target sites in the human genome with an online search algorithm (e.g., the Benchling algorithm, Doench et al 2016, Hsu et al 2013). All designed synthetic sgRNAs were produced with chemically modified nucleotides at the three terminal positions at both the 5′ and 3′ ends. Modified nucleotides contained 2′-O-methyl-3′-phosphorothioate (abbreviated as “ms”) and the ms-sgRNAs were HPLC-purified. Cas9 protein was purchased from Synthego.

| | | | |
|---|---|---|---|
| gRNA A | CCCCAGGACTACTCACTCCT (SEQ ID NO: 1) | CGG | Exon 3 |
| gRNA B | ACCGAGGAGTGAGTAGTCCT (SEQ ID NO: 2) | GGG | Exon 3 |

### Editing in Primary Human CD34+ HSCs

Frozen CD34+ HSCs derived from mobilized peripheral blood were purchased either from Hemacare or Fred Hutchinson Cancer Center and thawed according to manufacturer's instructions. To edit HSCs, ˜1×10⁶ HSCs were thawed and cultured in StemSpan SFEM medium supplemented with StemSpan CC110 cocktail (StemCell Technologies) for 24-48 h before electroporation with RNP. To electroporate HSCs, 1.5×10⁵ cells were pelleted and resuspended in 20 μL Lonza P3 solution, and mixed with 10 μL Cas9 RNP. CD34+ HSCs were electroporated using the Lonza Nucleofector 2 (program DU-100) and the Human P3 Cell Nucleofection Kit (VPA-1002, Lonza).

### Genomic DNA Analysis

For all genomic analysis, DNA was harvested from cells, amplified with primers flanking the target region, purified and the allele modification frequencies were analyzed using TIDE (Tracking of Indels by Decomposition). Analyses were performed using a reference sequence from a mock-transfected sample. Parameters were set to the default maximum indel size of 10 nucleotides and the decomposition window to cover the largest possible window with high quality traces. All TIDE analyses below the detection sensitivity of 3.5% were set to 0%.

### Flow Cytometry Analysis

Live HL60 or CD34+ HSCs were stained for CD33 using an anti-CD33 antibody (P67.7) and analyzed by flow cytometry on the Attune NxT flow cytometer (Life Technologies).

### Results

Human CD34+ cells were electroporated with Cas9 protein and indicated CD33-targeting gRNA as described above.

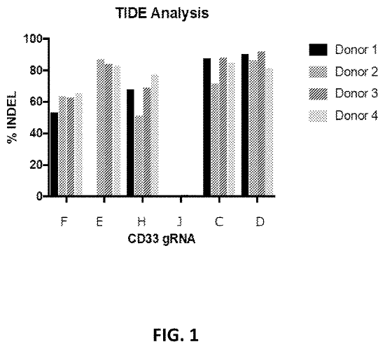

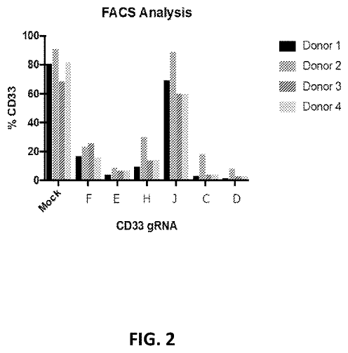

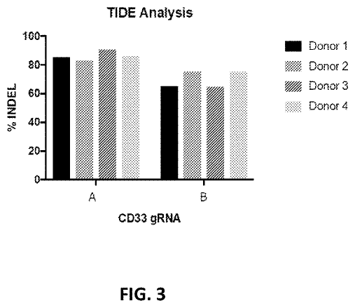

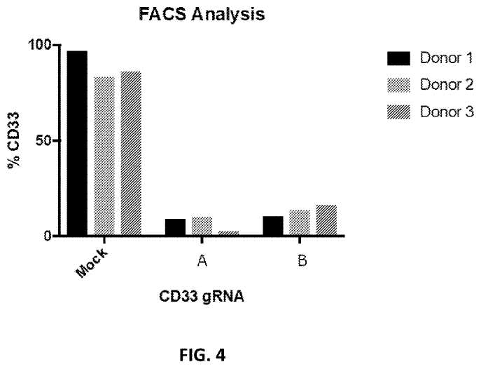

The percentage editing was determined by % INDEL as assessed by TIDE (FIG. 1 and FIG. 3) or surface CD33 protein expression by flow cytometry (FIG. 2 and FIG. 4). Editing efficiency was determined from the flow cytometric analysis.

As shown in FIG. 1, gRNA F, gRNA 55E, gRNA H, gRNA C, and gRNA D gave a high proportion of indels, in the range of approximately 50-100% of cells. In comparison, gRNA J gave a much lower proportion of indels. gRNA E showed a high indel frequency in Donors 2-4 but not in Donor 1. The other gRNAs of FIG. 1 showed more similar results from donor to donor.

As shown in FIG. 2, gRNAs gRNA F, gRNA E, gRNA H, gRNA C, and gRNA D showed a marked reduction in CD33 expression as detected by FACS. gRNA J did not show a similar reduction in CD33 expression, consistent with its lower activity observed in FIG. 1.

As shown in FIG. 3, gRNA A and gRNA B gave a high proportion of indels, in the range of approximately 60-90% of cells.

As shown in FIG. 4, gRNA A and gRNA B showed a marked reduction in CD33 expression as detected by FACS.

The gene editing efficiency of a subset of these, and other, gRNAs was tested in the HL60 AML (promyeloblast leukemia) cell line. The HL-60 cells were genetically edited via CRISPR/Cas9 using the indicated gRNAs. The percentage of CD33-positive cells were assessed by flow cytometry 6 days post electroporation to assess effectiveness in knocking out CD33. Genomic DNA was PCR amplified and analyzed by TIDE as described above to determine the percentage editing as assessed by INDEL (insertion/deletion).

TABLE 5
Gene editing efficiency of CD33 gRNAs.

| gRNA | % CD33+ (FACS) | % INDEL (TIDE) |
|---|---|---|
| Mock | 99.6% | n/a |
| gRNA F | 10.2% | 87.3% |
| gRNA E | 9.73% | 89.9% |
| gRNA G | 45.2% | 47.1% |
| gRNA H | 13.8% | 88.6% |
| gRNA I | 14.8% | 58.1% |
| gRNA J | 99.7% | — |
| gRNA C | 7.9% | 77.7% |
| gRNA K | 8.6% | 77.3% |
| gRNA L | 17.1% | 71.7% |
| gRNA D | 5.4% | 93% |
| gRNA M | 8.5% | 32.9% |
| gRNA N | 65.1% | 52.5% |

### Example 2: Efficient Multiplex Genomic Editing

Efficient double genomic editing of CD19 and CD33 genes in HSC cells were performed in either NALM-6 cells or in HSCs following conventional methods or those described herein. Table 8 below provides the gRNAs targeting exon 2 of CD19 and exon 3 of CD33.

TABLE 6
Guide RNA Targeting Domain Sequences for Double Editing of CD19 and
CD33. A corresponding gRNA can comprise an equivalent RNA sequence.

| Gene | Sequence | PAM | Location |
|---|---|---|---|
| CD19 | CACAGCGTTATCTCCCTCTG | GGT | Exon 2 |
| | (SEQ ID NO: 62) | | |
| CD33 (gRNA A) | CCCCAGGACTACTCACTCCT | CGG | Exon 3 |
| | (SEQ ID NO: 1) | | |

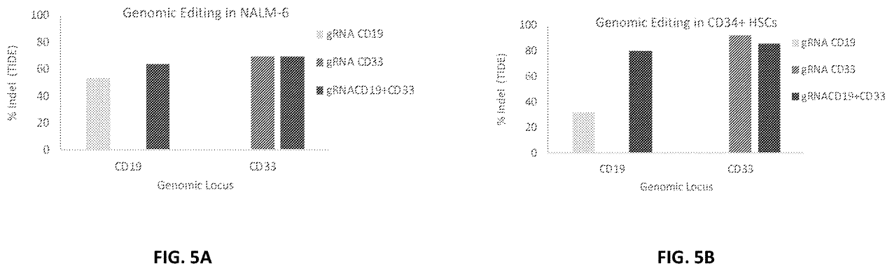

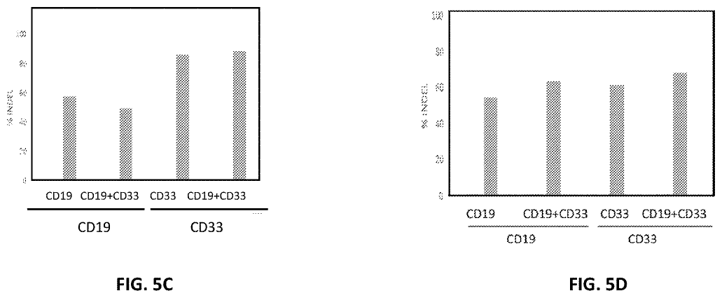

Genomic DNA was isolated from bulk-edited cells and TIDE assays were performed to examine genomic editing in NALM-6, HL-60 cells and HSCs. Results are depicted in FIGS. 5A-5D. The results obtained from this study show that ˜70% of the HSCs include mutations in both loci of the CD19 gene and −80% of the HSCs include mutations in both loci of the CD33 gene, indicating that at least 50% of the double-edited cells have both edited CD19 gene and edited CD33 gene on at least one chromosome. Similar levels of edited cells were observed in HL-60 cells and Nalm-6 cells.

### Example 3: Effect of Editing Multiple Loci on Viability

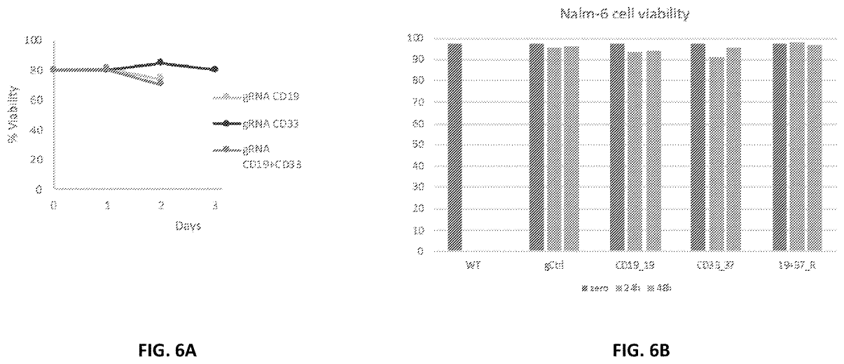

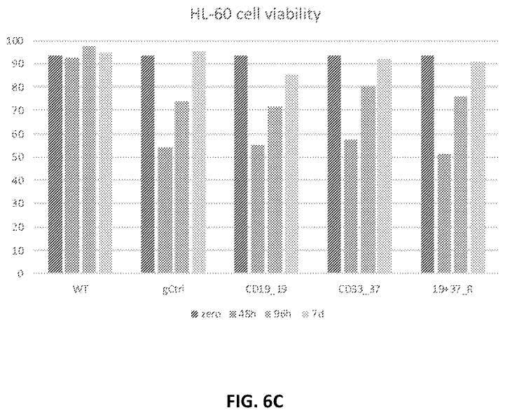

The effect of genomic editing at multiple loci on cell viability was assessed NALM-6, HL-60 cells and HSCs. Two days before nucleofection, HSPCs were thawed or Nalm-6 or HL-60 cells were passaged. At 24 and 48 hours (day of nucleofection), cells were counted and cell viability was assessed. Nucleofection performed with complexes comprising the gRNAs of Table 6 and was performed according to Materials and Methods described herein. Cells were counted and cell viability was assessed at the indicated timepoints. Results are depicted in FIGS. 6A-6C and indicate that dual Cas9/gRNAs delivery does not impair viability in cell lines. No additional toxicity over single guide RNA was observed in HSCs.

### Example 4: Generation and Evaluation of Cells Edited for Two Cell Surface Antigens

### Results

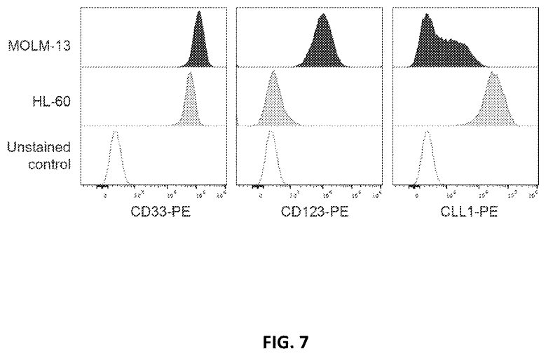

Cell surface levels of CD33, CD123 and CLL1 (CLEC12A) were measured in unedited MOLM-13 cells and THP-1 cells (both human AML cell lines) by flow cytometry. MOLM-13 cells had high levels of CD33 and CD123, and moderate-to-low levels of CLL1. HL-60 cells had high levels of CD33 and CLL1, and low levels of CD123 (FIG. 7).

CD33 and CD123 were mutated in MOLM-13 cells using gRNAs and Cas9 as described herein, CD33 and CD123-modified cells were purified by flow cytometric sorting, and the cell surface levels of CD33 and CD123 were measured. CD33 and CD123 levels were high in wild-type MOLM-13 cells; editing of CD33 only resulted in low CD33 levels;

editing of CD123 only resulted in low CD123 levels, and editing of both CD33 and CD123 resulted in low levels of both CD33 and CD123 (FIG. 8). The edited cells were then tested for resistance to CART effector cells using an in vitro cytotoxicity assay as described herein. All four cell types (wild-type, CD33^{-/-}, CD123^{-/-}, and CD33^{-/-} CD123^{-/-}) experienced low levels of specific killing in mock CAR control conditions (FIG. 9, leftmost set of bars). CD33 CAR cells effectively killed wild-type and CD123^{-/-} cells, while CD33^{-/-} and CD33^{-/-} CD123^{-/-} cells showed a statistically significant resistance to CD33 CAR (FIG. 9, second set of bars). CD123 CAR cells effectively killed wild-type and CD33^{-/-} cells, while CD123^{-/-} and CD33^{-/-}CD123^{-/-} cells showed a statistically significant resistance to CD123 CAR (FIG. 9, third set of bars). A pool of CD33 CAR and CD123 CAR cells effectively killed wild-type cells, CD33^{-/-} cells, and CD123^{-/-} cells, while CD33^{-/-}CD123^{-/-} cells showed a statistically significant resistance to the pool of CAR cells (FIG. 9, rightmost set of bars). This experiment demonstrates that knockout of two antigens (CD33 and CD123) protected the cells against CAR cells targeting both antigens. Furthermore, the population of edited cells contained a high enough proportion of cells that were edited at both alleles of both antigens, and had sufficiently low cell surface levels of cell surface antigens, that a statistically significant resistance to both types of CAR cells was achieved.

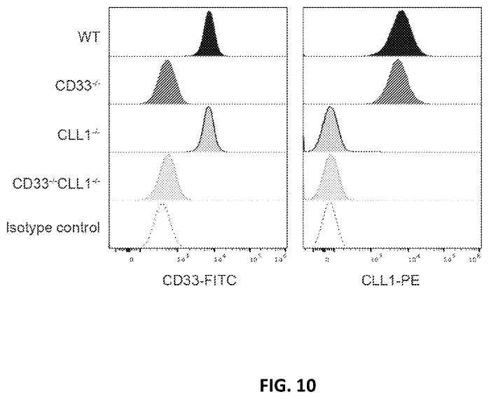

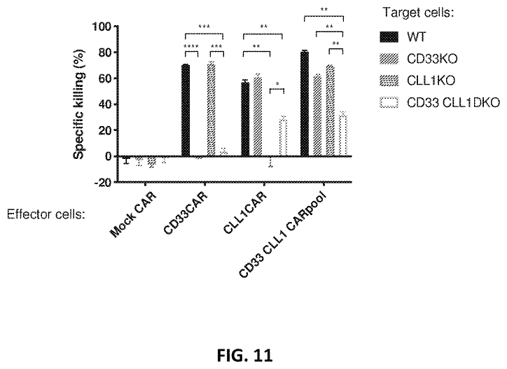

CD33 and CLL1 were mutated in HL-60 using gRNAs and Cas9 as described herein, CD33 and CLL1-modified cells were purified by flow cytometric sorting, and the cell surface levels of CD33 and CLL1 were measured. CD33 and CLL1 levels were high in wild-type HL-60 cells; editing of CD33 only resulted in low CD33 levels; editing of CLL1 only resulted in low CLL1 levels, and editing of both CD33 and CLL1 resulted in low levels of both CD33 and CLL1 (FIG. 10). The edited cells were then tested for resistance to CART effector cells using an in vitro cytotoxicity assay as described herein. All four cell types (wild-type, CD33^{-/-}, CLL1^{-/-}, and CD33^{-/-} CLL1^{-/-}) experienced low levels of specific killing in mock CAR control conditions (FIG. 11, leftmost set of bars). CD33 CAR cells effectively killed wild-type and CLL1^{-/-} cells, while CD33^{-/-} and CD33^{-/-} CLL1^{-/-} cells showed a statistically significant resistance to CD33 CAR (FIG. 11, second set of bars). CLL1 CAR cells effectively killed wild-type and CD33^{-/-} cells, while CLL1^{-/-} and CD33^{-/-} CLL1^{-/-} cells showed a statistically significant resistance to CLL1 CAR (FIG. 11, third set of bars). A pool of CD33 CAR and CLL1 CAR cells effectively killed wild-type cells, CD33^{-/-} cells, and CLL1^{-/-} cells, while CD33^{-/-} CLL1^{-/-} cells showed a statistically significant resistance to the pool of CAR cells (FIG. 11, rightmost set of bars). This experiment demonstrates that knockout of two antigens (CD33 and CLL1) protected the cells against CAR cells targeting both antigens. Furthermore, the population of edited cells contained a high enough proportion of cells that were edited at both alleles of both antigens, and had sufficiently low cell surface levels of cell surface antigens, that a statistically significant resistance to both types of CAR cells was achieved.

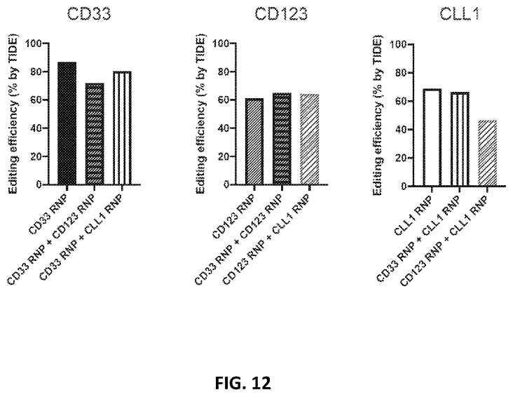

The efficiency of gene editing in human CD34+ cells was quantified using TIDE analysis as described herein. At the endogenous CD33 locus, editing efficiency of between about 70-90% was observed when CD33 was targeted alone or in combination with CD123 or CLL1 (FIG. 12, left graph). At the endogenous CD123 locus, editing efficiency of about 60% was observed when CD123 was targeted alone or in combination with CD33 or CLL1 (FIG. 12, center graph). At the endogenous CLL1 locus, editing efficiency of between about 40-70% was observed when CLL1 was targeted alone or in combination with CD33 or CD123 (FIG. 12, right graph). This experiment illustrates that human CD34+ cells can be edited at a high frequency at two cell surface antigen loci.

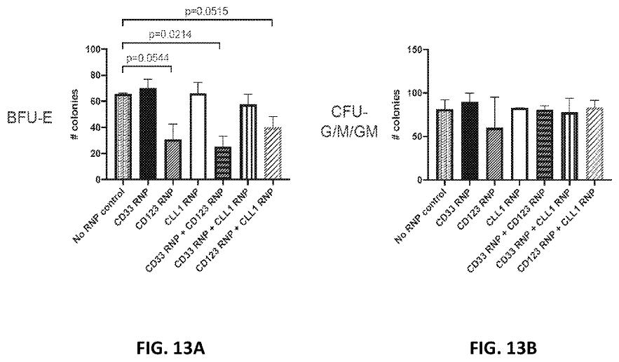

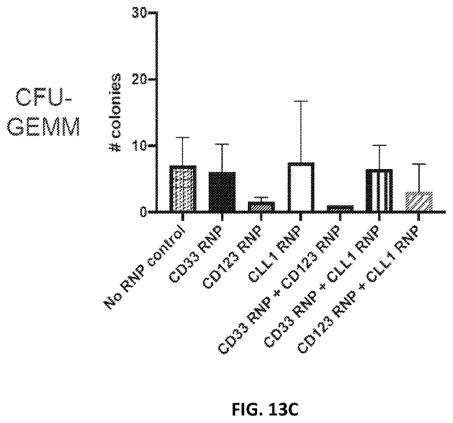

The differentiation potential of gene-edited human CD34+ cells as measured by colony formation assay as described herein. Cells edited for CD33, CD123, or CLL1, individually or in all pairwise combinations, produced BFU-E colonies (Burst forming unit-erythroid), showing that the cells retain significant differentiation potential in this assay (FIG. 13A). The edited cells also produced CFU-G/M/GM colonies, showing that the cells retain differentiation potential in this assay that is statistically indistinguishable from the non-edited control (FIG. 13B). The edited cells also produced detectable CFU-GEMM colonies (FIG. 13C). Colony forming unit (CFU)-G/M/GM colonies refer to CFU-G (granulocyte), CFU-M (macrophage), and CFU-GM (granulocyte/macrophage) colonies. CFU-GEMM (granulocyte/erythroid/macrophage/megakaryocyte) colonies arise from a less differentiated cell that is a precursor to the cells that give rise to CFU-GM colonies. Taken together, the differentiation assays indicate that human CD34+ cells edited at two loci retain the capacity to differentiate into variety of cell types.

### Materials and Methods

### AML Cell Lines

Human AML cell line HL-60 was obtained from the American Type Culture Collection (ATCC). HL-60 cells were cultured in Iscove's Modified Dulbecco's Medium (IMDM, Gibco) supplemented with 20% heat-inactivated HyClone Fetal Bovine Serum (GE Healthcare). Human AML cell line MOLM-13 was obtained from AddexBio Technologies. MOLM-13 cells were cultured in RPMI-1640 media (ATCC) supplemented with 10% heat-inactivated HyClone Fetal Bovine Serum (GE Healthcare).

### Guide RNA Design

All sgRNAs were designed by manual inspection for the SpCas9 PAM (5′-NGG-3′) with close proximity to the target region and prioritized according to predicted specificity by minimizing potential off-target sites in the human genome with an online search algorithm (e.g., the Benchling algorithm, Doench et al 2016, Hsu et al 2013). All designed synthetic sgRNAs were purchased from Synthego with chemically modified nucleotides at the three terminal positions at both the 5′ and 3′ ends. Modified nucleotides contained 2′-O-methyl-3′-phosphorothioate (abbreviated as “ms”) and the ms-sgRNAs were HPLC-purified. Cas9 protein was purchased from Aldervon. Typically, the gRNAs described in the Examples herein are sgRNAs comprising a 20 nucleotide (nt) targeting sequence, 12 nt of the crRNA repeat sequence, 4 nt of tetraloop sequence, and 64 nt of tracrRNA sequence.

TABLE 14
sequences of targeting domains of gRNAs targeting CD33, CD123, or
CLL-1. A corresponding gRNA can comprise an equivalent RNA sequence.

| Target gene | Sequence | PAM | Target location |
|---|---|---|---|
| CD33 (gRNA A) | CCCCAGGACTACTCACTCCT | CGG | CD33 exon 3 |
| | (SEQ ID NO: 1) | | |
| CD123 | TTTCTTGAGCTGCAGCTGGG | CGG | CD123 exon 5 |
| | (SEQ ID NO: 24) | | |
| | AGTTCCCACATCCTGGTGCG | GGG | CD123 exon 6 |
| | (SEQ ID NO: 25) | | |
| CLL1 | GGTGGCTATTGTTTGCAGTG | TGG | CLL1 exon 4 |
| | (SEQ ID NO: 23) | | |

### AML Cell Line Electroporation

Cas9 protein and ms-sgRNA (at a 1:1 weight ratio) were mixed and incubated at room temperature for 10 minutes prior to electroporation. MOLM-13 and HL-60 cells were electroporated with the Cas9 ribonucleoprotein complex (RNP) using the MaxCyte ATx Electroporator System with program THP-1 and Opt-3, respectively. Cells were incubated at 37° C. for 5-7 days until flow cytometric sorting.

### Human CD34+ Cell Culture and Electroporation

Cryopreserved human CD34+ cells were purchased from Hemacare and thawed according to manufacturer's instructions. Human CD34+ cells were cultured for 2 days in GMP SCGM media (CellGenix) supplemented with human cytokines (Flt3, SCF, and TPO, all purchased from Peprotech). CD34+ cells were electroporated with the Cas9 RNP (Cas9 protein and ms-sgRNA at a 1:1 weight ratio) using Lonza 4D-Nucleofector and P3 Primary Cell Kit (Program CA-137). For electroporation with dual ms-sgRNAs, equal amount of each ms-sgRNA was added. Cells were cultured at 37° C. until analysis.

### Genomic DNA Analysis

Genomic DNA was extracted from cells 2 days post electroporation using prepGEM DNA extraction kit (ZyGEM). Genomic region of interest was amplified by PCR.

PCR amplicons were analyzed by Sanger sequencing (Genewiz) and allele modification frequency was calculated using TIDE (Tracking of Indels by Decomposition) software available on the World Wide Web at tide.deskgen.com.

### In Vitro Colony Forming Unit (CFU) Assay

Two days after electroporation, 500 CD34+ cells were plated in 1.1 mL of methylcellulose (MethoCult H4034 Optimum, Stem Cell Technologies) on 6 well plates in duplicates and cultured for two weeks. Colonies were then counted and scored using StemVision (Stem Cell Technologies).

### Flow Cytometric Analysis and Sorting

Flurochrome-conjugated antibodies against human CD33 (P67.6), CD123 (9F5), and CLL1 (REA431) were purchased from Biolegend, BD Biosciences and Miltenyi Biotec, respectively. All antibodies were tested with their respective isotype controls. Cell surface staining was performed by incubating cells with specific antibodies for 30 min on ice in the presence of human TruStain FcX. For all stains, dead cells were excluded from analysis by DAPI (Biolegend) stain. All samples were acquired and analyzed with Attune NxT flow cytometer (ThermoFisher Scientific) and FlowJo software (TreeStar).

For flow cytometric sorting, cells were stained with flurochrome-conjugated antibodies followed by sorting with Moflow Astrios Cell Sorter (Beckman Coulter).

### CAR Constructs and Lentiviral Production

Second-generation CARs were constructed to target CD33, CD123, and CLL-1, with the exception of the anti-CD33 CAR-T used in CD33/CLL-1 multiplex cytotoxicity experiment. Each CAR consisted of an extracellular scFv antigen-binding domain, using CD8α signal peptide, CD8α hinge and transmembrane regions, the 4-1BB costimulatory domain, and the CD3ξ signaling domain. The anti-CD33 scFv sequence was obtained from clone P67.6 (Mylotarg); the anti-CD123 scFv sequence from clone 32716; and the CLL-1 scFv sequence from clone 1075.7. The anti-CD33 and anti-CD123 CAR constructs uses a heavy-to-light orientation of the scFv, and the anti-CLL1 CAR construct uses a light-to-heavy orientation. The heavy and light chains were connected by (GGGS)3 linker (SEQ ID NO: 63). CAR cDNA sequences for each target were sub-cloned into the multiple cloning site of the pCDH-EF1α-MCS-T2A-GFP expression vector, and lentivirus was generated following the manufacturer's protocol (System Biosciences). Lentivirus can be generated by transient transfection of 293TN cells (System Biosciences) using Lipofectamine 3000 (ThermoFisher). The CAR construct was generated by cloning the light and heavy chain of anti-CD33 scFv (clone My96), to the CD8α hinge domain, the ICOS transmembrane domain, the ICOS signaling domain, the 4-1BB signaling domain and the CD3ξ signaling domain into the lentiviral plasmid pHIV-Zsgreen.

### CAR Transduction and Expansion

Human primary T cells were isolated from Leuko Pak (Stem Cell Technologies) by magnetic bead separation using anti-CD4 and anti-CD8 microbeads according to the manufacturer's protocol (Stem Cell Technologies). Purified CD4+ and CD8+ T cells were mixed 1:1, and activated using anti-CD3/CD28 coupled Dynabeads (Thermo Fisher) at a 1:1 bead to cell ratio. T cell culture media used was CTS Optimizer T cell expansion media supplemented with immune cell serum replacement, L-Glutamine and GlutaMAX (all purchased from Thermo Fisher) and 100 IU/mL of IL-2 (Peprotech). T cell transduction was performed 24 hours post activation by spinoculation in the presence of polybrene (Sigma). CAR-T cells were cultured for 9 days prior to cryopreservation. Prior to all experiments, T cells were thawed and rested at 37° C. for 4-6 hours.

### Flow Cytometry Based CAR-T Cytotoxicity Assay

The cytotoxicity of target cells was measured by comparing survival of target cells relative to the survival of negative control cells. For CD33/CD123 multiplex cytotoxicity assays, wildtype and CRISPR/Cas9 edited MOLM-13 cells were used as target cells, while wildtype and CRISPR/Cas9 edited HL60 cells were used as target cells for CD33/CLL-1 multiplex cytotoxicity assays. Wildtype Raji cell lines (ATCC) were used as negative control for both experiments. Target cells and negative control cells were stained with CellTrace Violet (CTV) and CFSE (Thermo Fisher), respectively, according to the manufacturer's instructions. After staining, target cells and negative control cells were mixed at 1:1.

Anti-CD33, CD123, or CLL1 CAR-T cells were used as effector T cells. Non-transduced T cells (mock CAR-T) were used as control. For the CARpool groups, appropriate CAR-T cells were mixed at 1:1. The effector T cells were co-cultured with the target cell/negative control cell mixture at a 1:1 effector to target ratio in duplicate. A group of target cell/negative control cell mixture alone without effector T cells was included as control. Cells were incubated at 37° C. for 24 hours before flow cytometric analysis. Propidium iodide (ThermoFisher) was used as a viability dye. For the calculation of specific cell lysis, the fraction of live target cell to live negative control cell (termed target fraction) was used. Specific cell lysis was calculated as ((target fraction without effector cells—target fraction with effector cells)/(target fraction without effectors))×100%.

### Example 5: Effect of Gemtuzumab Ozogamycin on Engineered HSCs

Frozen CD34+ HSPCs derived from mobilized peripheral blood were thawed and cultured for 72 h before electroporation with ribonucleoprotein comprising Cas9 and an sgRNA. Samples were electroporated with the following conditions:

i.) Mock (Cas9 only),

ii. KO sgRNA (CD33 gRNA A)

Cells were allowed to recover for 72 hours and genomic DNA was collected and analyzed.

The percentage of CD33-positive cells were assessed by flow cytometry, confirming that editing with gRNA A was effective in knocking out CD33 (data not shown). The editing events in the HSCs were found to result in a variety of indel sequences (data not shown).

(i) Sensitivity of Cells Having CD33exon2 Deletion to Gemtuzumab Ozogramicin (GO)

To determine in vitro toxicity, cells were incubated with GO in their culture media and the number of viable cells was quantified over time. As shown in the table below, CD33 knockout cells generated with CD33 gRNA A were more resistant to GO treatment than cells expressing full length CD33 (mock). 50% editing observed in CD33KO cells is considered sufficient protection in dividing cells.

| | After 3 days | | After 7 days | | After 15 days | |
|---|---|---|---|---|---|---|
| | Cell | | Cell | | Cell | |
| | number | | number | | number | |
| Geno- | (million/ | % | (million/ | % | (million/ | % |
| type | ml) | viable | ml) | viable | ml) | viable |
| Mock | 0.8 | 90 | 0.65 | 97 | 0.43 | 97 |
| CD33 | 1.4 | 90 | 1.54 | 96 | 1.0 | 97 |
| gRNA | | | | | | |
| A | | | | | | |

(ii) Enrichment of CD33-Modified Cells

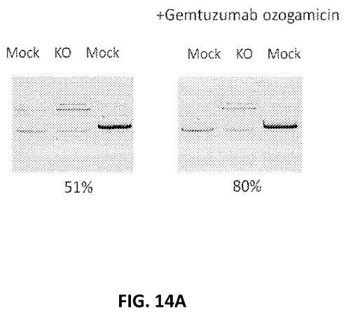

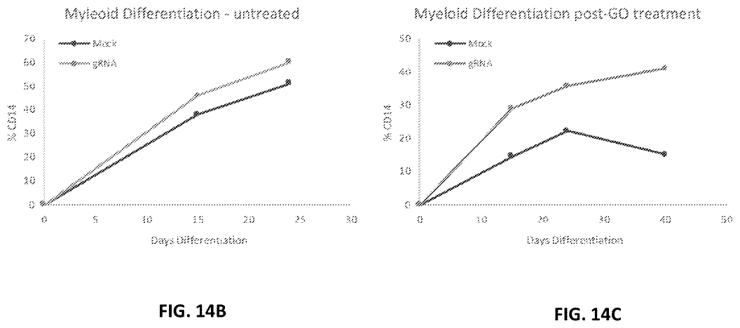

To assay if CD33 modified cells were enriched following GO-treatment, CD34+ HSPCs were edited with 50% of standard Cas9/gRNA ratios. The bulk population of cells were analyzed prior to and after GO treatment. As shown in FIG. 14A, prior to GO treatment, 51% of gRNA A modified cells (KO) as assayed by TIDE. Following GO-treatment, CD33 modified cells were enriched so that the percentage of KO cells increased to 80%. This data indicated that there was an enrichment of CD33 modified cells following GO-treatment.

(iii) In Vitro Differentiation of CD34+ HSPCs

Cell populations were assessed for myeloid differentiation prior to and after GO treatment at various days post differentiation. As shown in FIGS. 14B and 14C, CD33 knockout cells generated with CD33 gRNA A showed increased expression of the differentiation marker, CD14, whereas cells expressing full length CD33 (mock) did not differentiate.

### Example 6: Evaluation of the Persistence of CD33KO CD34+ Cells In Vivo

Editing in mobilized peripheral blood CD34+ HSCs (mPB CD34+ HSPCs) gRNAs (Synthego) were designed as described in Example 1. mPB CD34+ HSPCs were purchased from Fred Hutchinson Cancer Center and thawed according to manufacturer's instructions. These cells were then edited via CRISPR/Cas9 as described in Example 1 using the CD33-targeting guide RNAs: gRNA A (SEQ ID NO: 1), gRNA B (SEQ ID NO: 2), gRNA O(CCTCACTAGACTTGACCCAC) (SEQ ID NO: 64), as well as a non-CD33 targeting control gRNA (gCtrl) that was designed not to target any region in the human or mouse genomes.

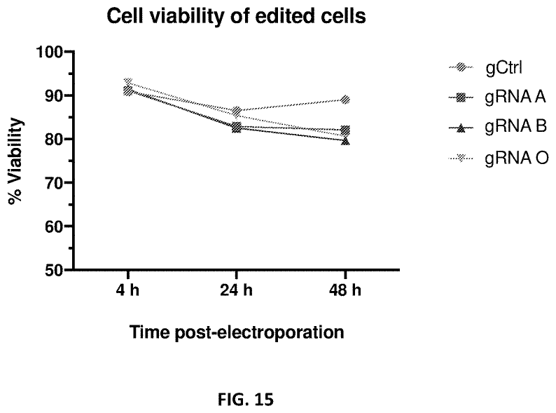

At 4, 24, and 48 hours post-ex vivo editing, the percentages of viable, edited CD33KO cells and control cells were quantified using flow cytometry and the 7AAD viability dye (FIG. 15). As shown in FIG. 15, high levels of CD33KO cells edited using all three gRNAs (A, B, or O) were viable (approximately 80-95% viable cells observed), and remained viable over time following electroporation and gene editing. This was similar to what was observed in the control cells edited with the non-CD33 targeting control gRNA, gCtrl.

Additionally, at 48 hours post-ex vivo editing, the genomic DNA was harvested from cells, PCR amplified with primers flanking the target region, purified, and analyzed by TIDE, in order to determine the percentage editing as assessed by INDEL (insertion/deletion). As shown in Table 10, gRNA A and gRNA B gave a high proportion of indels, specifically 93.1% and 91.3%, respectively. This was comparable to the proportion of indels from the control, CD33-targeting gRNA, gRNA O.

TABLE 10
Gene editing efficiency of CD33 gRNAs.

| gRNA | % INDEL (TIDE) |
|---|---|
| gRNA A | 93.1% |
| gRNA B | 81.3% |
| gRNA O | 92.7% |

Following TIDE analysis, the percentage of long term-HSCs (LT-HSCs) following editing with the indicated CD33 targeting gRNAs was quantified by Flow cytometry, gating for cells that were CD38−CD34+CD45RA−CD90+CD49f+. The percentages of LT-HSCs following editing with the specified gRNAs are presented in Table 11. This assay was done at the time of cryopreservation of the edited cells, prior to injection into mice for investigation of persistence of CD33KO cells in vivo. The edited cells were cryopreserved in CS10 media (Stem Cell Technology) at 5×10⁶ cells/mL, in a 1 mL volume of media per vial.

TABLE 11
Quantification of LT-HSC population following
ex vivo editing of CD34+ HSCs

| Group | Cells | % LT-HSC | % of total population |
|---|---|---|---|
| 1 | gCtrl | 39.4 | 2.5 |
| 2 | gRNA O | 36.2 | 2.03 |
| 3 | gRNA A | 39 | 2.3 |
| 4 | gRNA B | 36.6 | 2.1 |

### Investigating Engraftment Efficiency and Persistence of CD33KO mPB CD34+ HSPCs In Vivo

Female NSG mice (JAX) that were 6 to 8 weeks of age, were allowed to acclimate for 2-7 days. Following acclimation, mice were irradiated using 175 cGy whole body irradiation by X-ray irradiator. This was regarded as day 0 of the investigation. At 4-10 hours, following irradiation, the mice were engrafted with the CD33KO edited with gRNA A, gRNA B, or gRNA O or control cells edited with gCtrl (n=15). The cryopreserved cells were thawed and counted using a BioRad TC-20 automated cell counter. The number of viable cells was quantified in the thawed vials, which was used to prepare the total number of cells for engraftment in the mice (Table 12). Mice were given a single intravenous injection of 1×10⁶ edited cells in a 100 μL volume. Body weight and clinical observations were recorded once weekly for each mouse in the four groups.

TABLE 12
Viability of thawed edited CD33KO cells and control cells

| | | | Expected # of total | Calculated # of |
|---|---|---|---|---|
| | | Vials | cells (5 × 10⁶ per | total viable cells |
| Group | Cells | Thawed | vial) | in thawed vials |
| 1 | gCtrl | 5 | 25 × 10⁶ | 22.4 × 10⁶ |
| | | | | (87% viability) |
| 2 | gRNA O | 5 | 25 × 10⁶ | 19.7 × 10⁶ |
| | | | | (80.5% viability) |
| 3 | gRNA A | 5 | 25 × 10⁶ | 24.4 × 10⁶ |
| | | | | (82% viability) |
| 4 | gRNA B | 5 | 25 × 10⁶ | 24.7 × 10⁶ |
| | | | | (80.5% viability) |

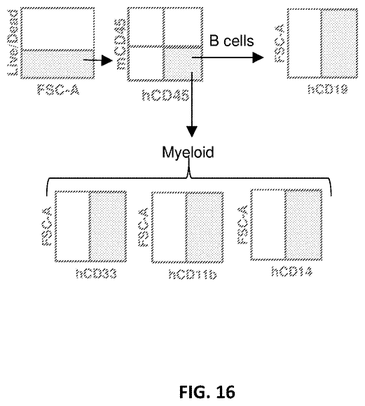

At weeks 8 and 12 following engraftment, 50 μL of blood was collected from each mouse by retroorbital bleed for analysis by flow cytometry. At week 16, following engraftment, mice were sacrificed and blood, spleens, and bone marrow were collected for analysis by flow cytometry. Bone marrow was isolated from the femur and the tibia. Bone marrow from the femur was also used for on-target editing analysis. The markers measured by flow cytometry and the antibodies (Biolegend or BD Bioscience) used are denoted in Table 13. Flow cytometry was performed using the FACSCanto™ 10 color and BDFACSDiva™ software. As depicted in the schematic of the flow cytometry experimental design and gating protocol in FIG. 16, cells were first sorted by viability using the 7AAD viability dye (live/dead analysis). Live cells were then gated by expression of human CD45 (hCD45) but not mouse CD45 (mCD45). These cells that were hCD45+ were then further gated for the expression of human CD19 (hCD19) (lymphoid cells, specifically B cells). Cells expressing human CD45 (hCD45) were also gated and analyzed for the presence of for various cellular markers of the myeloid lineage, including, at least hCD33, hCD11b, and hCD14.

TABLE 13
Markers used for quantification of cells
by flow cytometry and antibodies used

| Marker (h = human) | Antibody |
|---|---|
| 7AAD (viability dye) | N/A |
| hCD45 | HI30 |
| mCD45 | 30-F11 |
| hCD33 | P67.6 |
| hCD3 | OKT3 |
| hCD11b | ICRF44 |
| hCD38 | HB-7 |
| hCD19 | HIB19 |
| hCD34 | 561 |
| hCD14 | MOP9 |
| hCD123 | 6H6 |
| hCD10 | HI10a |

Results from Cell Samples Obtained from the Blood of Engrafted Animals

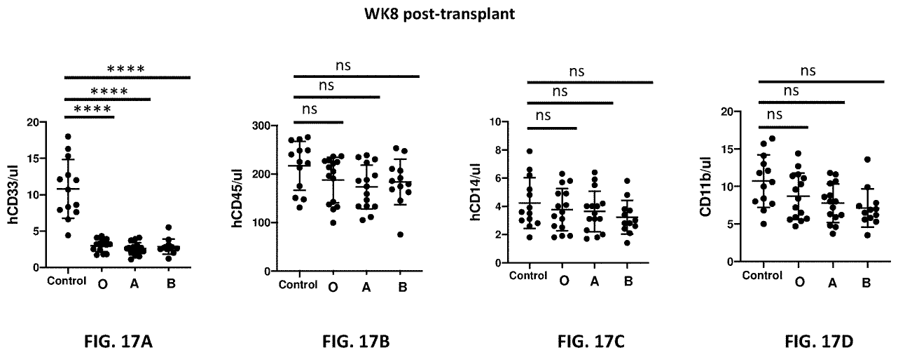

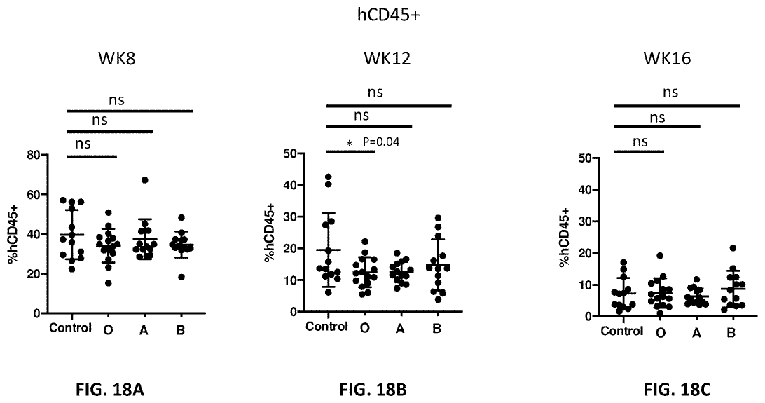

At week 8 post engraftment, the total numbers of cells per μL expressing hCD33 (FIG. 17A), hCD45 (FIG. 17B), hCD14 (FIG. 17C) and hCD11b (FIG. 18D) were quantified in the mice that received CD33KO mPB CD34+ HSPCs cells edited with either gRNA A (A), gRNA B (B), or gRNA O (O), or mice that received the control gRNA edited cells (control, gCtrl). As shown in FIG. 17A, the mice that received the CD33KO cells (edited with gRNA: O, A, or B, as depicted on the X-axis), had very few hCD33+ cells (≥5 cells per μL) compared to the control cells. The numbers of hCD45+ cells, hCD14+, and CD11b+ cells were comparable across all mice regardless of which edited cells they were engrafted with. These results indicated successful engraftment of CD33KO cells edited with gRNA A or gRNA B in the blood of mice.

At weeks 8, 12, and 16 following engraftment, the percentage of nucleated blood cells that were hCD45+ was quantified in the four groups of mice (n=15 mice/group) that received control cells edited with the control gRNA (gCtrl), or the CD33KO cells (edited by gRNA: O, A, or B, as depicted on the X-axis). This was quantified by dividing the hCD45+ absolute cell count by the mouse CD45+(mCD45) absolute cell count (FIGS. 18A-18C). At weeks 8 (FIG. 18A), 12 (FIG. 18B), and 16 (FIG. 18D) post-engraftment, equivalent levels of hCD45+ cells were observed in the blood between control and CD33KO groups.

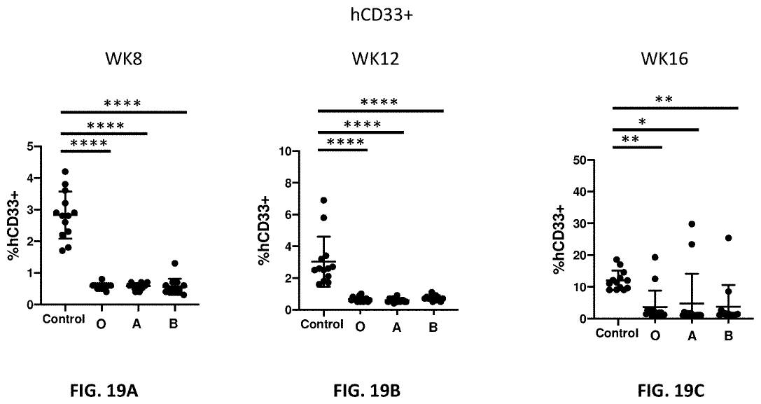

The percentage of hCD33+ cells in the blood was also quantified at week 8 (FIG. 19A), 12 (FIG. 19B), and 16 (FIG. 19C) following engraftment in the control and CD33KO mouse groups. As depicted in FIGS. 19A-19C, the mice engrafted with the CD33KO cells (edited with gRNA: O, A, or B, as depicted on the X-axis) had significantly lower levels of hCD33+ cells compared to the mice engrafted with control cells at weeks 8, 12 and 16. Further, engraftment of CD33KO cells edited with gRNA A or gRNA B resulted in similar, low levels of hCD33+ cells in the blood, as engraftment of CD33KO cells edited with the gRNA, gRNA O.

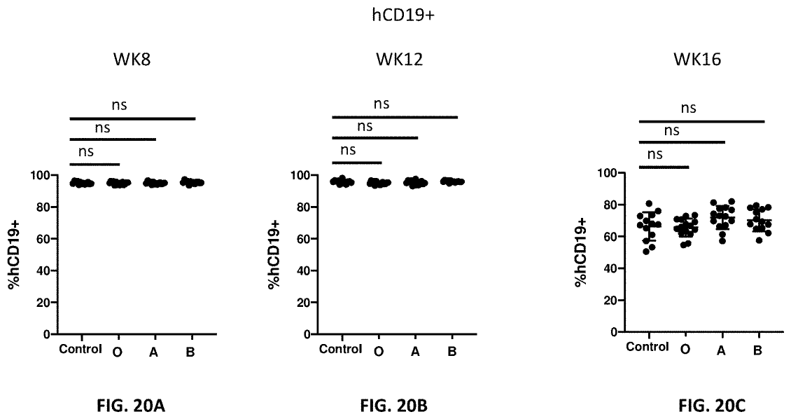

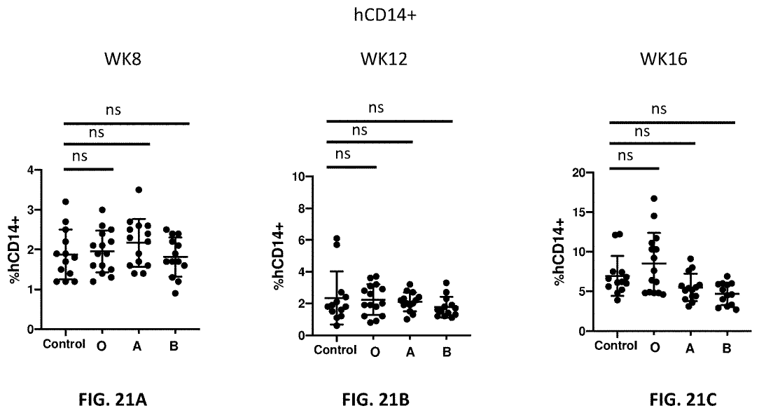

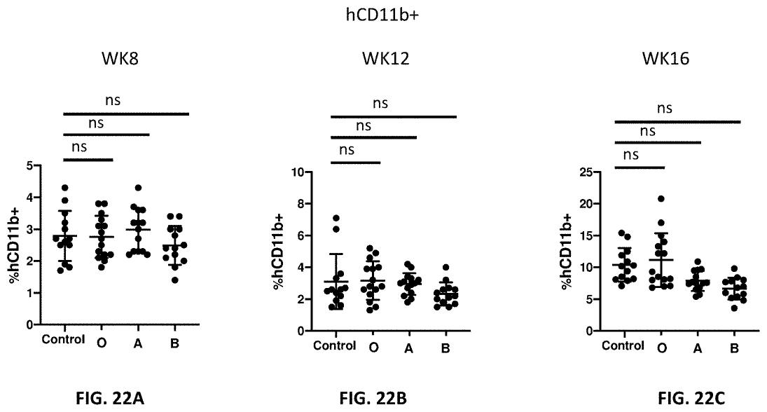

Next, the percentages of CD19+ lymphoid cells (FIGS. 20A-20C), hCD14+ monocytes (FIGS. 21A-21C), and hCD11b+ granulocytes/neutrophils (FIGS. 22A-22C) in the blood were quantified at week 8 (FIGS. 20A, 21A, 22A), week 12 (FIGS. 20B, 21B, 22B), and week 16 (FIGS. 20C, 21C, 22C) following engraftment in the mice engrafted with CD33KO cells (edited with gRNA: O, A, or B, as indicated on the X-axis) or control cells. The levels of hCD19+ cells, hCD14+ cells, and hCD11b+ cells in the blood were equivalent between the control and CD33KO groups, and the levels of these cells remained equivalent from weeks 8 to 16 post-engraftment. These data indicated that similar levels of human myeloid and lymphoid cell populations were present in mice that received the CD33KO cells and mice that received the control cells.

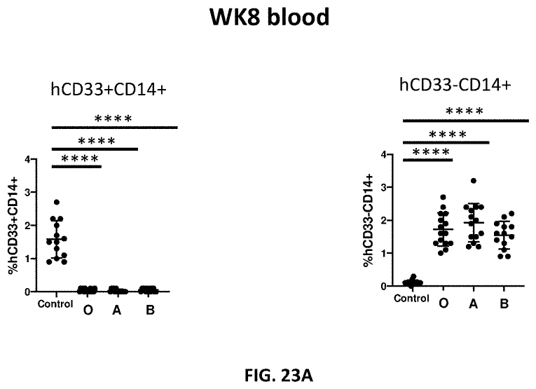

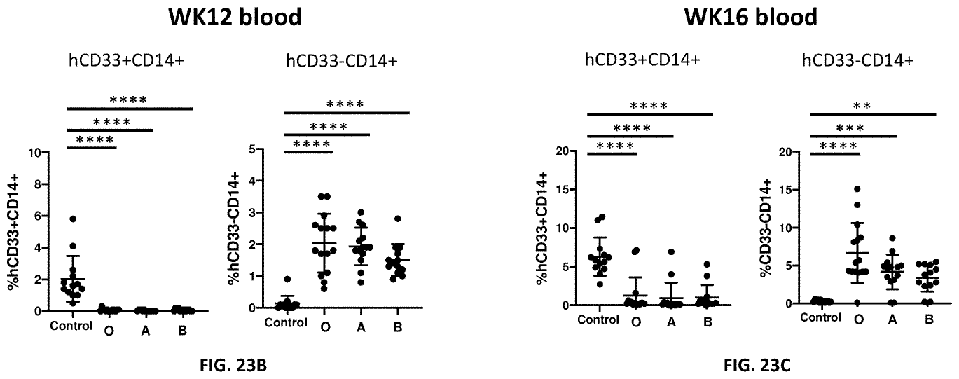

Finally, the percentage of human CD33KO derived monocyte cells (hCD33−CD14+) was quantified in the blood of mice engrafted with control cells and mice engrafted with CD33KO cells at weeks 8, 12, and 16 post engraftment (FIGS. 23A-23C, respectively). At week 8, in the mice engrafted with the control cells, hCD33+CD14+ monocytes were observed in the blood but no CD33KO derived monocytes (hCD33−CD14+) were observed (FIG. 23A, left graph). In contrast, in mice engrafted with the CD33KO cells (edited by gRNA: O, A or B, as depicted on the X-axis), no hCD33+CD14+ monocytes were observed, but approximately 1-3% of cells were CD33KO derived monocytes (hCD33−CD14+) (FIG. 23B, right graph). Similarly, at weeks 12 and 16 (FIGS. 23B and 23C, respectively), increasing percentages of CD33KO derived monocytes (hCD33−CD14+) were observed in mice engrafted with the CD33KO cells (FIGS. 23B and 23C, right graphs), whereas increasing numbers of hCD33+CD14 monocytes were observed in the control mice (FIGS. 23B and 23C, left graphs). These data indicate successful engraftment of CD33KO cells edited by gRNA A, gRNA B, or gRNA O which were able to expand and persist in the blood over time. Also, the total population of CD33KO derived monocytes (hCD33−CD14+) in the mice engrafted with the CD33KO cells was comparable to the hCD33+CD14+ monocytes in the mice engrafted with control cells at all time points analyzed.

Results from Cell Samples Obtained from the Bone Marrow of Engrafted Animals

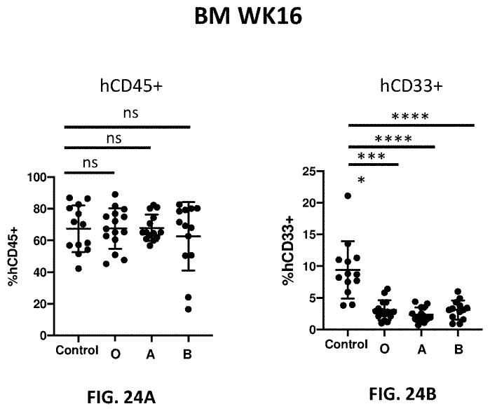

At week 16 post-engraftment, the percentages of hCD45+ cells (FIG. 24A) and the percentage of hCD33+ cells (FIG. 24B) were quantified in the bone marrow of mice that were engrafted with control cells or CD33KO cells (edited by gRNA: O, A, or B, as depicted on the X-axis). The percentage of hCD45+ cells was equivalent across control and CD33KO groups, indicating no loss of nucleated bone marrow cell frequency. The percentage of hCD33+ cells was significantly lower in the CD33KO groups compared to the control group, indicating loss of CD33 from nucleated blood cells in these groups. These data also demonstrate the long term persistence of CD33KO HSCs in the bone marrow of NSG mice.

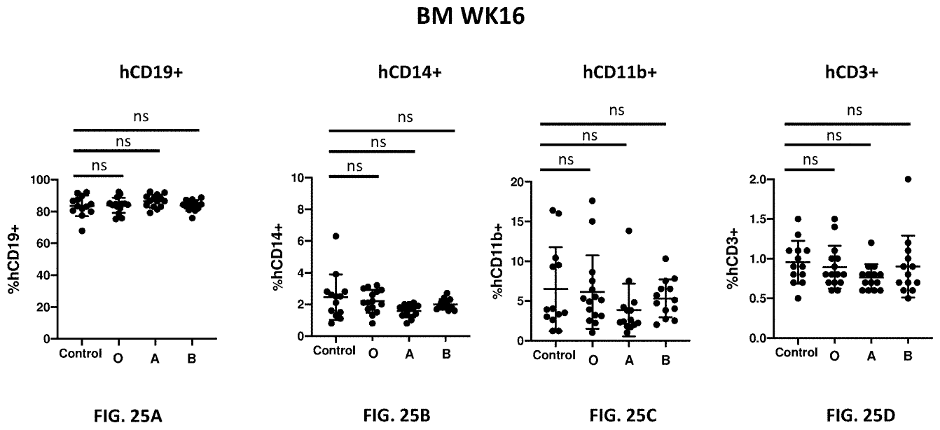

Additionally, at week 16 post engraftment, the percentages of CD19+ lymphoid cells (FIG. 25A), hCD14+ monocytes (FIG. 25B), hCD11b+ granulocytes/neutrophils (FIG. 25D), and hCD3+ T cells (FIG. 25E) in the bone marrow were quantified. The levels of hCD19+ cells, hCD14+ cells, hCD11b+ cells, and hCD3+ in the bone marrow were equivalent between the control and CD33KO groups. These data indicate multilineage human hematopoietic reconstitution from the edited CD33KO cells in the mice.

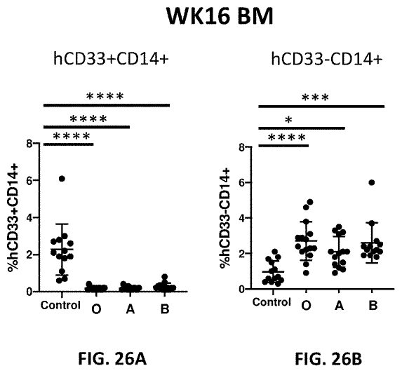

The percentages of CD33KO derived monocytes (hCD33−CD14+) (FIG. 26 B) and hCD33+CD14+ monocytes (FIG. 26A) were quantified in the control and CD33KO cell engrafted mice at week 16 post-engraftment. CD33KO derived monocytes (hCD33−CD14+) were observed in the bone marrow of mice engrafted with CD33KO cells (edited by gRNA: O, A or B, as depicted on the X-axis). Further, the CD33KO derived monocyte (hCD33-CD14+) population in the mice engrafted with the CD33KO cells remained comparable to the population of hCD33+CD14+ monocyte population observed in the mice engrafted with control cells at week 16 post-engraftment in the bone marrow of the NSG mice.

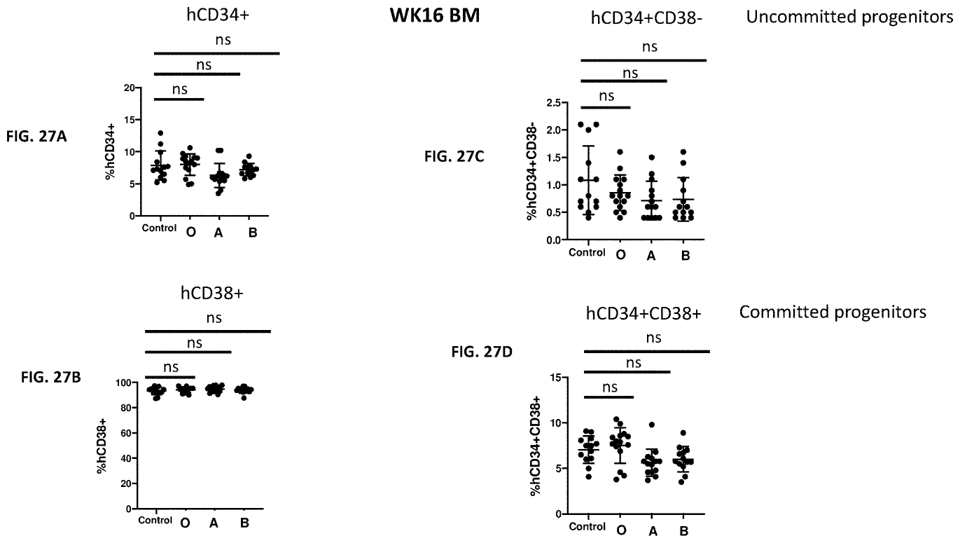

At week 16, the percentages of hCD34+ cells (FIG. 27A), hCD38+ cells (FIG. 27B), hCD34+hCD38− uncommitted progenitor cells (FIG. 27C), and hCD34+hCD38+ committed progenitor cells (FIG. 27D) were quantified in the bone marrow of mice engrafted with control cells or mice engrafted with CD33KO cells (edited by gRNA: O, A, or B, as depicted on the X-axis). These results demonstrated preserved progenitor cell population in the bone marrow of mice engrafted with CD33KO cells, as similar levels of hCD34+ cells, hCD38+ cells, hCD34+hCD38− uncommitted progenitor cells, and hCD34+hCD38+ committed progenitor cells were observed in the control and CD33KO groups.

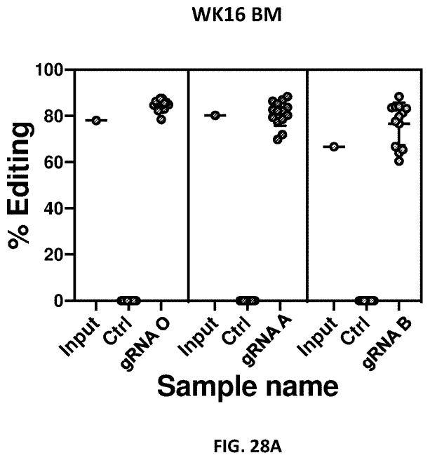

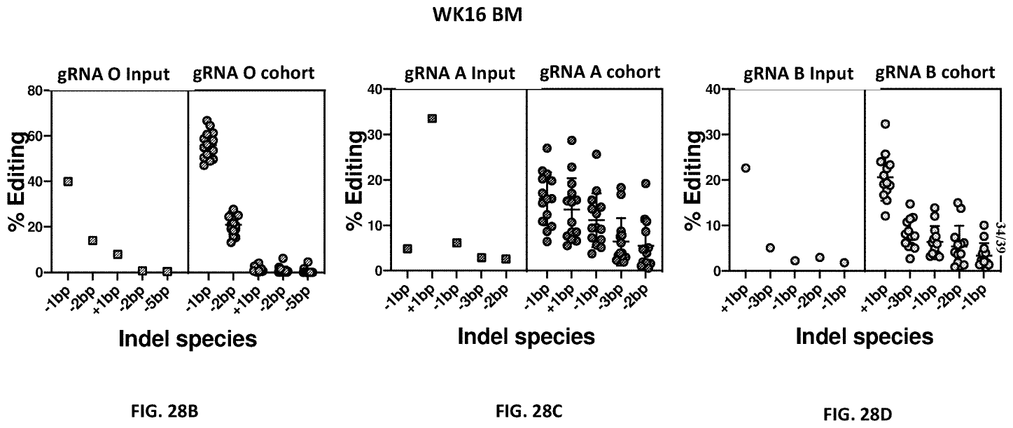

Finally, amplicon-seq was performed on bone marrow samples isolated at week 16 post-engraftment, to analyze the on-target CD33 editing in mice that were engrafted with the edited CD33KO cells. FIG. 28A demonstrates the percentage of edited cells in mice administered CD33KO cells that were edited with the following gRNAs: gRNA O (left panel), gRNA A (center panel), or gRNA B (right panel). All gRNAs used demonstrated a high percentage of on-targeted editing of CD33 (approximately 60-90%). FIGS. 28B-28D demonstrate the top 5 INDEL species representing different editing events observed in the isolated bone marrow cells, for each gRNA used in generating the CD33KO cells. gRNA A and gRNA B, comparable to gRNA O, resulted in a variety of insertions and deletions in the CD33 gene, ranging from 1 to 5 base pairs in size.

Results from Cell Samples Obtained from the Spleen of Engrafted Animals

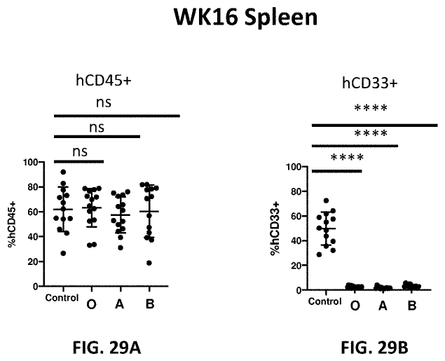

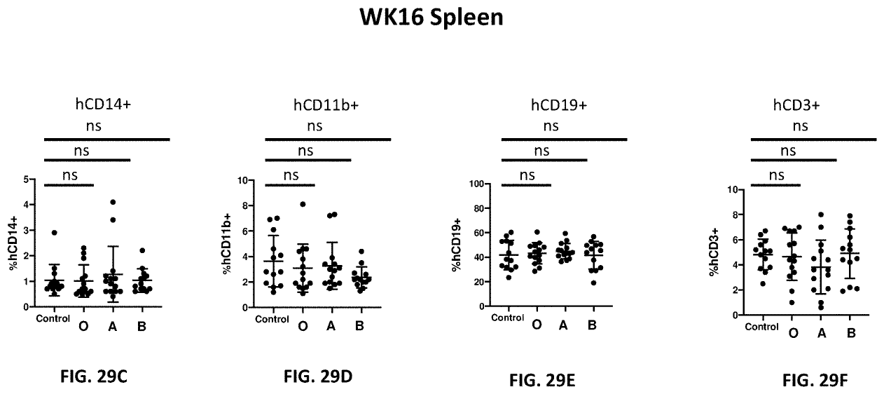

At week 16 post-engraftment, the percentages of hCD45+ cells (FIG. 29A) and the percentage of hCD33+ cells (FIG. 29B) were also quantified in the spleen of mice that were engrafted with control cells or CD33KO cells (edited by gRNA: O, A, or B, as depicted on the X-axis). The percentage of hCD45+ cells was equivalent across control and CD33KO groups. The percentage of hCD33+ cells was significantly lower in the CD33KO groups compared to the control group. These data also demonstrate the long term persistence of CD33KO HSCs in the spleens of NSG mice.

Additionally, at week 16 post engraftment, the percentages of hCD14+ monocytes (FIG. 29C), hCD11b+ granulocytes/neutrophils (FIG. 25D), CD19+ lymphoid cells (FIG. 29E), and hCD3+ T cells (FIG. 29F) in the spleen were quantified. The levels of hCD14+ cells, hCD11b+ cells, hCD19+ cells, and hCD3+ in the spleen were equivalent between the control and CD33KO groups. These data indicate that the edited CD33KO cells were capable of multilineage human hematopoietic cell reconstitution in the spleen of the NSG mice.

Results in the Blood and Bone Marrow Evaluating Neutrophils

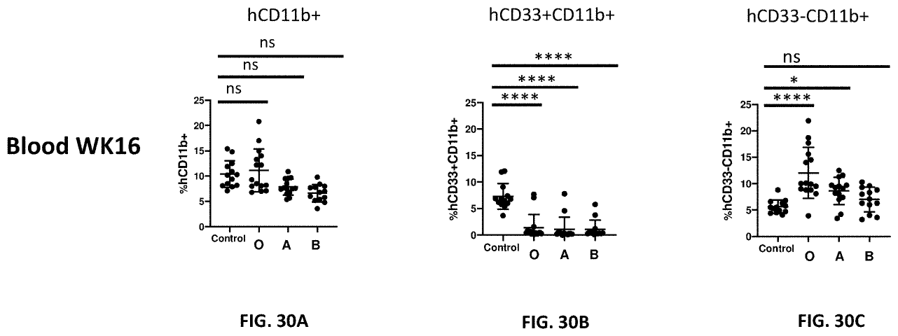

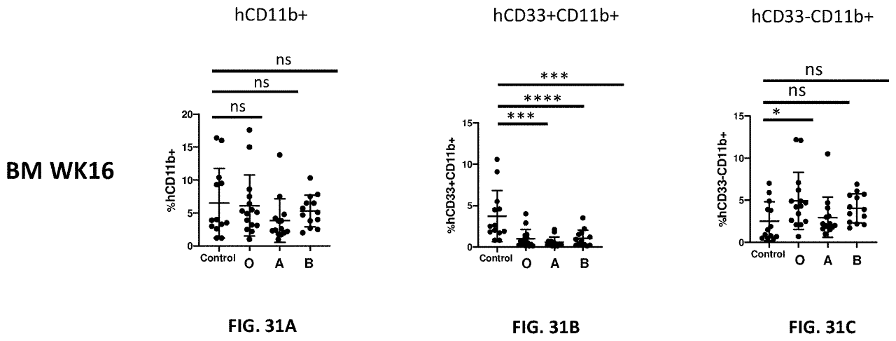

At week 16 post engraftment, the percentage of hCD11b+ cells (FIG. 30A (blood), 31A (bone marrow)), hCD33+CD11b+ neutrophil cells (FIG. 30B (blood), 31B (bone marrow)), and CD33KO derived neutrophils (hCD33-Cd11b+) (FIG. 30C (blood) 31C (bone marrow) were quantified in the blood and the bone marrow of mice engrafted with control cells or CD33KO cells (edited by gRNA: O, A, or B, as depicted on the X-axis). CD33KO derived neutrophils (hCD33−CD11b+) were observed in the blood and bone marrow of mice engrafted with CD33KO cells. Further, the CD33KO derived neutrophil (hCD33−CD11b+) population in the mice engrafted with the CD33KO cells remained comparable to the population of hCD33+CD11b+ neutrophil population observed in the mice engrafted with control cells at week 16 post-engraftment in both the blood and the bone marrow of the NSG mice.

Results in the Blood and Bone Marrow Evaluating Myeloid and Lymphoid Progenitor Cells

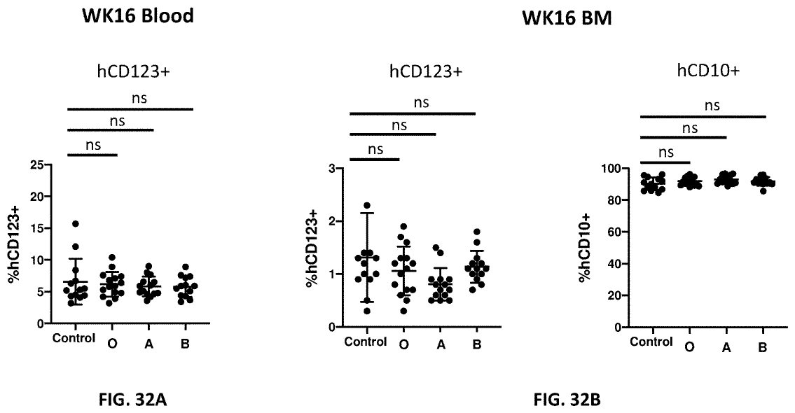

Also, at week 16, the percentage of hCD123+ cells in the blood (FIG. 32A) and the percentage of hCD123+ cells (FIG. 32B, left) in the bone marrow, and the percentage of hCD10+ cells (FIG. 32B, right) in the bone marrow were quantified in mice engrafted with control cells or CD33KO cells (edited by gRNA: O, A, or B, as depicted on the X-axis). These data show that the levels of myeloid and lymphoid progenitor cells were comparable at week 16 in the blood and the bone marrow for the control and CD33KO groups.

### OVERALL CONCLUSIONS

Taken together these data indicate that the CD33KO mPB CD34+ HSPCs edited by gRNA A or B resulted in successful engraftment and demonstrated long-term persistence in hematopoietic tissues, specifically the blood, bone marrow, and the spleen. Additionally, equivalent levels of human CD45+ cells and myeloid and lymphoid cell populations were observed in mice engrafted with control cells or the CD33KO mPB CD34+ HSPCs edited by gRNA A or B. Finally, amplicon-seq analysis demonstrated persistence at week 16 of on-target editing in all the mice engrafted with the CD33KO mPB CD34+ HSPCs edited by gRNA A or B.

### EQUIVALENTS AND SCOPE

Those skilled in the art will recognize, or be able to ascertain using no more than routine experimentation, many equivalents of the exemplary embodiments described herein. The scope of the present disclosure is not intended to be limited to the above description.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the invention encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, it is to be understood that every possible individual element or subgroup of the elements is also disclosed, and that any element or subgroup of elements can be removed from the group. It is also noted that the term “comprising” is intended to be open and permits the inclusion of additional elements, features, or steps. It should be understood that, in general, where an embodiment, is referred to as comprising particular elements, features, or steps, embodiments, that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims. For purposes of brevity, all of the embodiments in which one or more elements, features, purposes, or aspects is excluded are not set forth explicitly herein. The disclosure contemplates all combinations of any one or more of the foregoing embodiments, as well as combinations with any one or more of the embodiments set forth in the detailed description and examples.

Although methods and materials similar or equivalent to those described herein can be used in the practice or testing of the present invention, suitable methods and materials are described herein. All publications, patent applications, patents, and other references (e.g., sequence database reference numbers) mentioned herein are incorporated by reference in their entirety. For example, all GenBank, Unigene, and Entrez sequences referred to herein, e.g., in any Table herein, are incorporated by reference. Unless otherwise specified, the sequence accession numbers specified herein, including in any Table herein, refer to the database entries current as of May 23, 2019. When one gene or protein references a plurality of sequence accession numbers, all of the sequence variants are encompassed.

1. A gRNA comprising a targeting domain, wherein the targeting domain comprises a sequence of SEQ ID NO: 10.

2. A gRNA comprising a targeting domain, wherein the targeting domain comprises a sequence of SEQ ID NO: 9.

3. A gRNA comprising a targeting domain, wherein the targeting domain comprises a sequence of SEQ ID NO: 11.

4. A gRNA comprising a targeting domain, wherein the targeting domain comprises a sequence of SEQ ID NO: 12.

5. The gRNA of any of claim 1-4, which comprises a first complementarity domain, a linking domain, a second complementarity domain which is complementary to the first complementarity domain, and a proximal domain.

6. The gRNA of any of claims 1-5, which is a single guide RNA (sgRNA).

7. The gRNA of any of claims 1-6, which comprises one or more 2′O-methyl nucleotide.

8. The gRNA of any of claims 1-7, which comprises one or more phosphorothioate or thioPACE linkage.

9. A method of producing a genetically engineered cell, comprising:
(i) providing a hematopoietic stem or progenitor cell, and
(ii) introducing into the cell (a) a gRNA of any of claims 1-4d; and (b) a Cas9 molecule that binds the gRNA,
thereby producing the genetically engineered cell.

10. The method of claim 9, wherein the Cas molecule comprises a SpCas9 endonuclease, a SaCas9 endonuclease, or a Cpf1 endonuclease.

11. The method of claim 9 or 10, wherein (i) and (ii) are introduced into the cell as a pre-formed ribonucleoprotein complex.

12. The method of claim 11, wherein the ribonucleoprotein complex is introduced into the cell via electroporation.

13. A genetically engineered hematopoietic stem or progenitor cell, which is produced by a method of any of claims 9-12.

14. A cell population, comprising a plurality of the genetically engineered hematopoietic stem or progenitor cells of claim 13.

15. The cell population of claim 14, which further comprises one or more cells that comprise one or more non-engineered CD33 genes.

16. The cell population of claim 14 or 15, which expresses less than 20% of the CD33 expressed by a wild-type counterpart cell population.

17. The cell population of any of claims 14-16, which comprises both of hematopoietic stem cells and hematopoietic progenitor cells.

18. The cell population of any of claims 14-17, which further comprises a second mutation at a gene encoding a lineage-specific cell surface antigen other than CD33.

19. The cell population of claim 18, wherein the gene encoding a lineage-specific cell surface antigen other than CD33 is CLL-1 or CD123.

20. A method, comprising administering to a subject in need thereof a cell population of any of claims 14-19.

21. The method of claim 20, wherein the subject has a hematopoietic malignancy.

22. The method of claim 20 or 21, which further comprises administering to the subject an effective amount of an agent that targets CD33, wherein the agent comprises an antigen-binding fragment that binds CD33.


SEQUENCE LISTING (64 sequences)

SEQ ID NO: 1 - DNA, 20 bp, Artificial Sequence
  source: /note=“Description of Artificial Sequence Synthetic oligonucleotide”
ccccaggactactcactcct

SEQ ID NO: 2 - DNA, 20 bp, Artificial Sequence
  source: /note=“Description of Artificial Sequence Synthetic oligonucleotide”
accgaggagtgagtagtcct

SEQ ID NO: 3 - DNA, 20 bp, Artificial Sequence
  source: /note=“Description of Artificial Sequence Synthetic oligonucleotide”
ggtgggggcagctgacaacc

SEQ ID NO: 4 - DNA, 20 bp, Artificial Sequence
  source: /note=“Description of Artificial Sequence Synthetic oligonucleotide”
cggtgctcataatcacccca

SEQ ID NO: 5 - DNA, 20 bp, Artificial Sequence
  source: /note=“Description of Artificial Sequence Synthetic oligonucleotide”
aggagtgagtagtcctgggg

SEQ ID NO: 6 - DNA, 20 bp, Artificial Sequence
  source: /note=“Description of Artificial Sequence Synthetic oligonucleotide”
aggactactcactcctcggt

SEQ ID NO: 7 - DNA, 20 bp, Artificial Sequence
  source: /note=“Description of Artificial Sequence Synthetic oligonucleotide”
ggttgtcagctgcccccacc

SEQ ID NO: 8 - DNA, 20 bp, Artificial Sequence
  source: /note=“Description of Artificial Sequence Synthetic oligonucleotide”
tggggtgattatgagcaccg

SEQ ID NO: 9 - RNA, 20 nt, Artificial Sequence
  source: /note=“Description of Artificial Sequence Synthetic oligonucleotide”
ccccaggacuacucacuccu

SEQ ID NO: 10 - RNA, 20 nt, Artificial Sequence
  source: /note=“Description of Artificial Sequence Synthetic oligonucleotide”
accgaggagugaguaguccu

SEQ ID NO: 11 - RNA, 20 nt, Artificial Sequence
  source: /note=“Description of Artificial Sequence Synthetic oligonucleotide”
ggugggggcagcugacaacc

SEQ ID NO: 12 - RNA, 20 nt, Artificial Sequence
  source: /note=“Description of Artificial Sequence Synthetic oligonucleotide”
cggugcucauaaucacccca

SEQ ID NO: 13 - DNA, 1095 bp, Homo sapiens
atgccgctgctgctactgctgcccctgctgtgggcaggggccctggctatggatccaaatttctggctgcaagtgcaggagtcagtgacggtacaggagggtttgtgcgtcctcgtgccctgcactttcttccatcccataccctactacgacaagaactccccagttcatggttactggttccgggaaggagccattatatccagggactctccagtggccacaaacaagctagatcaagaagtacaggaggagactcagggcagattccgcctccttggggatcccagtaggaacaactgctccctgagcatcgtagacgccaggaggagggataatggttcatacttctttcggatggagagaggaagtaccaaatacagttacaaatctccccagctctctgtgcatgtgacagacttgacccacaggcccaaaatcctcatccctggcactctagaacccggccactccaaaaacctgacctgctctgtgtcctgggcctgtgagcagggaacacccccgatcttctcctggttgtcagctgcccccacctccctgggccccaggactactcactcctcggtgctcataatcaccccacggccccaggaccacggcaccaacctgacctgtcaggtgaagttcgctggagctggtgtgactacggagagaaccatccagctcaacgtcacctatgttccacagaacccaacaactggtatctttccaggagatggctcagggaaacaagagaccagagcaggagtggttcatggggccattggaggagctggtgttacagccctgctcgctctttgtctctgcctcatcttcttcatagtgaagacccacaggaggaaagcagccaggacagcagtgggcaggaatgacacccaccctaccacagggtcagcctccccgaaacaccagaagaagtccaagttacatggccccactgaaacctcaagctgttcaggtgccgcccctactgtggagatggatgaggagctgcattatgcttccctcaactttcatgggatgaatccttccaaggacacctccaccgaatactcagaggtcaggacccagtga

SEQ ID NO: 14 - DNA, 279 bp, Homo sapiens
acttgacccacaggcccaaaatcctcatccctggcactctagaacccggccactccaaaaacctgacctgctctgtgtcctgggcctgtgagcagggaacacccccgatcttctcctggttgtcagctgcccccacctccctgggccccaggactactcactcctcggtgctcataatcaccccacggccccaggaccacggcaccaacctgacctgtcaggtgaagttcgctggagctggtgtgactacggagagaaccatccagctcaacgtcacct

SEQ ID NO: 15 - protein, 118 aa, Artificial Sequence
  source: /note=“Description of Artificial Sequence Synthetic polypeptide”
QVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSS

SEQ ID NO: 16 - protein, 113 aa, Artificial Sequence
  source: /note=“Description of Artificial Sequence Synthetic polypeptide”
EIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIKR

SEQ ID NO: 17 - protein, 19 aa, Artificial Sequence
  source: /note=“Description of Artificial Sequence Synthetic peptide”
GSTSSGSGKPGSGEGSTKG

SEQ ID NO: 18 - protein, 107 aa, Artificial Sequence
  source: /note=“Description of Artificial Sequence Synthetic polypeptide”
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 19 - protein, 89 aa, Artificial Sequence
  source: /note=“Description of Artificial Sequence Synthetic polypeptide”
LSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 20 - protein, 38 aa, Artificial Sequence
  source: /note=“Description of Artificial Sequence Synthetic polypeptide”
CWLTKKKYSSSVHDPNGEYMFMRAVNTAKKSRLTDVTL

SEQ ID NO: 21 - protein, 97 aa, Artificial Sequence
  source: /note=“Description of Artificial Sequence Synthetic polypeptide”
IEVMYPPPYLDNEKSNGTIIHVKGKHLCPSPLFPGPSKPFWVLVVVGGVLACYSLLVTVAFIIFWVRSKRSRLLHSDYMFMRAVNTAKKSRLTDVTL

SEQ ID NO: 22 - protein, 113 aa, Artificial Sequence
  source: /note=“Description of Artificial Sequence Synthetic polypeptide”
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 23 - DNA, 20 bp, Artificial Sequence
  source: /note=“Description of Artificial Sequence Synthetic oligonucleotide”
ggtggctattgtttgcagtg

SEQ ID NO: 24 - DNA, 20 bp, Artificial Sequence
  source: /note=“Description of Artificial Sequence Synthetic oligonucleotide”
tttcttgagctgcagctggg

SEQ ID NO: 25 - DNA, 20 bp, Artificial Sequence
  source: /note=“Description of Artificial Sequence Synthetic oligonucleotide”
agttcccacatcctggtgcg

SEQ ID NO: 26 - DNA, 23 bp, Artificial Sequence
  source: /note=“Description of Artificial Sequence Synthetic oligonucleotide”
acctgtcaggtgaagttcgctgg

SEQ ID NO: 27 - DNA, 23 bp, Artificial Sequence
  source: /note=“Description of Artificial Sequence Synthetic oligonucleotide”
tggccgggttctagagtgccagg

SEQ ID NO: 28 - DNA, 23 bp, Artificial Sequence
  source: /note=“Description of Artificial Sequence Synthetic oligonucleotide”
ggccgggttctagagtgccaggg

SEQ ID NO: 29 - DNA, 23 bp, Artificial Sequence
  source: /note=“Description of Artificial Sequence Synthetic oligonucleotide”
caccgaggagtgagtagtcctgg

SEQ ID NO: 30 - DNA, 23 bp, Artificial Sequence
  source: /note=“Description of Artificial Sequence Synthetic oligonucleotide”
tccagcgaacttcacctgacagg

SEQ ID NO: 31 - DNA, 20 bp, Artificial Sequence
  source: /note=“Description of Artificial Sequence Synthetic oligonucleotide”
gctgtggggagaggggttgt

SEQ ID NO: 32 - DNA, 20 bp, Artificial Sequence
  source: /note=“Description of Artificial Sequence Synthetic oligonucleotide”
ctgtggggagaggggttgtc

SEQ ID NO: 33 - DNA, 20 bp, Artificial Sequence
  source: /note=“Description of Artificial Sequence Synthetic oligonucleotide”
tggggaaacgagggtcagct

SEQ ID NO: 34 - DNA, 20 bp, Artificial Sequence
  source: /note=“Description of Artificial Sequence Synthetic oligonucleotide”
gggcccctgtggggaaacga

SEQ ID NO: 35 - DNA, 20 bp, Artificial Sequence
  source: /note=“Description of Artificial Sequence Synthetic oligonucleotide”
agggcccctgtggggaaacg

SEQ ID NO: 36 - DNA, 20 bp, Artificial Sequence
  source: /note=“Description of Artificial Sequence Synthetic oligonucleotide”
gctgaccctcgtttccccac

SEQ ID NO: 37 - DNA, 20 bp, Artificial Sequence
  source: /note=“Description of Artificial Sequence Synthetic oligonucleotide”
ctgaccctcgtttccccaca

SEQ ID NO: 38 - DNA, 20 bp, Artificial Sequence
  source: /note=“Description of Artificial Sequence Synthetic oligonucleotide”
tgaccctcgtttccccacag

SEQ ID NO: 39 - DNA, 20 bp, Artificial Sequence
  source: /note=“Description of Artificial Sequence Synthetic oligonucleotide”
ccatagccagggcccctgtg

SEQ ID NO: 40 - DNA, 20 bp, Artificial Sequence
  source: /note=“Description of Artificial Sequence Synthetic oligonucleotide”
gcatgtgacaggtgaggcac

SEQ ID NO: 41 - DNA, 20 bp, Artificial Sequence
  source: /note=“Description of Artificial Sequence Synthetic oligonucleotide”
tgaggcacaggcttcagaag

SEQ ID NO: 42 - DNA, 20 bp, Artificial Sequence
  source: /note=“Description of Artificial Sequence Synthetic oligonucleotide”
aggcttcagaagtggccgca

SEQ ID NO: 43 - DNA, 20 bp, Artificial Sequence
  source: /note=“Description of Artificial Sequence Synthetic oligonucleotide”
ggcttcagaagtggccgcaa

SEQ ID NO: 44 - DNA, 20 bp, Artificial Sequence
  source: /note=“Description of Artificial Sequence Synthetic oligonucleotide”
gtacccatgaacttcccttg

SEQ ID NO: 45 - DNA, 20 bp, Artificial Sequence
  source: /note=“Description of Artificial Sequence Synthetic oligonucleotide”
gtggccgcaagggaagttca

SEQ ID NO: 46 - DNA, 20 bp, Artificial Sequence
  source: /note=“Description of Artificial Sequence Synthetic oligonucleotide”
tggccgcaagggaagttcat

SEQ ID NO: 47 - DNA, 20 bp, Artificial Sequence
  source: /note=“Description of Artificial Sequence Synthetic oligonucleotide”
ggaagttcatgggtactgca

SEQ ID NO: 48 - DNA, 20 bp, Artificial Sequence
  source: /note=“Description of Artificial Sequence Synthetic oligonucleotide”
ttcatgggtactgcagggca

SEQ ID NO: 49 - DNA, 20 bp, Artificial Sequence
  source: /note=“Description of Artificial Sequence Synthetic oligonucleotide”
ctaaacccctcccagtacca

SEQ ID NO: 50 - DNA, 20 bp, Artificial Sequence
  source: /note=“Description of Artificial Sequence Synthetic oligonucleotide”
cactcacctgcccacagcag

SEQ ID NO: 51 - DNA, 20 bp, Artificial Sequence
  source: /note=“Description of Artificial Sequence Synthetic oligonucleotide”
ccctgctgtgggcaggtgag

SEQ ID NO: 52 - DNA, 20 bp, Artificial Sequence
  source: /note=“Description of Artificial Sequence Synthetic oligonucleotide”
tgggcaggtgagtggctgtg

SEQ ID NO: 53 - DNA, 20 bp, Artificial Sequence
  source: /note=“Description of Artificial Sequence Synthetic oligonucleotide”
ggtgagtggctgtggggaga

SEQ ID NO: 54 - DNA, 20 bp, Artificial Sequence
  source: /note=“Description of Artificial Sequence Synthetic oligonucleotide”
gtgagtggctgtggggagag

SEQ ID NO: 55 - DNA, 20 bp, Artificial Sequence
  source: /note=“Description of Artificial Sequence Synthetic oligonucleotide”
atccatagccagggcccctg

SEQ ID NO: 56 - DNA, 20 bp, Artificial Sequence
  source: /note=“Description of Artificial Sequence Synthetic oligonucleotide”
tccatagccagggcccctgt

SEQ ID NO: 57 - DNA, 20 bp, Artificial Sequence
  source: /note=“Description of Artificial Sequence Synthetic oligonucleotide”
tcgtttccccacaggggccc

SEQ ID NO: 58 - DNA, 20 bp, Artificial Sequence
  source: /note=“Description of Artificial Sequence Synthetic oligonucleotide”
tggctatggatccaaatttc

SEQ ID NO: 59 - DNA, 23 bp, Artificial Sequence
  source: /note=“Description of Artificial Sequence Synthetic oligonucleotide”
agaaatttggatccatagccagg

SEQ ID NO: 60 - DNA, 23 bp, Artificial Sequence
  source: /note=“Description of Artificial Sequence Synthetic oligonucleotide”
atccctggcactctagaacccgg

SEQ ID NO: 61 - DNA, 23 bp, Artificial Sequence
  source: /note=“Description of Artificial Sequence Synthetic oligonucleotide”
cctcactagacttgacccacagg

SEQ ID NO: 62 - DNA, 20 bp, Artificial Sequence
  source: /note=“Description of Artificial Sequence Synthetic oligonucleotide”
cacagcgttatctccctctg

SEQ ID NO: 63 - protein, 12 aa, Artificial Sequence
  source: /note=“Description of Artificial Sequence Synthetic peptide”
GGGSGGGSGGGS

SEQ ID NO: 64 - DNA, 20 bp, Artificial Sequence
  source: /note=“Description of Artificial Sequence Synthetic oligonucleotide”
cctcactagacttgacccac